(19) United States
(12) Patent Application Publication
(10) Pub. No.: US 2011/0136893 A1
(43) Pub. Date: Jun. 9, 2011

(54) OLIGONUCLEOTIDE-, PROTEIN AND/OR PEPTIDE-POLYMER CONJUGATES

(76) Inventors: Karl-Hermann Schlingensiepen, Donaustauf (DE); Reimar Schlingensiepen, Regensburg (DE); Dagmar Fischer, Regensburg (DE); Andreas Mitsch, Lappersdorf-Piehlmuhle (DE)

(21) Appl. No.: 12/516,439

(22) PCT Filed: Dec. 21, 2007
(86) PCT No.: PCT/EP07/64494

§ 371 Date: Mar. 17, 2010

(30) Foreign Application Priority Data
Dec. 22, 2006 (EP) 06127 100.3

(51) Int. Cl. A61K 31/7088; C07H 21/00; C07H 21/02; A61P 35/00; A61P 31/12; A61P 35/02; A61P 35/04; A61P 31/20; A61P 31/14; A61P 31/22

(52) U.S. Cl. 514/44 R; 536/23.1; 536/24.5

(57) ABSTRACT

A conjugate or compound comprising polyethyleneglycol and an oligonucleotide, wherein at least one polyethyleneglycol is linked to the 5′-end of the oligonucleotide and at least one polyethyleneglycol is linked to the 3′-end of the oligonucleotide, wherein the molecular weight of the polyethyleneglycol linked to the 5′- and 3′-end of the oligonucleotide is identical and is <5000 Da, or wherein the molecular weight of the polyethyleneglycol linked to the 5′- and 3′-end of the oligonucleotide is different.

The present invention is directed to oligonucleotide-, protein- and/or peptide polymer conjugates.

Polymers have been widely used in biomaterial, biotechnology and medicine primarily because they are nonimmunogenic and water-soluble (Zhao 1997). In the area of drug delivery, polymer derivatives have been widely used in covalent attachment to proteins to reduce immunogenicity, proteolysis, kidney clearance and to enhance solubility. Similarly, polyethyleneglycol has been attached to low molecular weight, relatively hydrophobic drugs to enhance solubility, reduce toxicity and alter biodistribution.

Polyethyleneglycol is used as carrier or linked covalently to different therapeutics to enhance the cellular uptake. Another aim of linking polymers to therapeutics is to enlarge the molecular weight and increase the body halflife time. Reasons for this linkage are that those conjugates are described to show advantages such as reduced immunogenicity (Chirila 2001).

WO 2005/111238 A2 describes high molecular weight PEG-nucleic acid conjugates comprising a delivery aptamer and a therapeutic oligonucleotide sequence which is for example a CpG oligonucleotide, a siRNA oligonucleotide, an antisense oligonucleotide, or an aptamer. The conjugates described in this application have a molecular weight between 10 and 80 kDa and are intended to target the therapeutic oligonucleotide to a specific cell type.

Jäschke et al. (1994) disclose oligonucleotide conjugates comprising an oligonucleotide and polydisperse PEG linked to the 3′-end, 5′-end and/or internal positions of the oligonucleotide, wherein the oligonucleotide is synthesized by phosphoramidite chemistry. The conjugates contained a varying number of PEG-units, whereby the degree of polymerization of PEG ranged from 5 to 120 for 3′-terminal coupling and from 5 to 32 for 5′-terminal and internal coupling.

Moreover, WO 2007/109097 A1 describes amongst others iRNA, which includes 2′-modified ribose units and/or phosphorothioate linkages for example the 2′ hydroxyl group is modified by PEG. The molecular weight of PEG is not specified.

Aptamers conjugated with PEG at the 5′-end of the aptamer are suitable for the treatment, prevention, and/or amelioration of atopic diseases. The molecular weight of PEG is selected from the group consisting of 20, 30, 40, and 60 kDa, wherein PEG is linked to the 3′- and/or the 5′-end of an aptamer (cf. WO 2006/096222 A2).

Though there are a couple of advantages of PEG conjugates described in prior art, there is no teaching, that polymers could enhance the activity of therapeutics.

Surprisingly oligonucleotides, proteins and/or peptides such as immunostimulators linked with polymers of a certain weight, in particular linked with polymers of low molecular weight or a combination of low and high molecular weight polymers, show increased activity as well as pharmacological advantages despite of their higher molecular weight. One aspect of this invention is an oligonucleotide, a protein and/or a peptide, e.g., an immunostimulator linked with at least one polymer.

A further aspect of this invention is the synthesis of these compounds and their use for the preparation of a pharmaceutical composition as well as a method of the treatment of cancer, fibrosis, eye diseases such as glaucoma and/or viral infections such as HIV, FIV, HVA, HVB, HVC or influenza with this conjugate or compound.

The oligonucleotides, proteins and/or peptides, particularly an immunostimulator linked with at least one polymer (also referred to as “conjugate” or “compound” in the context of this invention) might have for example reduced toxicity especially in kidney and liver. This compound may show further advantages such as at least one of reduced lymphocytopenia, reduced leukocytopenia, reactivation of reduced clotting, reduction of thrombocytopenia, reduced immunogenicity, antigenicity, prolonged circulation halflife time, decreased kidney excretion, modified organ uptake, reduced uptake in organs of the reticuloendothelial system, decreased plasma protein binding, or increased drug stability compared to unconjugated oligonucleotides. Further advantages of the conjugate or compound are for example at least one of reduced coagulation, reduced complement activation, especially reduced C5 complement activation, recovering of the decreased CH₅₀ concentration, or higher cardiovascular and ZNS safety.

Further advances of the conjugate or compound are for example at least one of enhanced cellular uptake, enhanced affinity for nucleic acid target, altered intracellular localization, enhanced safety, enhanced efficacy, or increased stability in the presence of nucleases.

Derivatization of an oligonucleotide, a protein or a peptide with a polymer for example non-immunogenic polymer has the potential to alter the pharmacokinetic and pharmacodynamic properties of the oligonucleotide, protein, or peptide making it a more effective therapeutic agent. In particular polyethyleneglycol linked to an oligonucleotide, a protein and/or a peptide is suitable for example for altering solubility characteristics in aqueous or organic solvents, for modulation of the immune response, to increase the stability of an oligonucleotide, i.e., increases for example the resistance against exo- and endonucleases, a protein, or a peptide in solution, to enhance the halflife of substances in vivo and in vitro to aid in penetrating cell membranes, to alter pharmacological properties, to increase biocompatibility, or to prevent oligonucleotide, protein, or peptide adsorption to surfaces.

These beneficial properties of the modified oligonucleotides, proteins and/or peptides such as immunostimulator make them very useful in a variety of therapeutic applications.

Particularly advantageous effects such as increased halflife, decreased degradation, increased activity, etc. show oligonucleotides, proteins and/or peptides, which are linked to at least two polymers. Thereby the polymers are either identical or differ in the molecular weight and/or type of polymer. Preferably oligonucleotides, proteins and/or peptides combined with two or more polymers such as polyethyleneglycol (PEG) present a longer halflife, amongst others due to increased enzyme stability such as exonuclease, endonuclease, or proteinase stability, and an improved cell uptake. Depending on the size and structure of the polymer, a polymer with high or low molecular weight (MW) is more effective, or a combination of high and low molecular weight of a polymer such as polyalkylen oxide, e.g. PEG.

In particular polymers such as polyalkylen oxide, e.g., PEG increase the molecular size, often alter molecular charge, and ordinarily diminish receptor-binding capabilities leading to a reduction of the clearance rate. By sterically shielding the oligonucleotide-, protein-, or peptide-domain susceptible to enzymatic attack. PEG decreases the amount of oligonucleotide, protein, or peptide that is degraded and rendered biologically inactive. In parallel, by sterically masking the immunogenic/antigenic determinates of the therapeutic oligonucleotide, protein, or peptide, the polymer attachment leads to nonimmunogenic and nonantigenic conjugates.

### FIGURES

The Figures in the following are for better illustration of the invention; however, the invention is not limited to the illustrations presented in the Figures.

FIG. 1: The figure depicts the inhibition of the immunosuppressor TGF-beta2 in a cell culture incubated with different concentrations of the respective oligonucleotide which is an immunostimulator, respectively the oligonucleotide, here an immunostimulator, linked to one polymer at the 5′- or 3′-end, or two polymers, wherein one is linked to the 5′-end and one is linked to the 3′-end. The experiment was performed according to the description in example 13. The results shown refer to the oligonucleotide of SEQ ID NO 1 phosphorothioate (control) compared to phosphorothioate linked to one or two polyethyleneglycol. The vertical axis indicates the percentage of inhibition of suppression of the immunosuppressor TGF-beta2 (pg/Mio cells in % of untreated control). The black column (1) is the control (no inhibition), adjusted at 100%. The grey columns indicate the relative inhibition of TGF-beta2 by phosphorothioate of SEQ ID NO 1. The white columns indicate the inhibition of TGF-beta2 by the phosphorothioate of SEQ ID NO 1 linked with at least one polyethyleneglycol. Concentrations and weight of the linked polymers are shown in the table below. It can be clearly seen that the oligonucleotide linked to one polymer or linked to two polymers shows increased inhibition of TGF-beta2 compared to an oligonucleotide not linked to a polymer.

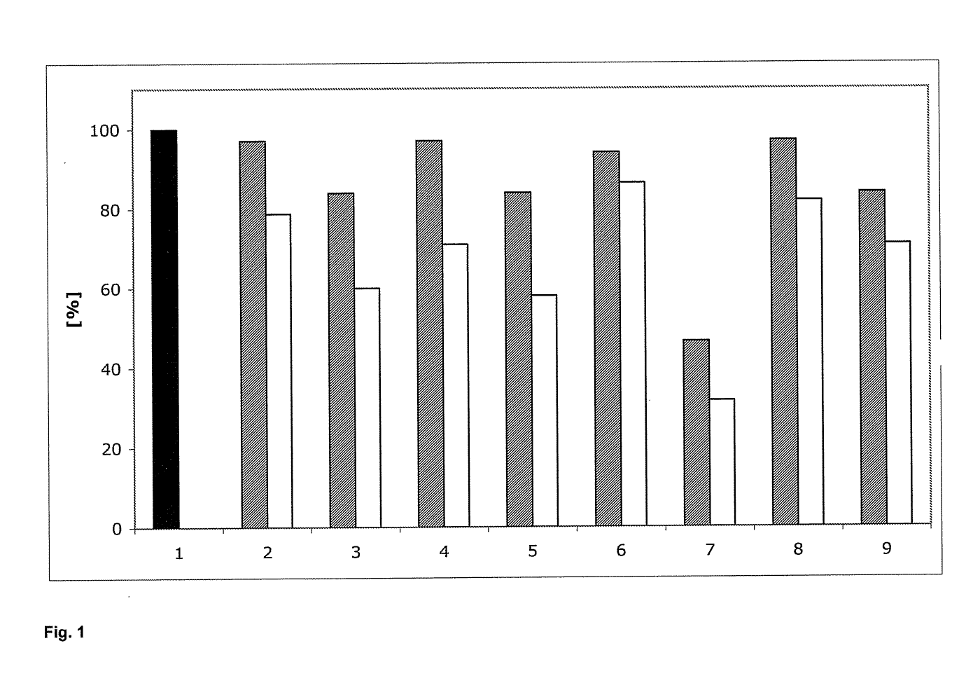

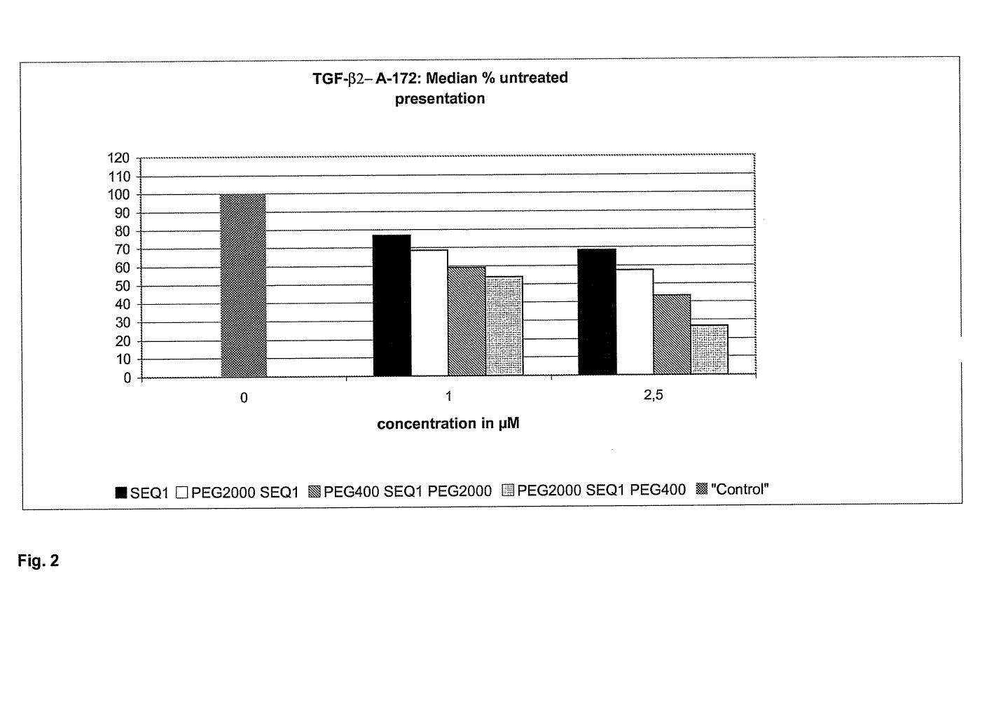

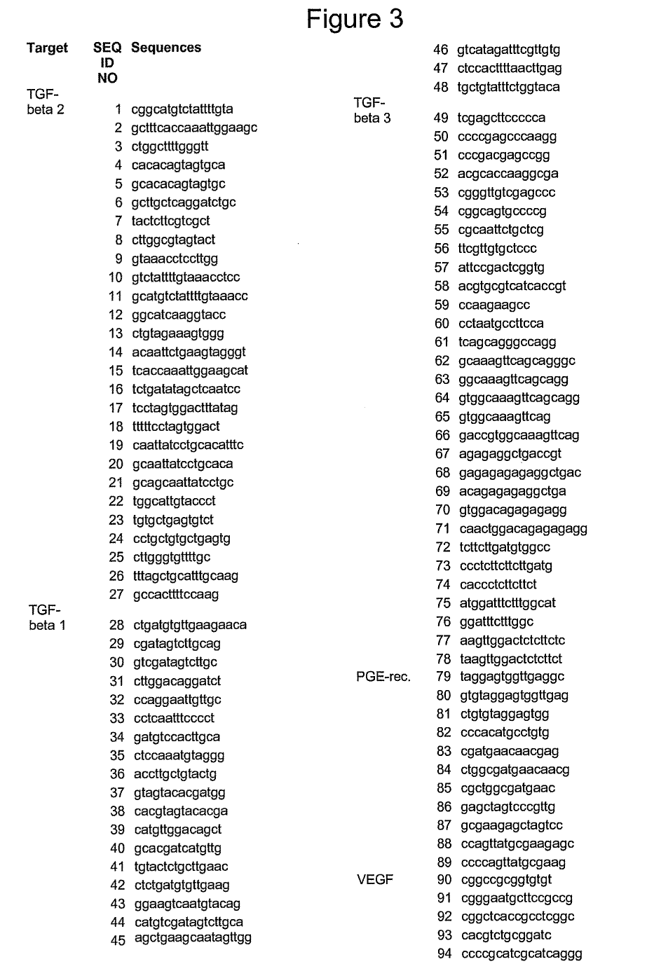

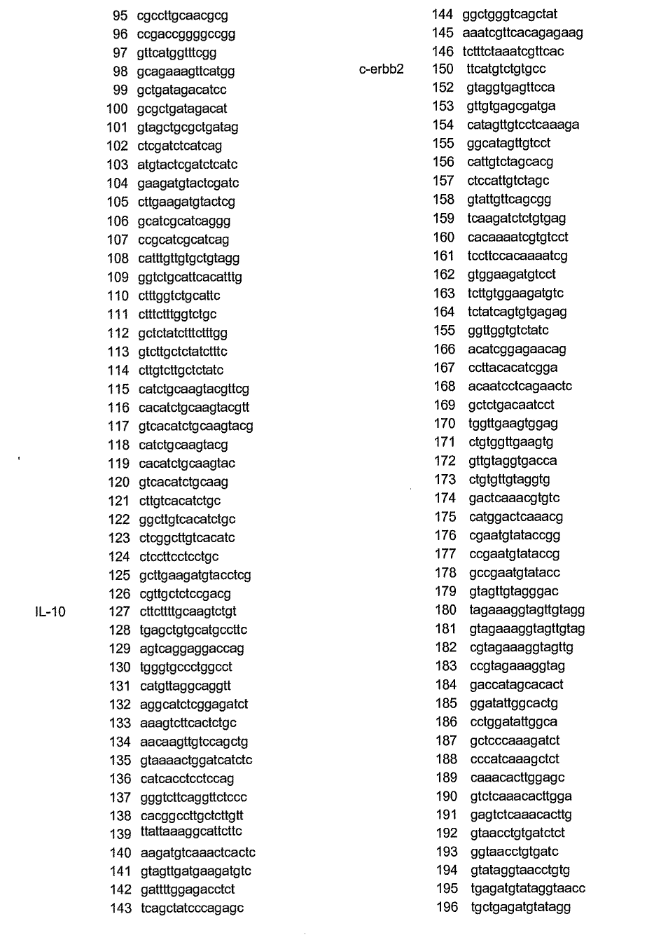

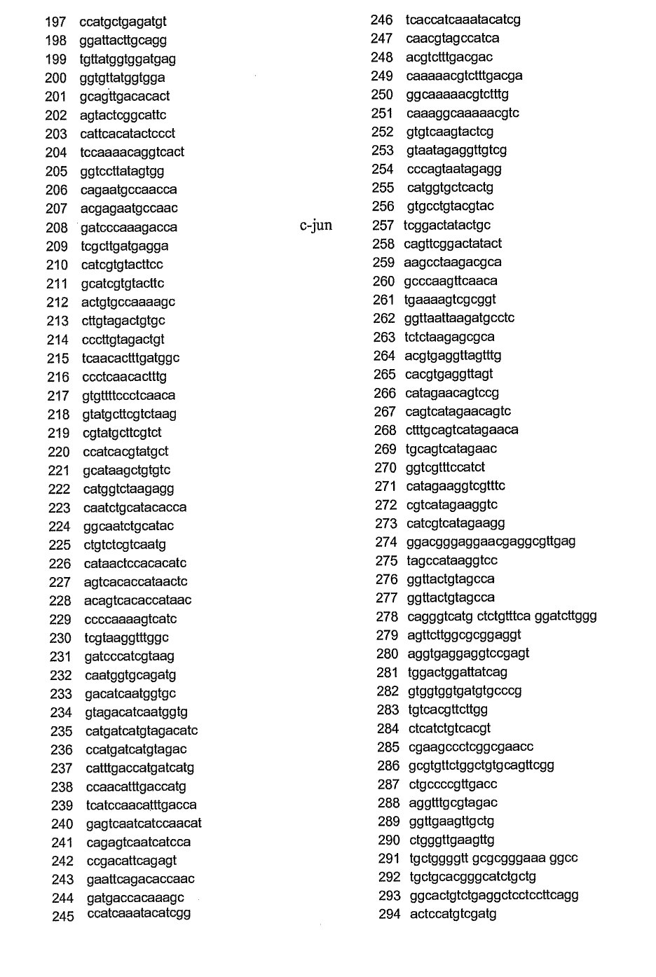

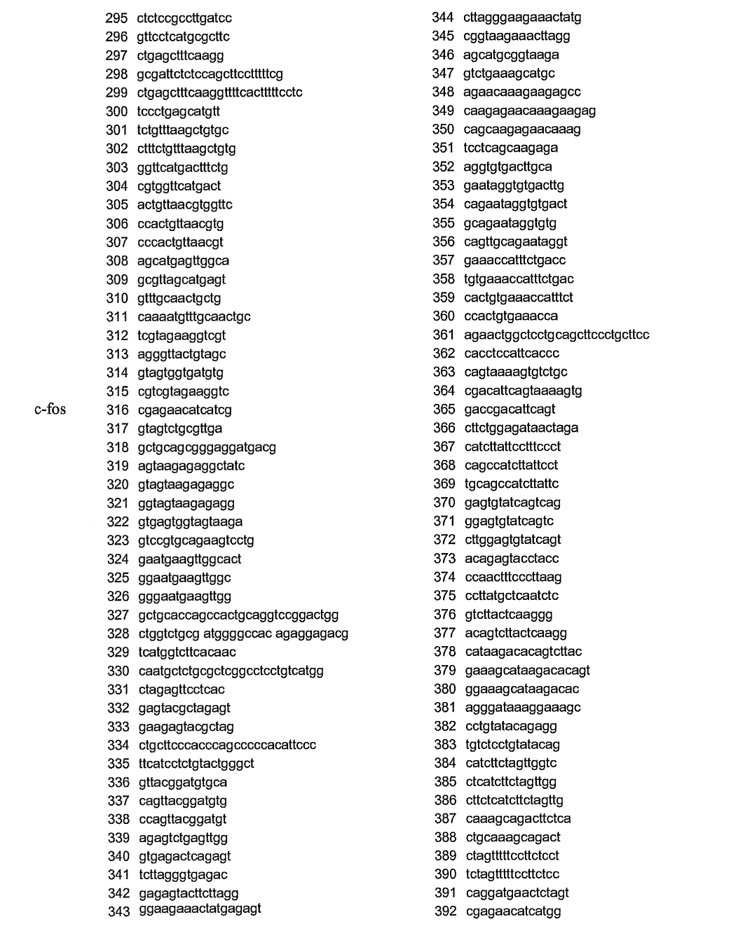

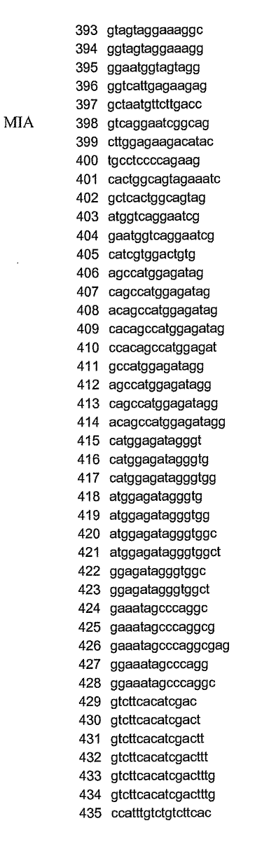

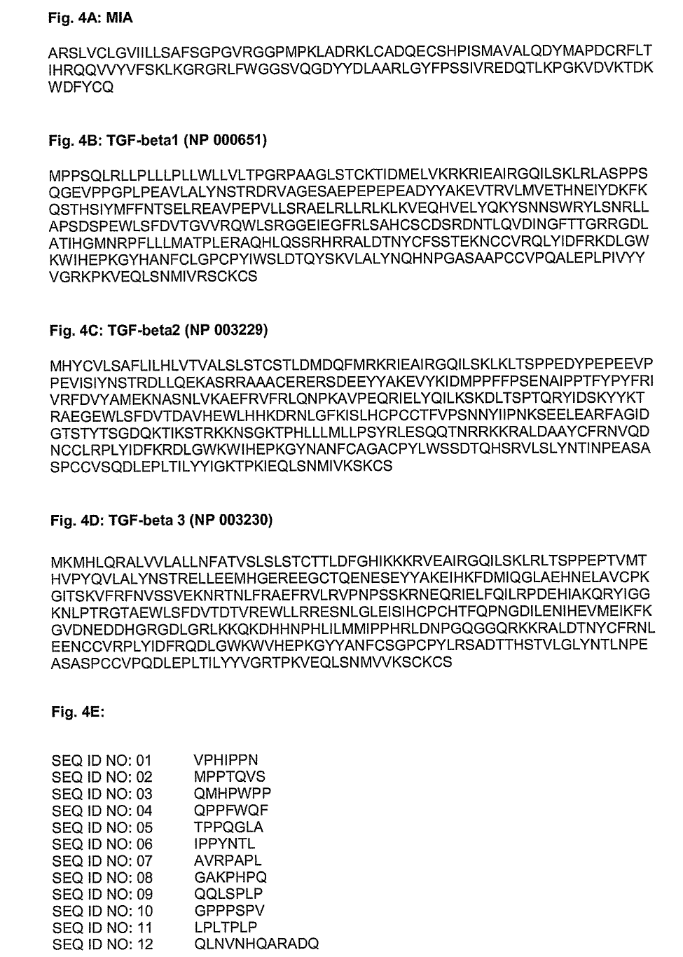

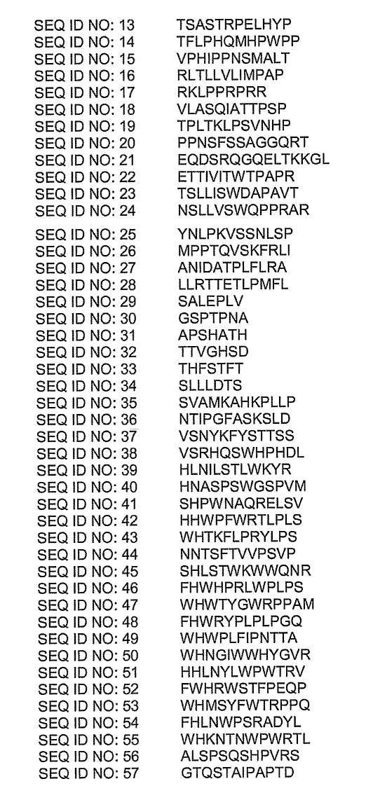

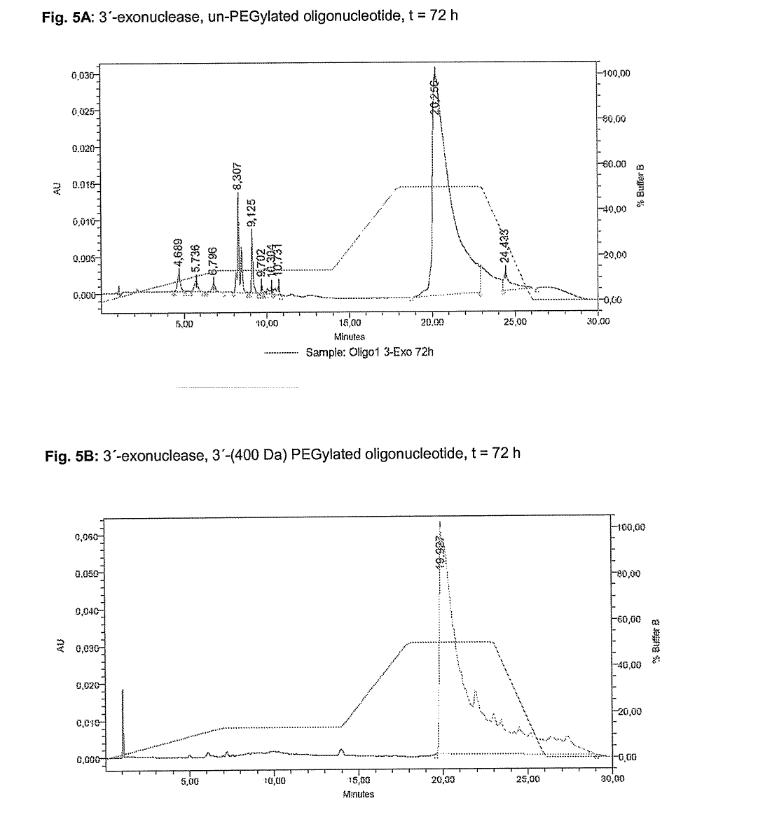

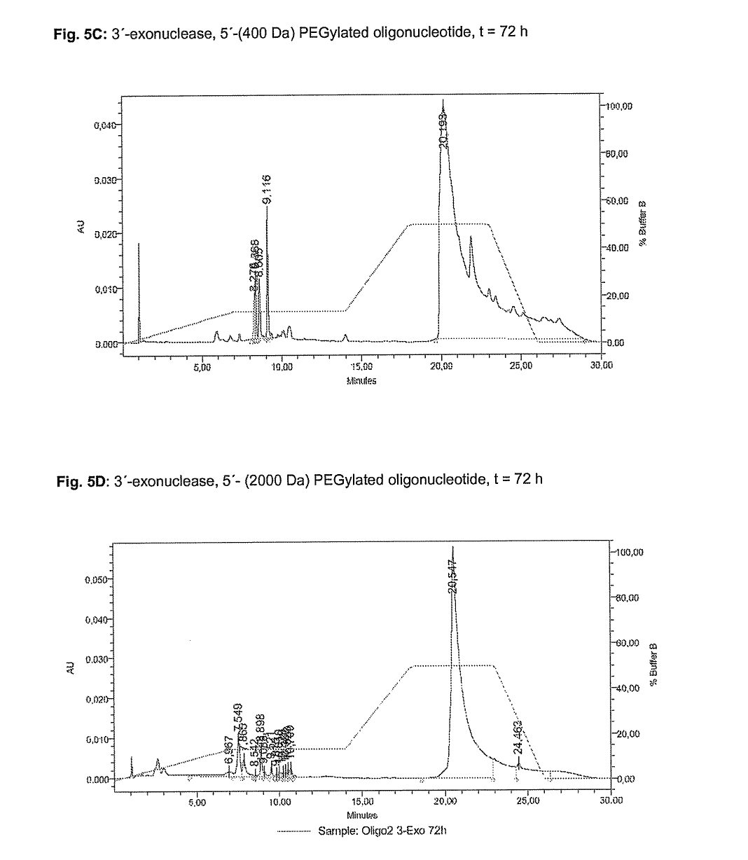

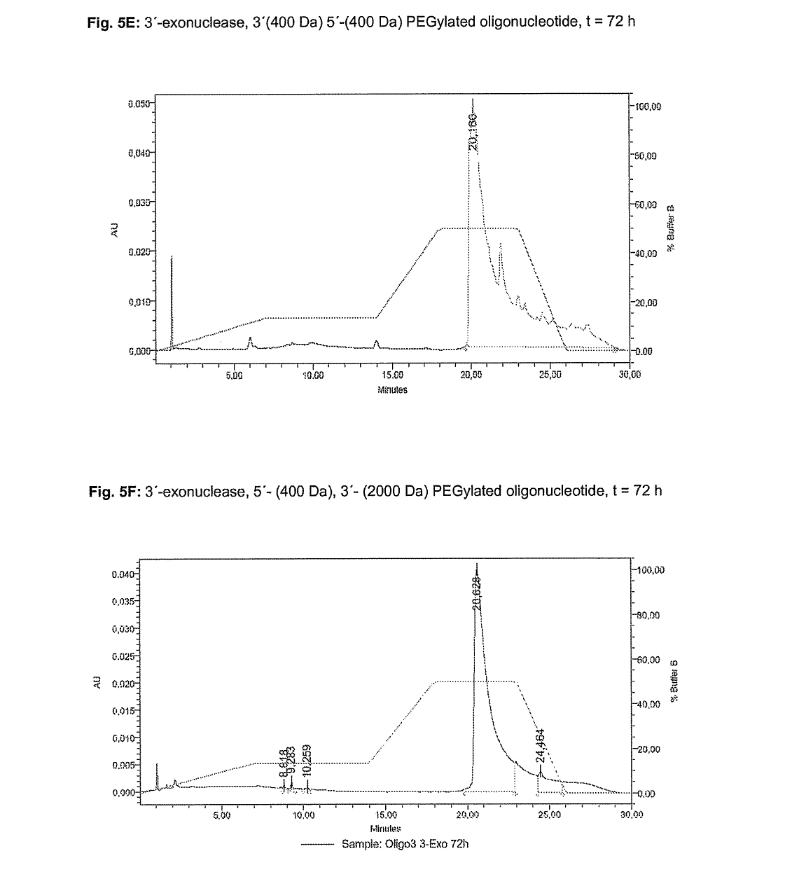

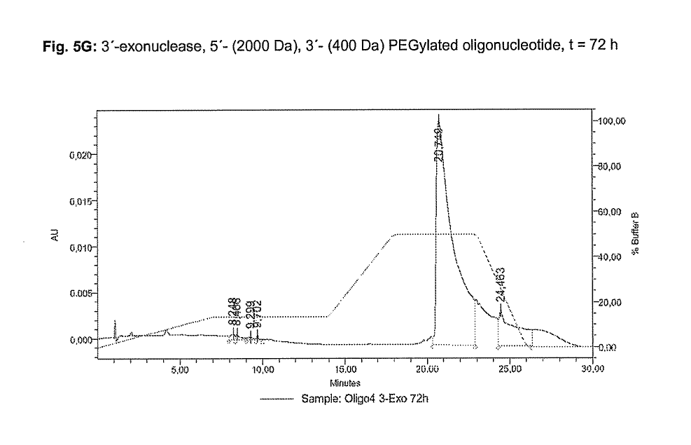

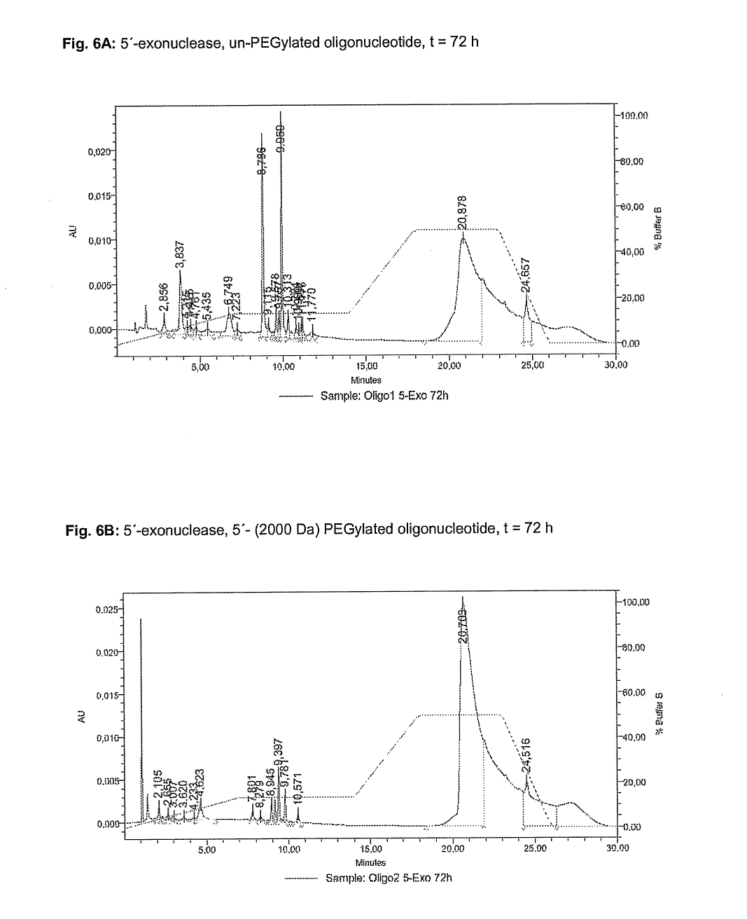

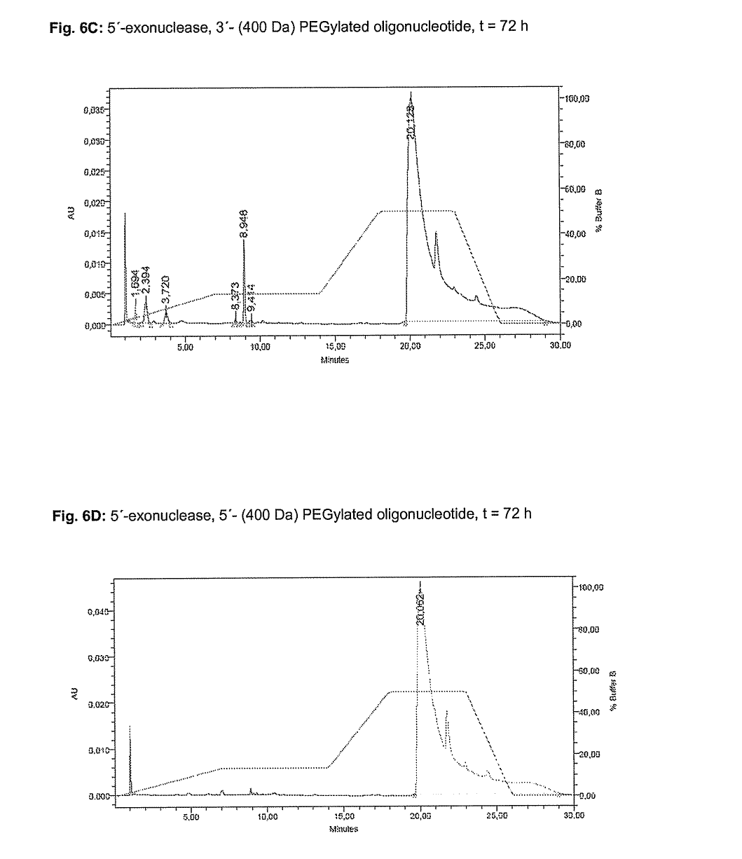

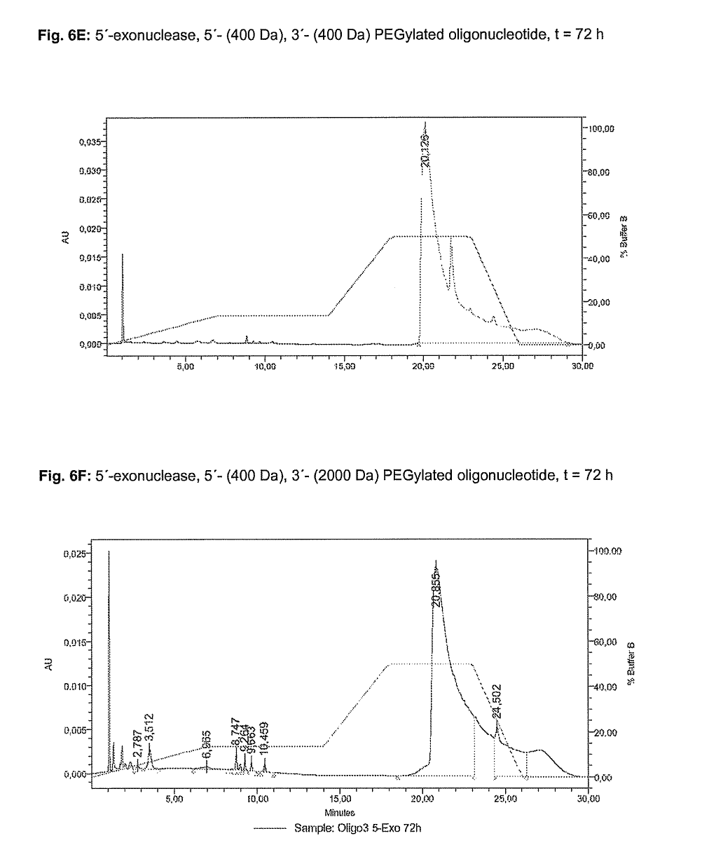

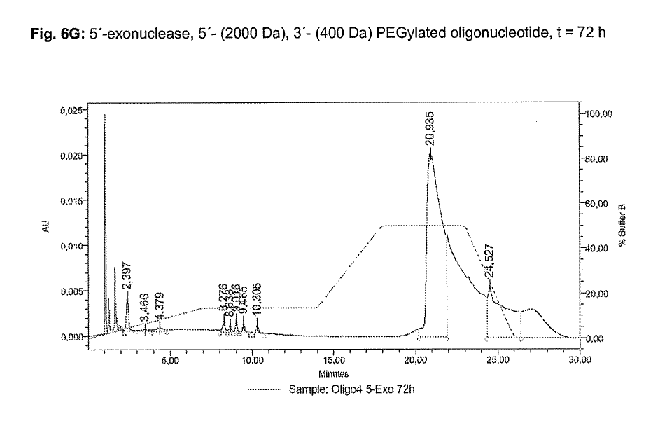

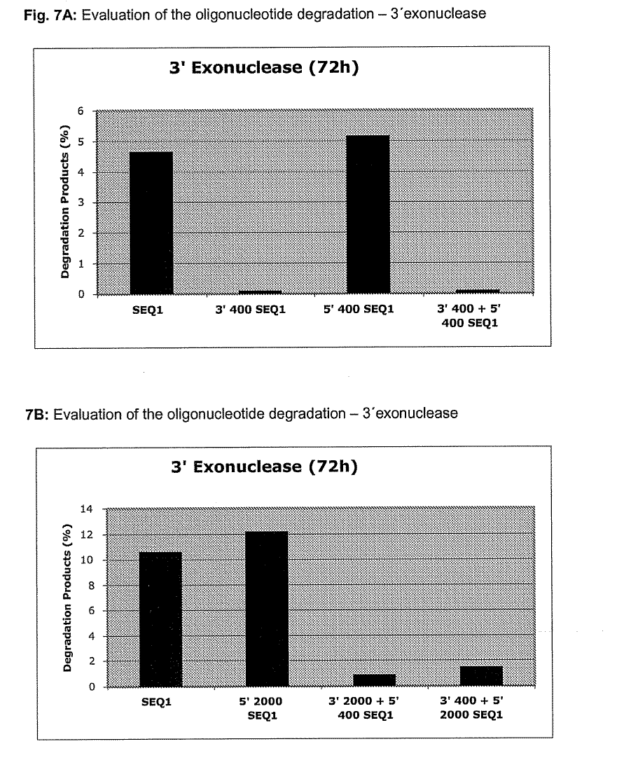

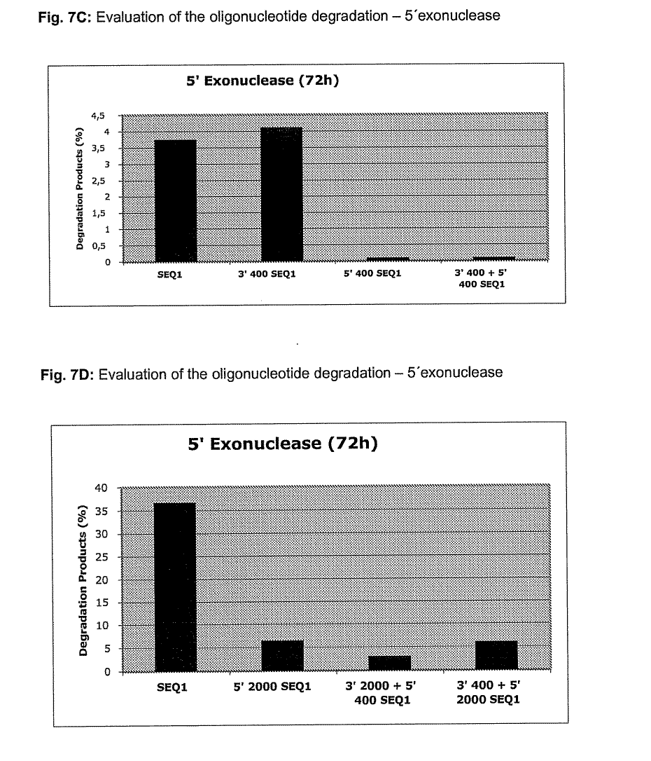

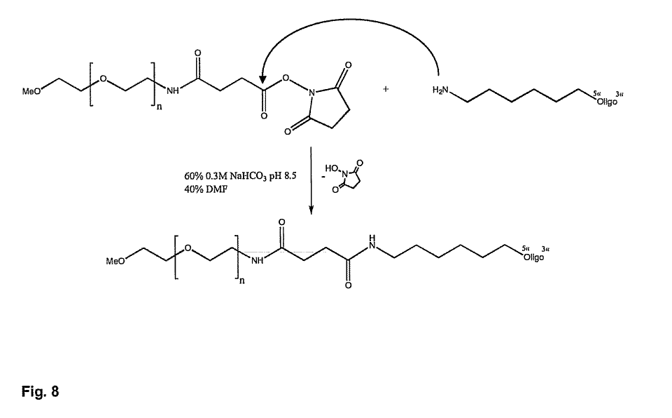

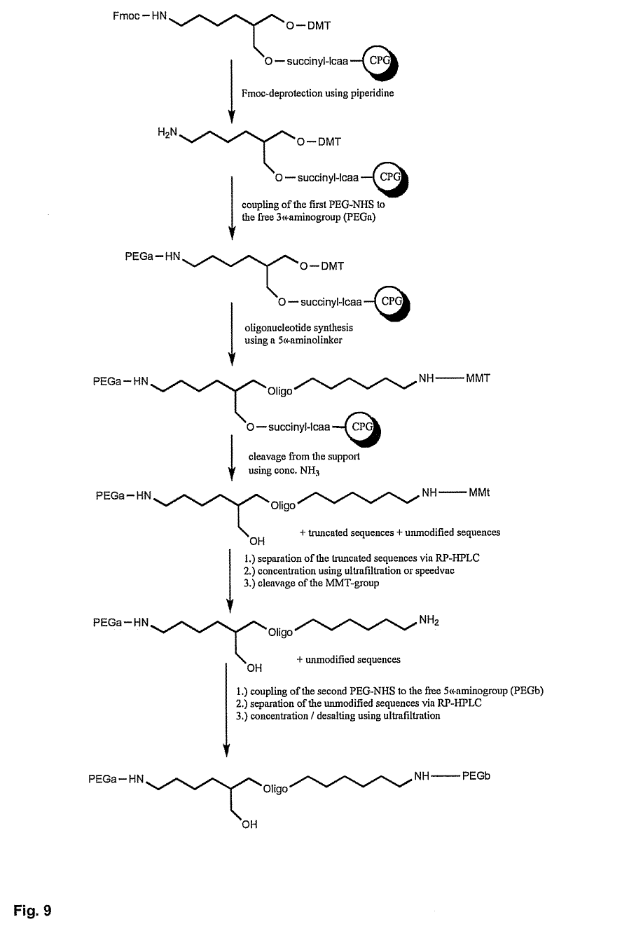

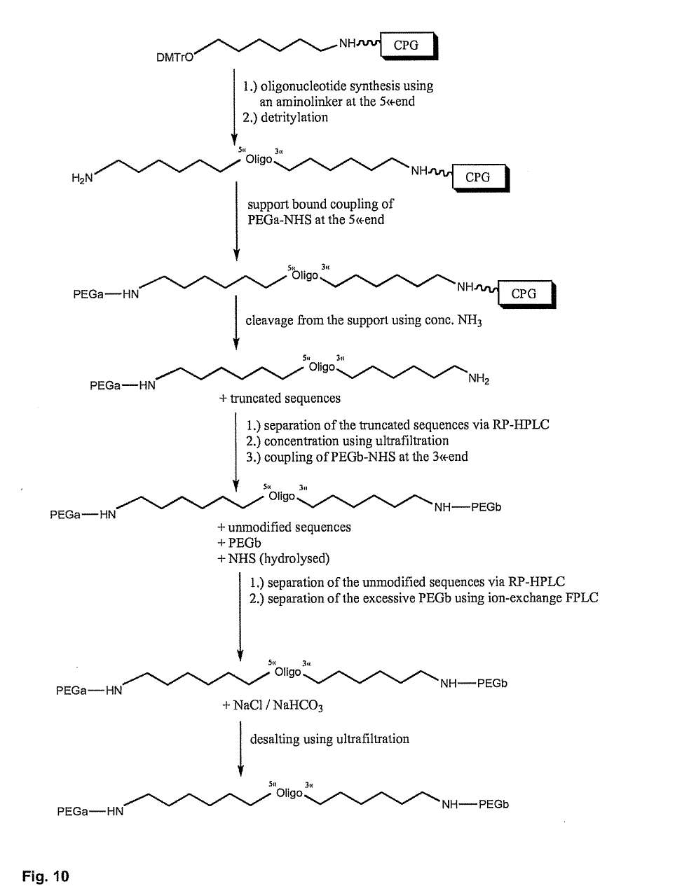

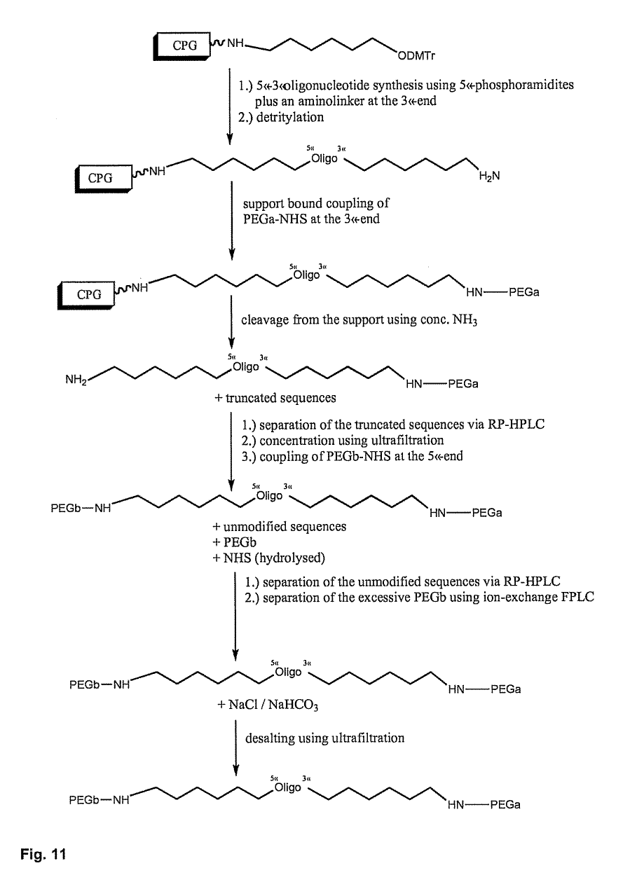

| | | Oligo- | | | |
|---|---|---|---|---|---|
| | Colour | nucleotide | | | TGF- |
| Col- | of the | (phosphoro- | Linked | Concen- | beta2 |
| umn | column | thioate) | polymer*) | tration | [%] |
| 1 | black | — | — | — | 100 |
| 2 | grey | SEQ ID NO1 | — | 0.2 microM | 97.1 |
| 2 | white | SEQ ID NO1 | PEG 400 | 0.2 microM | 78.8 |
| | | | (3′-end) | | |
| 3 | grey | SEQ ID NO1 | — | 1.0 microM | 84.0 |
| 3 | white | SEQ ID NO1 | PEG 400 | 1.0 microM | 60.0 |
| | | | (3′-end) | | |
| 4 | grey | SEQ ID NO1 | — | 0.2 microM | 97.0 |
| 4 | white | SEQ ID NO1 | 2 × PEG 400 | 0.2 microM | 71.0 |
| | | | (5′- and 3′-end) | | |
| 5 | grey | SEQ ID NO1 | — | 1.0 microM | 84.0 |
| 5 | white | SEQ ID NO1 | 2 × PEG 400 | 1.0 microM | 58.0 |
| | | | (5′- and 3′end) | | |
| 6 | grey | SEQ ID NO1 | — | 0.2 microM | 94.1 |
| 6 | white | SEQ ID NO1 | PEG 5000 | 0.2 microM | 86.5 |
| | | | (5′-end) | | |
| 7 | grey | SEQ ID NO1 | — | 40 microM | 46.5 |
| 7 | white | SEQ ID NO1 | PEG 5000 | 40 microM | 31.6 |
| | | | (5′-end) | | |
| 8 | grey | SEQ ID NO1 | — | 0.2 microM | 97.06 |
| 8 | white | SEQ ID NO1 | PEG 400 | 0.2 microM | 82.46 |
| | | | (5′ end) | | |
| 9 | grey | SEQ ID NO1 | — | 1.0 microM | 83.97 |
| 9 | white | SEQ ID NO1 | PEG 400 | 1.0 microM | 71.60 |
| | | | (5′ end) | | |

*)the average weight of the linked polymer is indicated in Da/mol

FIG. 2: The figure depicts the inhibition of the immunosuppressor TGF-beta2 in a cell culture incubated with different concentrations of the respective oligonucleotide, respectively the oligonucleotide linked with at least one polymer. The experiment was performed according to the description in example 13. The results are given from the oligonucleotide with SEQ ID NO 1 phosphorothioate (control) compared to SEQ ID NO 1 phosphorothioate (SEQ1) linked to one or two polyethyleneglycol (PEG) molecules. The vertical axis indicates the percentage of inhibition of suppression of the immunosuppressor TGF-beta2 (pg/Mio cells in % of untreated control). The horizontal axis indicates the concentration of the oligonucleotide in μM. In control samples no oligonucleotide was added (0 μM; control, checked column) to determine the TGF-beta2 baseline forming the reference value to calculate the % of inhibition indicated on the vertical axis. In the further samples oligonucleotides in the concentration of 1 μM, or 2.5 μM were added. The following oligonucleotides were tested: oligonucleotide of SEQ ID NO 1 without PEG (black column), oligonucleotide of SEQ ID NO 1 with PEG-2000 at the 5′-end of the oligonucleotide (white column), oligonucleotide of SEQ ID NO 1 with PEG-400 at the 5′-end and PEG-2000 at the 3′-end of the oligonucleotide (striped column), and oligonucleotide of SEQ ID NO 1 with PEG-2000 at the 5′-end and PEG-400 at the 3′-end of the oligonucleotide (dotted column). The data clearly indicate that an oligonucleotide linked to PEG has an increased inhibitory effect compared to an oligonucleotide without PEG, and a second PEG linked to the oligonucleotide increased the inhibitory effect of the oligonucleotide.

FIG. 3: This figure shows examples of oligonucleotide sequences of exemplaric genes TGF-beta1, TGF-beta2, TGF-beta3, PGE-rec., VEGF, IL-10, c-erbb2 (Her-2), c-jun, c-fos, and MIA.

FIG. 4: It presents examples of amino acid sequences of MIA (“Melanoma Inhibitory Activity”; FIG. 4A), TGF-beta1 (FIG. 4B), TGF-beta2 (FIG. 4C), and TGF-beta3 (FIG. 4D). Each amino acid sequence of 4A to 4D forming a peptide or protein fragment of the full length protein is, suitable to be linked with at least one polymer, forming polymer-peptide conjugates and compounds, respectively, or pharmaceutical compositions comprising or consisting of at least one of these conjugates or compounds in pharmaceutical acceptable carriers. FIG. 4E shows examples of peptides of MIA, fibronectin derived peptides and other peptides of the invention.

FIG. 5: The figures show 3′-exonuclease stability tests of an un-PEGylated oligonucleotide of SEQ ID NO 1 (FIG. 5A), of a 3′-PEGylated oligonucleotide (PEG-400, (FIG. 5B)), of a 5′-PEGylated oligonucleotide (PEG-400, (FIG. 5C), or PEG-2000, (FIG. 5D)), and of a 5′,3′-PEGylated oligonucleotide, wherein PEG-400 is at the 3′- and 5′-end (FIG. 5E), PEG-400 is at the 5′-end and PEG-2000 is at the 3′-end (FIG. 5F), or PEG-2000 is at the 5′-end and PEG-400 is at the 3′-end (FIG. 5G). The oligonucleotides were incubated with a 3′-exonuclease at 37° C. and were tested after 0, 1, 3, 6, 24, 48, 72, and 144 h. FIG. 5A to FIG. 5G show the degradation of the oligonucleotides after 72 h. The degradation increases with the time of incubation (increasing peak area (%) of impurities), wherein the oligonucleotides linked to PEG on the 3′-end, or on both ends are much more resistant to the 3′-exonuclease and the degradation is reduced.

FIG. 6: These figures show 5′-exonuclease stability tests of an un-PEGylated oligonucleotide of SEQ ID NO 1 (FIG. 6A), of a 5′-PEGylated oligonucleotide (PEG-2000, (FIG. 6B), and PEG-400, (FIG. 6D)), of a 3′-PEGylated oligonucleotide (PEG-400, (FIG. 6C)), and of a 5′,3′-PEGylated oligonucleotide, wherein PEG-400 is at the 3′- and 5′-end (FIG. 6E), PEG-400 is at the 5′-end and PEG-2000 is at the 3′-end (FIG. 6F), or PEG-2000 is at the 5′-end and PEG-400 is at the 3′-end (FIG. 6G). The oligonucleotides were incubated with a 5′-exonuclease at 37° C. and were tested after 0, 1, 3, 6, 24, 48, 72, and 144 h. FIG. 6A to FIG. 6G show the degradation of the oligonucleotide after 72 h. The degradation increases with the time of incubation (increasing peak area (%) of impurities), wherein the oligonucleotides linked to PEG on both ends are much more resistant to the 5′-exonuclease and thus, the degradation is reduced.

FIG. 7: These figures show an evaluation of the degradation of oligonucleotides with and without PEG by a 3′ exonuclease and a 5′ exonuclease, respectively, as presented in FIGS. 5 and 6. FIGS. 7A and 7B show the degradation of an oligonucleotide of SEQ ID NO: 1 (SEQ1) after 72 h incubation with 3′ exonuclease at 37° C. FIG. 7A demonstrates that the degradation of 3′ PEGylated oligonucleotide (3′ 400 SEQ1) or 3′ 400- and 5′ 400-PEGylated oligonucleotide (3′ 400+5′ 400 SEQ1) is almost completely reduced (cf. FIGS. 5B, 5C, 5E). FIG. 7B presents that an oligonucleotide of SEQ ID NO: 1, which is linked to PEG2000 at the 3′-end and to PEG400 at the 5′-end (3′ 2000+5′ 400 SEQ1), or is linked to PEG400 at the 3′-end and to PEG2000 at the 5′-end (3′ 400+5′ 2000 SEQ1) is degraded in a much lower rate than an oligonucleotide without PEG (SEQ1) (cf. FIGS. 5D, 5F, 5G). FIGS. 7C and 7D show the degradation of an oligonucleotide of SEQ ID NO: 1 (SEQ1) after 72 h incubation with 5′ exonuclease at 37° C. In FIG. 7C is demonstrated that the degradation rate of an oligonucleotide of SEQ ID NO: 1 linked to PEG400 at the 3′-end of the oligonucleotide (3′ 400 SEQ1) as well as such oligonucleotide linked to PEG400 at the 3′-end and the 5′-end of the oligonucleotide (3′ 400+5′ 400 SEQ1) is clearly reduced in comparison to the oligonucleotide of SEQ ID NO: 1 without PEG (SEQ1) (cf. FIGS. 6C, 6D, 6E). FIG. 7D shows that an oligonucleotide linked to PEG2000 at the 5′-end (5′ 2000 SEQ1), linked to PEG2000 at the 3′-end and PEG400 at the 5′-end (3′ 2000+5′ 400 SEQ1), or linked to PEG400 at the 3′-end and PEG2000 at the 5′-end (3′ 400+5′ 2000 SEQ1) (cf. FIGS. 6B, 6F, 6G).

FIG. 8: This figure shows the chemical reaction of an oligonucleotide having an amino functionalized 5′-end (linker) with PEG-NHS ester resulting in an oligonucleotide linked to PEG at the 5′-end.

FIG. 9: It presents the chemical reactions for the production of 3′5′-PEGylated oligonucleotides using a 3′-aminomodifier including Fmoc for example C7 CPG as described in example 8.

FIG. 10: The figure shows the chemical reactions of an alternative method for the production of 3′5′-PEGylated oligonucleotides using a 5′- and 3′-amino modifier for example a 5′-aminomodifier and 3′-aminomodifier C6 CPG as described in example 10.

FIG. 11: This figure shows the chemical reactions for the production of 3′5′-PEGylated oligonucleotides, wherein the 5′-end of the oligonucleotide is linked to the support using 5′-phosphoramidite building blocks as described in example 11.

Before the invention is described in further detail, it is to be understood that the invention is not limited to the particular embodiments of the invention described below, as variations of the particular may be made and still fall within the scope of the appended claims. It is also to be understood that the terminology employed is for the purpose of describing particular embodiments, and is not intended to be limiting. Instead, the scope of the present invention will be established by the appended claims.

### DEFINITIONS

### Branched

The at least one polymer, more preferred the polyalkylene oxide, even more preferred the polyethyleneglycol, in one embodiment is linear in other embodiments it is branched. Branched means that there is at least one branching in the respective polymer comprising that there are several branches in the molecule. Branches in the polymer have the advantage that the molecule is more compact and disadvantages of long linear polymers, such as masking the oligonucleotide, are compensated.

The term “branched” further comprises polymers such as PEG which are combined via a linker, e.g., a Lys core to form a “pseudo-branched” polymer. In this case the polymer represents a monomere of a multi-polymer complex.

### Diseases and Disorders

The conjugates or compositions of the invention act as novel therapeutic agents for controlling, treating and/or preventing one or more of cellular proliferative and/or differentiative diseases or disorders, diseases or disorders associated with bone metabolism, immune, hematopoietic, cardiovascular, liver, kidney, muscular, hematological, viral, pain, neurological and/or metabolic diseases or disorders, in particular disorders or diseases associated with undesired TGF-beta signaling. Cellular proliferative and/or differentiative diseases or disorders include for example cancer, e.g., carcinoma, sarcoma, metastatic or hematopoietic neoplastic diseases or disorders such as leukemias. As used herein, the term “cancer”, or “carcinoma” means new and abnormal growth or formation of tissue and/or blood cells in the body of a organism also comprised by the term “neoplasm”. The term “cancer”, “carcinoma” and “neoplasm” include malignancies of the various organ systems for example such affecting brain, eye, lung, breast, thyroid, lymphoid, gastrointestinal, and genito-urinary tract. The conjugates or compositions of the invention are particularly designed to target genes associated with particular diseases or disorders.

### Conjugate

A conjugate or compound in the context of this application refers to an oligonucleotide, protein and/or peptide, which is for example an immunostimulator, linked with at least one polymer. In a conjugate or compound for example comprising polyalkylen oxide and an oligonucleotide, a protein and/or a peptide, wherein the polyalkylen oxide is linked to the oligonucleotide, protein and/or peptide, the molecular weight of the polyalkylen oxide is at least 200 Da. Preferably, at least one polyalkylen oxide is linked to the 5′-end of the oligonucleotide and/or at least one polyalkylen oxide is linked to the 3′-end of the oligonucleotide, and/or at least one polyalkylen oxide is linked to a phosphate group, a sugar moiety, and/or a base of the oligonucleotide in such conjugates or compounds.

PEGylating means linking at least one polyethyleneglycol (PEG) to another molecule, in the context of this invention to an oligonucleotide, protein and/or peptide, e.g., an immunostimulator. Preferably the polyethyleneglycol is methylated (mPEG).

### Linkage

A linkage also referred to as link between two molecules e.g., a polymer and an oligonucleotide, protein and/or a peptide is any kind of covalent connection between at least two molecules. The covalent linkage is preferably supported by additional interactions of the molecules forming the conjugate such as non-covalent, intermolecular forces, e.g., van-der-Waals forces. The molecules of the conjugate, i.e., the oligonucleotide, protein and/or peptide, and the polymer are preferably linked, i.e., connected by linkers.

### Linker

Linker synonymously also referred to as cross-linker in the context of this invention refers to any chemical substance able to bind at least two molecules, e.g., an oligonucleotide, protein and/or peptide with at least one polymer. Linkers include zero length linkers, homobifunctional crosslinkers, heterobifunctional cross linkers and the like. Different linkers are usable and combinable, respectively, in a conjugate or compound of the invention, i.e., different linkers are directly combined or different linkers are used to link one or more oligonucleotides and/or one or more proteins and/or one or more peptides. Preferably a linker has the function of a spacer. In particular, the polymer is the linker, combining oligonucleotides with each other, or an oligonucleotide with a protein, such as a receptor, and/or a peptide, such as a receptor fragment. The term “linker” further comprises a linker comprising or consisting of a linker and an oligonucleotide, a protein and/or a peptide, or comprises a linker comprising or consisting of a linker and a polyalkylen oxide such as PEG.

### Spacer

Spacer in the context of this invention is any molecule that connects two or more molecules in a certain distance to each other. In some embodiments the linker used in this invention is also an spacer, but the spacer might be used additionally with a linker in the molecule. In certain embodiments the spacer substitutes at least one nucleotide building block or parts of the nucleotide building block. In other embodiments the spacer substitutes at least one monomer of a polymer block or parts of that monomer. Compounds comprising spacers such as 5′-aminomodifier C3, -C5, -C6, -C7 are also within the scope of this invention. Preferably, the conjugate or compound of the invention comprises one or more spacer, wherein the type of spacers is identical or different. Such a spacer is for example a maleimide spacer, an alkyl spacer, a peptide spacer, a glucuronide spacer, a nonionic or polyionic spacer etc.

### Oligonucleotide

Size of an oligonucleotide is at least 5 nucleotides, preferably between 5 and 70 nucleotides, preferred between 10 and 60 nucleotides, more preferred between 10 and 40 nucleotides, even more preferred between 12 and 25 nucleotides, and most preferred between 12 and 20 nucleotides. In particular the oligonucleotides comprise 10, 11, 12, 13, 14, 15, 16, 17, 18, 19, 20, 21, 22, 23, 24, or 25 nucleotides. The oligonucleotide is generated in any manner, preferably by chemical synthesis. DNA replication, reverse transcription, or a combination thereof.

Preferably, the oligonucleotides in the context of the invention comprise any type of oligonucleotide including oligonucleotides having one or more modifications, e.g., an additional functional group for example an amino or a hydroxy group e.g., at one or both ends of the oligonucleotide. These groups allow for example the reaction with a polymer leading to an oligonucleotide polymer conjugate, e.g., a PEGylated oligonucleotide.

In the context of this invention, the term “oligonucleotide” refers to an oligomer of ribonucleic acid (RNA) or deoxyribonucleic acid (DNA) or mimetics thereof also referred to as nucleotide building block-polymers. The term “oligonucleotide” comprises single and double stranded RNA or DNA. In case of double stranded RNA or DNA the strands are blunt end or with overhanging ends, wherein one strand has an overhang on one side of the other strand or on both ends, or one strand has an overhang on one side and the other strand has an overhang on the other side.

The term “oligonucleotides” further comprises aptamers and/or spiegelmers. Aptamers are nucleic acid molecules, comprising at least 25 nucleotides, preferably 10 to 100 nucleotides, more preferably 25 to 75 nucleotides, even more preferably 30 to 70 nucleotides, most preferred between 40 to 60 nucleotides, having specific binding affinity to molecules through interactions other than classic Watson-Crick base pairing. Aptamers are engineered through repeated rounds of in vitro selection or equivalently, SELEX (systemic evolution of ligands by exponential enrichment) to bind to various molecular targets such as small molecules, proteins, nucleic acids, even cells, tissues and organisms, preferably with affinities in the nanomolar to the picomolar range. Aptamers are for example engineered completely in a test tube, are readily produced by chemical synthesis, possess desirable storage properties, and elicit little or no immunogenicity in therapeutic applications. Moreover, aptamers are combinable with ribozymes to self-cleave in the presence of their target molecule.

Spiegelmers are developed on basis of aptamers; they consist of nucleotides having the L-form leading to a high resistance against nucleases. Hence, spiegelmers have all the diversity characteristics of aptamers as well as their binding characteristics, preferably in the low nanomolar to picomolar range, but possess a structure that prevents enzymatic degradation. Aptamers and spiegelmers bind both to extracellular and intracellular molecules such as a receptor or its ligand, to a transcription factor, or a lipid-containing molecule.

Furthermore, the term “oligonucleotide” comprises RNAi, shRNA, and microRNA (miRNA). RNAi is a mechanism for RNA-guided regulation of gene expression in which double-stranded ribonucleic acid inhibits preferably the expression of genes with complementary nucleotide sequences. The RNAi pathway is initiated by the enzyme dicer, which cleaves double-stranded RNA to short double-stranded fragments preferably of 15 to 35 base pairs, more preferably of 20 to 30 base pairs, and most preferably 20 to 25 base pairs. One of the two strands of each fragment, which is the “guide strand”, is incorporated into the RNA-induced silencing complex (RISC) and base-pairs with a complementary sequence. The most-well studied effect of RNAi is post-transcriptional gene silencing, which occurs when the guide strand base pairs with a mRNA and induces degradation of the mRNA for example by argonaute, a catalytic component of the RISC complex. The short fragments are known as small interfering or silencing RNA (siRNA), which are preferably perfectly complementary to the gene which is to be suppressed. In addition to their role in RNAi pathway, siRNA also act in RNAi-related pathways, e.g., as an antiviral mechanism or in shaping the chromatin structure of a genome. Similar to siRNA, microRNA (miRNA), which are single-stranded RNA molecules of 15 to 30 nucleotides, preferably 20 to 30 nucleotides, and most preferably 21 to 23 nucleotides, regulate preferably gene expression. miRNA is encoded by genes that are transcribed from DNA, but not translated into protein (non-coding RNA). Instead the miRNA is processed from primary transcripts known as pre-miRNA preferably to short stem-loop structures such as pre-miRNA and finally to functional miRNA. A further type of RNA preferably involved in gene silencing is short hairpin RNA (shRNA). shRNA is RNA which makes a tight hairpin turn that is preferably suitable to silence gene expression via RNAi. shRNA uses a vector introduced into cells and utilizes a promoter, preferably the U6 promoter, to ensure that the shRNA is expressed. The vector is preferably passed on to daughter cells, allowing the gene silencing to be inherited. The shRNA hairpin structure is preferably cleaved by the cellular machinery preferably into siRNA, which is then bind to RISC and starts the mechanism as described above.

Additionally, the term oligonucleotide comprises CpG oligonucleotides. CpG motifs induce Toll-like receptor mediated immune response by simulating bacterial DNA. The CpG oligonucleotide preferably activates immune cells such as dendritic cells and B lymphocytes, and stimulates NF-κB. CpG oligonucleotides are in general not a target sequence specific approach.

Moreover, the term oligonucleotide comprises a decoy oligonucleotide, also known as “decoy”, which is preferably a double-strand oligonucleotide bearing a consensus binding sequence for example of a specific transcription factor for manipulating gene expression.

The oligonucleotides of the present invention preferably hybridize with mRNA of a molecule negatively influencing a physiological and/or biochemical effect in a cell or hybridize with mRNA of the receptor of the molecule. More preferably, the oligonucleotides of the invention hybridize with mRNA of TGF-beta1, TGF-beta2, TGF-beta3, VEGF, IL-10, c-jun, c-fos, Her-2. MIA, and/or its receptor.

### Nucleotide Building Block—Modification

Oligonucleotides comprise nucleotide building blocks composed of base, sugar, and phosphate moiety. Oligonucleotides include oligonucleotides having non-naturally occurring oligonucleotide building blocks with similar function. Naturally occurring nucleotides as well as non-naturally occurring nucleotides, modifications of these nucleotides at the base, the sugar or the backbone as well as spacers instead of a at least one nucleotide are also referred to as nucleotide building block. Modifications of an oligonucleotide are for example phosphorothioate, methylphosphonate, phosphoramidate, or 2′-modifications of the sugar (e.g., 2′-O-methyl oligonucleotide, 2′-O-methoxy-ethyl oligonucleotide, or 2′-deoxy-2′-fluoro oligonucleotide).

The oligonucleotide preferably comprises at least 5 nucleotide building blocks, preferably 5 to 120 nucleotide building blocks, more preferably 8 to 30 nucleotide building blocks, even more preferably 10 to 28 nucleotide building blocks, even more preferred 12 to 26 nucleotide building blocks, most preferred 10, 11, 12, 13, 14, 15, 16, 17, 18, 19, 20, 21, 22, 23, 24, 25, or 26 nucleotide building blocks.

The term nucleotide building block comprises nucleotides composed of naturally-occurring nucleobases, sugars and covalent internucleoside (backbone) linkages, each of these also referred to as portions, as well as oligonucleotides having non-naturally-occurring portions which function similarly, e.g. hybridizing with the same mRNA of a selected target. In one embodiment the base is modified or substituted by a similar molecule. Similar bases are those molecules that are also able to support the hybridization to the mRNA or at least do not affect the hybridization in a negative way. In some embodiments at least one base portion is substituted with a spacer.

In other embodiments the sugar moiety of the nucleotide building block is modified or substituted by another group, structure, or moiety. Examples for sugars are arabinose, xylose, lyxose, allose, altrose, glucose, mannose, gulose, idose, galactose, talose or stabilized modifications of those sugars. In some embodiments the sugar is substituted by a spacer.

In other embodiments the internucleoside linkage, also referred to as linkage between two nucleotide building blocks is not a phosphorodiester but another group, structure, or moiety.

Such oligonucleotides with at least one modified nucleotide building block are often preferred over native forms because of desirable properties such as enhanced cellular uptake, enhanced affinity for nucleic acid target, increased stability in the presence of nucleases and/or enhanced therapeutic effectiveness.

Oligonucleotides, having a modified nucleotide building block, further comprise for example peptide nucleic acid (PNA), locked nucleic acid (LNA), and morpholinos as shown in the following:

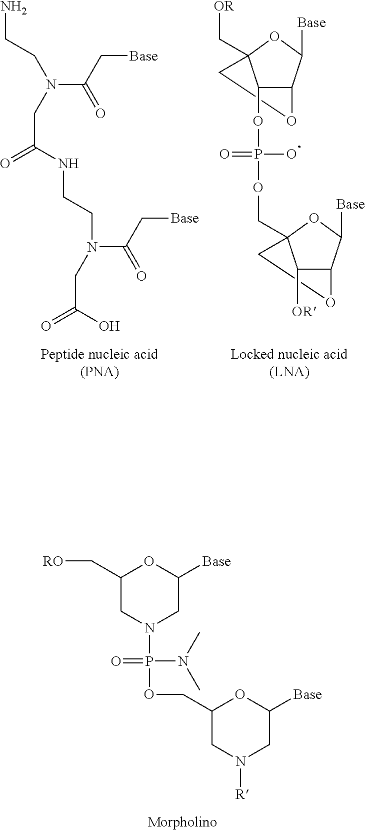

PNA is a chemical similar to DNA and RNA, and is generally artificially synthesized. The PNA's backbone is composed for example of repeating N-(2-aminoethyl)-glycine units linked by peptide bonds. The various purine and pyrimidine bases are preferably linked to the backbone by methylene carbonyl bonds. Since the backbone of PNA contains no charged phosphate groups, the binding between PNA/DNA strands is in general stronger than between DNA/DNA.

LNA is a modified RNA, wherein the ribose moiety of an LNA nucleotide is modified with an extra bridge connecting the 2′ and 4′ carbons. The bridge “locks” the ribose in the 3′-endo structural conformation, which is often found in the A-form of DNA or RNA. LNA is combinable with DNA or RNA bases in an oligonucleotide.

Morpholino oligonucleotides are an antisense technology used to block access of other molecules to specific sequences with nucleic acid. Morpholinos block small (about 25 base) regions of the base-pairing surface of RNA or DNA.

### Protein

Proteins are consisting of at least 120 amino acids, wherein the term “amino acid” does not only comprise naturally occurring amino acids, but also chemically modified amino acids. The amino acids are either isolated from natural sources or biotechnologically modified microorganisms. The proteins are either isolated or synthesized for example via the Merrifield method. Such proteins are for example enzymes, antibodies or receptors or fragments thereof.

The term protein according to the present invention comprises glycoproteins, which are proteins that contain oligosaccharide chains (glycans) covalently attached to their polypeptide backbones. Preferably, the oligosaccharide is connected to the amide nitrogen of the side chain of Asp (N-glycosylation), or to the hydroxyloxygen of the side chain of hydroxylysine, hydroxyproline, serine, or threonine (O-glycosylation).

### Peptide

The term “peptide” comprises dipeptides, oligopeptides and polypeptides. Dipeptides, comprising two amino acids, are for example Carnosin, Anserin, Homoanserin, Kyotorphin, Balenin, Aspartam, Glorin, Barettin, or Pseudoprolin. Oligopeptides comprise only a low number of amino acids, e.g., 3 to 15 amino acids, preferably 5 to 15 amino acids, and more preferably 8 to 15 amino acids. Preferably oligopeptides comprise 3, 4, 5, 6, 7, 8, 9, 10, 11, 12, 13, 14, or 15 amino acids. Polypeptides comprise between 15 and 120 amino acids, preferably between 30 and 100 amino acids, more preferably between 60 and 120 amino acids, most preferably between 90 and 100 amino acids. In particular, peptides comprise, 15, 20, 25, 30, 35, 40, 45, 50, 55, 60, 65, 70, 75, 80, 85, 90, 95, 100, 110, or 120 amino acids. Such proteins are for example enzymes, antibodies or receptors or fragments thereof.

Aptamers are as above mentioned nucleic acid molecules. Alternatively, aptamers are peptide or protein molecules. A typical aptamer has at least 5 kDa, preferably 5 to 25 kDa, more preferably 10 to 20 kDa, and most preferably 10 to 15 kDa in size. Aptamers are preferably characterized by high specificity and affinity, biological efficacy, and excellent pharmacokinetic properties. Aptamers are for example suitable as molecular “chaperones”, increasing the specificity of another molecule to a given target by linking the molecule to an aptamer with high binding affinity to a target. Such molecule is for example a cytotoxic agent, e.g., chemotoxins such as tubulin stabilizers/destabilizers, anti-metabolites, purine synthesis inhibitors, nucleoside analogs, and DNA-modifying agents, a toxin, e.g., a radioisotope, receptor tyrosine kinases, EGFR, Her2 new, PSMA, and Muc1, or a chemotherapeutic agent.

### Protecting Group (Protective Group)

In preferred embodiments of the invention one or more protecting groups, also called protection groups, are used in the production of the conjugate or compound. In general, protecting groups render chemical functionalities inert to specific reaction conditions and are appended to and/or removed from such functionalities in a molecule without substantially damaging the remainder of the molecule. The term “protected” means that the indicated moiety has a protecting group appended thereon.

Hydroxy protecting groups are for example t-butyl, t-butoxymethyl, methoxymethyl, tetrahydropyranyl, 1-ethoxyethyl, I-(2-chloroethoxy)ethyl, 2-trimethylsilyl ethyl, p-chlorophenyl, 2,4-dinitrophenyl, benzyl, 2,6-dichlorobenzyl, diphenylmethyl, p, p′-dinitrobenzylhydryl, triphenylmethyl, trimethylsilyl, triethylsilyl, t-butyldimethylsilyl, t-butyldiphenylsilyl, triphenylsilyl, benzoylformate, acetate, chloroacetate, trichloroacetate, trifluoroacetate, pivaloate, benzoate, p-phenylbenzoate, 9-fluorenylmethyl carbonate, mesylate and tosylate.

Amino protecting groups are for example monomethoxytrityl (MMT), 2-trimethylsilylethoxycarbonyl (Teoc), 1-methyl-1-(4-biphenyl)ethoxycarbonyl (Bpoc), t-butoxycarbonyl (BOC), allyloxycarbonyl (Alloc), 9-fluorenylmethyloxycarbonyl (Fmoc), benzyloxycarbonyl (Cbz); amide protecting groups such as formyl, acetyl, trihaloacetyl, benzoyl, and nitrophenylacetyl; sulfonamide protecting groups such as 2-nitrobenzenesulfonyl, or imine and cyclic imide protecting groups such as phthalimido and dithiasuccinoyl.

Carbonyl protecting groups are for example acetals, ketals, acylals, or dithianes. Carboxylic acid protecting groups are for example methyl esters, benzyl esters, tert-butyl esters, or silyl esters.

Equivalents of the protecting groups such as hydroxy protecting groups, amino protecting groups, carbonyl protecting groups, carboxylic acid protecting groups or amino protecting groups are also encompassed by the conjugates or compounds and the methods of their production.

Moreover, the production of oligonucleotide, protein and/or peptide conjugates according to the invention comprises orthogonal protection, which is a strategy allowing the deprotection of multiple protective groups one at the time each with a dedicated set of reaction conditions without affecting the other. In particular, orthogonal protection is used for the production of proteins and peptides as well as their conjugates of the invention. Preferably, orthogonal protection is used for high selective conjugation of an oligonucleotide, a protein and/or a peptide with a polymer, in particular polyalkylen oxide such as PEG.

Depending on the protecting group for example more acidic or more basic conditions allow to remove the protecting group from the oligonucleotide, protein or peptide.

### Solid Phase

In the context of this invention a solid phase is any inert material on which the synthesis of an oligonucleotide, protein, peptides, of any polymer if performable. A preferred embodiment of a solid phase is a controlled pore glass. For example controlled means that reactive groups of the solid phase are neutralized.

### Immunostimulator

An immunostimulator according to this invention is any substance inducing the function of immune cells and/or the immune system to enhanced abilities directly or indirectly reducing or inhibiting the tumor cell growth and/or inducing cell death of unwanted neoplasms in a pharmaceutical acceptable carrier.

In one embodiment the oligonucleotide, the protein and/or the peptide such as an immunostimulator is selected from the group of chemokines, including but not limited to lymphotactin, interleukin 1, interleukin 2, interleukin 6, interleukin 12, interferon gamma, and/or immune cell attracting substances.

In yet another embodiment the oligonucleotide, the protein and/or the peptide such as an immunostimulator is selected from the group of viruses and/or parts of viruses, including retroviruses, adenoviruses, papillomaviruses. Epstein-Barr-viruses and viruses that are non-pathogenic including Newcastle-Disease virus, Cow-pox-virus.

In another embodiment the oligonucleotide, the protein and/or the peptide, e.g., an immunostimulator is selected from the group of autologous, heterologous MHC-Molecules, molecules involved in antigen processing, molecules involved in antigen presentation, molecules involved in mediating immune cell effects, molecules involved in mediating immune cell cytotoxic effects, molecules involved in antigen transportation, co-stimulatory molecules, peptides enhancing recognition by immune cells and/or cytotoxic effects of immune cells.

In yet another embodiment the protein or peptide, for example an immunostimulator is a protein or peptide enhancing the recognition of unwanted neoplasms by immune cells and/or cytotoxic effects of immune cells containing one or more mutations and/or amino acid substitutions of the ras proteins, the p53 protein, the EGF-receptor protein, fusion peptides and/or fusion proteins, the retinoblastoma protein, proteins coded by oncogenes and/or protooncogenes and/or proteins coded by anti-oncogenes and/or tumor suppressor genes.

In yet another embodiment the protein or peptide such as an immunostimulator is a protein or peptide enhancing the recognition of unwanted neoplasms by immune cells and/or cytotoxic effects of immune cells containing one or more mutations and/or amino acid substitutions caused by gene rearrangements and/or gene translocations.

In yet another embodiment the protein or peptide such as an immunostimulator is a protein or peptide enhancing the recognition of unwanted neoplasm by immune cells and/or cytotoxic effects of immune cells derived from proteins differing in the target cell by one or more amino acids from the proteins expressed by other cells in the same organism.

In yet another preferred embodiment the protein or peptide such as an immunostimulator is a protein or peptide enhancing the recognition of unwanted neoplasm by immune cells and/or cytotoxic effects of immune cells derived from viral antigens and/or coded by viral nucleic acids.

In yet another embodiment the protein or peptide such as an immunostimulator is a protein or peptide derived from proteins expressed in a diseased organ but not in the nervous system, muscle, hematopoetic system or other organs essential for survival. Diseased organs are e.g. prostate, ovary, breast, melanin producing cells and the like.

In yet another embodiment the protein or peptide, e.g., an immunostimulator is a protein or peptide containing one or more amino acids differing between a protein in the target cell from the other cells within an organism, tumor cell extracts, tumor cell lysates and/or adjuvants.

In yet another embodiment the immunostimulator is a fusion cell of a dendritic and a tumor cell or is a dendritic cell. These fusion cells are hybridoma cells derived from a mixture of dendritic cells and tumor cells. Dendritic cells are generated e.g. by treatment of PBMC with GM-CSF and IL-4 or a mixture of GM-CSF, IL-4 and IFN-γ or FLT-3 ligand. Fusion of dendritic cells with tumor cells can be achieved e.g. using PEG (polyethyleneglycol) or electrofusion (Hayashi, T., et al. 2002, Parkhurst, M. R. 2003, Phan, V. 2003).

In yet another preferred embodiment the protein and/or the peptide for example an immunostimulator is an antagonist of factors negatively influencing the function of the immune system. These factors are e.g. TGF-beta1, -2, or -3 (transforming growth factor beta1, -2, or -3), VEGF (vascular endothelial growth factor), PGE₂ (prostaglandin E₂), IL-10 (interleukin 10), or MIA, or fragments thereof.

In yet another embodiment the oligonucleotide, the protein and/or the peptide such as an immunostimulator is a vaccine.

Vaccines according to this invention comprise but are not limited to substance in a pharmaceutical acceptable carrier selected from the group of whole (irradiated) tumor cells, ASI (active specific immunization) with e.g. Newcastle Disease Virus (NDV) modified tumor cell vaccine (Schneider, T. et al. 2001), tumor cell lysates.

In one preferred embodiment the vaccines are peptides combined with cytokines (e.g. IL-2, IL-12, GM-CSF) or peptides combined with adjuvants (e.g. incomplete Freund's adjuvant, QS21).

In yet another embodiment of vaccination a recombinant virus construct that encodes carcinoma antigen(s) is part of e.g. adenovirus, vaccinia, fowlpox and/or avipox.

In yet another embodiment the vaccine is naked DNA or RNA encoding carcinoma antigen(s).

In yet another embodiment the vaccine comprises or consists of dendritic cells, dendritic cells loaded with peptides derived from carcinoma antigens, dendritic cells transfected with recombinant viruses or RNA. DNA and/or cDNA encoding different tumor antigens, dendritic cells pulsed with tumor lysates and/or dendritic cells fused with whole tumor cells. For further vaccines see also Jager, E. et al. 2003.

In a preferred embodiment of this invention the oligonucleotide, the protein and/or the peptide for example an immunostimulator is an antagonist of factors negatively influencing the function of the immune system.

An antagonist as used herein is any substance inhibiting any physiological and/or biochemical effect, for example inhibiting the production of e.g. a cytokine and/or the effect of cytokines. Examples for cytokines negatively influencing the immune systems are e.g. TGF-beta such as TGF-beta1. TGF-beta2, or TGF-beta3 VEGF, IL-10, PGE-E₂, or MIA. The inhibition in one embodiment works by binding the cytokine to a binding protein, to a receptor or to a part of this receptor, by binding the cytokine with an antibody, a low molecular substance inhibiting the cytokine or its production, or by inhibiting the signal pathway of said cytokine, e.g. by inhibiting the receptors of these cytokines or any other link downstream in the activation cascade of cytokines.

TGF-beta (transforming growth factor beta) in the context of this invention comprises all subclasses of TGF-beta, preferred subclasses are TGF-beta 1, TGF-beta 2, and TGF-beta 3.

More details are given for example for the preferred embodiment of TGF-beta antagonists such as TGF-beta1, TFG-beta2, or TGF-beta3 antagonists, which are transferable to the cytokines described above as well.

Preferably, antagonist of the immune system as used herein is any substance or method inhibiting the activity of the immune system.

“Low molecular substances” or “small molecules” herein comprise substances with a molecular weight of less than about 10 kg/mol and more than about 1 g/mol, preferably less than about 1 kg/mol of organic or inorganic origin.

In a preferred embodiment the immunostimulator of the pharmaceutical composition of this invention is a TGF-beta antagonist, preferably a TGF-beta1. TGF-beta2, or TGF-beta3 antagonist.

In the context of this invention a TGF-beta antagonist is any oligonucleotide, protein and/or peptide or any substance inhibiting the function of TGF-beta in the meaning that any effect that is induced by TGF-beta is inhibited.

In preferred embodiments the TGF-beta antagonist is an oligonucleotide, protein and/or peptide or another substance inhibiting the production of TGF-beta, is an oligonucleotide, protein and/or peptide or another substance binding TGF-beta and/or is an oligonucleotide, protein and/or peptide or another substance inhibiting the function of TGF-beta downstream its activation cascade. TGF-beta antagonists are for example anti-TGF-beta antibodies, small molecule inhibitors of TGF-beta, or Smad inhibitors as described in Wojtowicz-Praga (2003) herein incorporated by reference.

Examples for TGF-beta antagonists are given in Example 15.

In one embodiment of TGF-beta antagonists or inhibitors inhibiting the production of TGF-beta, the antagonist or inhibitor is an oligonucleotide and/or its active derivative hybridising preferably with a specific area of the messenger RNA (mRNA) of TGF-beta and/or the DNA encoding TGF-beta and thereby inhibiting the production of TGF-beta.

In yet another embodiment TGF-beta antagonists or inhibitors, e.g., TGF-beta1. TGF-beta2, or TGF-beta3 antagonists or inhibitors, are receptors and/or parts of it binding TGF-beta and in that way inhibiting the function of TGF-beta.

In yet another embodiment the TGF-beta antagonist or inhibitors is an antibody and/or parts of it binding TGF-beta and by this inhibiting the function of TGF-beta, in particular TGF-beta1. TGF-beta2, or TGF-beta3, or fragments thereof. Those antibodies are commercially available, see e.g. R & D Systems, Inc. The production of such antibodies is well known in the art. Animals for example chicken, mice, rabbits, goats, are immunized with purified human TGF-beta, particularly TGF-beta1. TGF-beta2, or TGF-beta3. After immunization, IgY is purified for example via affinity chromatography as described for example by Cooper, N. M. (1995). In yet other embodiments the TGF-beta antibodies are modified, e.g., biotinylated.

In a more preferred embodiment the TGF-beta antibodies are humanized antibodies. For example humanized TGF-beta1, and TGF-beta2 antibodies as described in Carrington et al. (2000 and 2006).

In yet another embodiment the TGF-beta antagonist or inhibitors is an oligonucleotide, a protein and/or a peptide binding to TGF-beta, and by this inhibiting the function of TGF-beta. Preferred embodiments of the peptides are e.g. latency-associated peptides, which are suitable to inhibit all isoforms of TGF-beta for example TGF-beta 1, TGF-beta 2 and TGF-beta 3.

In another embodiment the TGF-beta inhibitor is an oligonucleotide, a protein, a peptide or a small molecule inhibiting the function of the TGF-beta receptor, acting extracellularly or intracellularly.

In yet other embodiments the TGF-beta antagonists or inhibitors comprise oligonucleotides, proteins, peptides, antibodies and/or small molecules, which inhibit the TGF-beta activity by inhibiting any link downstream of the TGF-beta cascade of activation.

In a preferred embodiment of this invention the antagonist or inhibitor of a peptide, cytokine and/or receptor is an oligonucleotide according to this invention.

In yet another embodiment the antagonist or inhibitor inhibiting the production of TGF-beta is for example a peptide, a peptide of less than 100 kg/mol, a peptide being part of TGF-beta, a protein, a protein that is not an antibody, and/or a small molecule, e.g. tranilast (N-[3,4-dimethoxycinnamoyl]-anthranilic acid) (Wilkenson, K. A. 2000).

In one embodiment the protein or peptide being part of TGF-beta is an amino acid sequence of TGF-beta 1, TGF-beta2 and/or TGF-beta3 which are published for example in Mittl (1996) herein incorporated by reference.

In one preferred embodiment a peptide comprises the 112 amino acids starting counting from the end of the TGF-beta1. TGF-beta2 or TGF-beta 3 protein, i.e., the last 112 amino acids of one of these proteins. The start of those peptides is after the RXXR motif ending 113 amino acids before the end of the TGF-beta1, TGF-beta2 or TGF-beta3 protein, wherein R is the amino acid arginin, and XX represents any amino acid or is even no amino acid.

In yet other embodiments a peptide being part of TGF-beta comprises or is part of one or more of the sequences presented in example 9, comprising one to all amino acids of this peptide. In other embodiments a preferred peptide, e.g., a TGF-beta peptide, comprises or consists of about 1-100 amino acids, about 2-50 amino acids, about 3-30 amino acids or about 5-20 amino acids. In further preferred embodiments the peptide is part of any of the amino acid sequences of a TGF-beta protein as described above comprising or consisting of about 1-50 amino acids, about 1-40, about 2-30, about 3-25, about 4-18, about 5-15 or about 6-12 amino acids.

In yet other embodiments preferred amino acid sequences, i.e., peptides are those presented in example 9 for TGF-beta1. TGF-beta2 and TGF-beta3 with the respective numbers 1-21.

Amino acids replaced conservatively, also referred to as conservative analogs or active derivatives of peptides in the context of this invention, means replacing at least one amino acid of a peptide or protein. Preferably at least one acid amino acid (e.g., glutaminic acid (E), asparaginic acid (D)) is replaced by the respective other amino acid, accordingly at least one basic amino acids is replaced by another basic amino acid, at least one amino acid with a polar group (—OH, —SH, —CONH₂) is replaced by another amino acid with a polar group and/or amino acids with pure carbon side chains such as aliphatic side chains are replaced by another amino acid with pure carbon side chains. Peptides and/or proteins conservatively replaced with amino acids are still in the scope of this invention.

In yet other embodiments of the proteins or peptides of the invention at least one of the basic amino acids selected from the group of histidine (H), lysine (K) and arginine (R) is substituted by another basic amino acid selected from this group without loosing its TGF-beta antagonizing effects.

In yet other embodiments of the proteins or peptides of the invention at least one of the acid amino acids selected from the group of glutaminic acid (E) and asparaginic acid (D) is substituted by its counterpart of this group without loosing its TGF-beta antagonizing effects.

The peptides that are part of TGF-beta and wherein some amino acids are replaced conservatively compared to their sequences presented in example 9 are also referred to as analogs of TGF-beta1, TGF-beta2 and/or TGF-beta3.

In some embodiments in the analogs of TGF beta1, TGF-beta2 or TGF-beta3 1 to about 30%, about 2% to about 20%, about 3% to about 15%, 4% to about 12% or about 5% to about 10% of the amino acids are replaced conservatively.

In another embodiment the oligonucleotides, proteins and/or peptides of the invention are either used alone or in combination with a chemotherapeutic agent. In yet another embodiment the oligonucleotides, proteins and/or peptides are used for preparing a pharmaceutical composition with a pharmaceutically acceptable carrier. In yet another embodiment the oligonucleotides, proteins and/or peptides are comprised by a pharmaceutical composition for the treatment of neoplasms or solid tumors and in yet another embodiment these peptides are used for a method treating neoplasms or solid tumors according to this invention, more preferred glioma, astrocytoma and/or glioblastoma.

In one embodiment the oligonucleotide, the protein and/or the peptide such as an immunostimulator is an oligonucleotide, protein and/or peptide, that is linked with at least one polymer, more preferred polyalkylene oxide, most preferred with polyethyleneglycol.

Yet another aspect of this invention is a conjugate and compound, respectively, comprising an oligonucleotide, a protein and/or a peptide linked with at least one polymer as described herein.

The conjugation with the polymer is anywhere within the molecule, i.e., the oligonucleotide, the protein or the peptide. With regard to the oligonucleotide, the linkage is preferably at the 3′ and/or 5′ end of the oligonucleotide. In a preferred embodiment, polymers of same or different type and/or molecular weight are linked to the 3′ or/and to the 5′ end of the oligonucleotide. The term 3′ and 5′ end, respectively, refers to the carbon atom of the sugar moiety of the oligonucleotide or the nucleotide building block. If the sugar is substituted by another molecule, this term is used in an analogous way, which means that it is looked upon the oligonucleotide or the nucleotide building block in that way that there would be a sugar moiety. Preferably, the polymer for example the polyalkylen oxide such as PEG linked to the 5′-end of the oligonucleotide has the 1.5- to 100-fold molecular weight of the polyalkylen oxide linked to the 3′-end of the oligonucleotide, or the polyalkylen oxide linked to the 3′-end of the oligonucleotide has the 1.5- to 100-fold molecular weight of the polyalkylen oxide linked to the 5′-end of the oligonucleotide, or the polyalkylen oxide linked to the 5′-end or 3′-end of the oligonucleotide has the 1.5- to 100-fold molecular weight of the polyalkylen oxide linked to the phosphate group, the sugar moiety and/or any base of the oligonucleotide, or the polyalkylen oxide linked to the phosphate group, the sugar moiety and/or any base of the oligonucleotide has the 1.5- to 100-fold molecular weight of the polyalkylen oxide linked to the 5′-end or 3′-end of the oligonucleotide. More preferred, the molecular weight of the polymer for example polyalkylen oxide such as PEG, which is linked to the 5′-end of the oligonucleotide is 200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000 Da and the molecular weight of the polyalkylen oxide linked to the 3′-end of the oligonucleotide is 200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000 Da, or the molecular weight of the polyalkylen oxide linked to the 3′-end of the oligonucleotide is 200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000 Da and the molecular weight of the polyalkylen oxide linked to the 5′-end of the oligonucleotide is 200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000 Da, or the molecular weight of the polyalkylen oxide linked to the 5′-end and/or the 3′-end of the oligonucleotide is 200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000 Da and the molecular weight of the polyalkylen oxide linked to the phosphate group, the sugar moiety and/or any base of the oligonucleotide is 200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000 Da, or the molecular weight of the polyalkylen oxide linked to the phosphate group, the sugar moiety and/or any base is 200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000 Da and the molecular weight of the polyalkylen oxide linked to the 5′-end and/or the 3′-end of the oligonucleotide is 200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000 Da.

Further possibilities for linking the at least one polymer with the oligonucleotide are for example linking it to the phosphorodiester group, to any other O-atom of the sugar moiety or to an active group of the respective substitute. In other embodiments the polymer is linked with an active group of the base, its modification, its substitute or with a linker and/or a spacer. The polymer can also be linked to a spacer within the compound.

Preferably, one conjugate or compound comprises one or more polymers linked to the oligonucleotide, protein and/or peptide. The linkage of several polymers induces for example better pharmacological activity, less side effects, better cellular uptake, improved hybridization, improved halflife time, and/or reduced toxicity.

The oligonucleotide or nucleotide building block linked with the at least one polymer comprises or consists of about 8 to about 30 nucleotides. Preferred oligonucleotides are oligonucleotides that hybridize with mRNA coding for molecules preferably relevant in the process of inhibiting the synthesis and/or the function of molecules suppressing and/or downregulating and/or negatively affecting cellular processes, in particular the immune response.

In preferred embodiments these molecules are TGF-beta1, TGF-beta2, TGF-beta3, VEGF, interleukin-10, c-jun, c-fos, c-erbB2, their respective receptors and/or the prostaglandin E2 receptor. Further preferred embodiments of oligonucleotides are given in the sequence listing, in examples 6 and 7 or are oligonucleotides published in WO 94/25588, WO 95/17507, WO 95/02051, WO 98/33904, WO 99/63975, WO 01/68146, WO 01/68122, WO 03/06445, WO 2005/014812, WO 2005/059133, WO 2005/084712 herein incorporated by reference.

Preferred oligonucleotides that are linked with at least one polymer comprise or consist of at least one of SEQ ID NO 1 to 435, or comprise or consist of at least one of the sequences of examples 7 and 8.

Especially preferred are SEQ ID NO. 1, 2, 3, 28 and 37. Mostly preferred are the oligonucleotides comprising or consisting of SEQ ID No.: 1 (CGGCATGTCTATTTTGTA) and/or SEQ ID No.: 28 (CTGATGTGTTGAAGAACA).

In yet other embodiments the oligonucleotide is a “chimeric” oligonucleotide. In the context of this invention chimeric oligonucleotides are oligonucleotides, which contain at least one chemically region wherein at least one portion of the nucleotide building block is modified. These oligonucleotides show for example increased resistance to nuclease degradation, increased cellular uptake, and/or increased binding affinity for the target nucleic acid.

In one embodiment the oligonucleotide or the nucleotide building block includes for example sugar moieties that are covalently attached to low molecular weight organic groups other than a hydroxyl group at the 3′ and/or 2′ position and other than a phosphate group at the 5′ position. Thus modified nucleic acids may include a 2′-O-alkylated sugar more preferred ribose group. The alkyl group is described as R1-R2. R1 is an alkyl with 1 to 20 carbon atoms and R2 is O-alkyl, S-alkyl, NH-alkyl, N-dialkyl, O-aryl, S-aryl, NH-aryl, O-aralkyl, S-aralkyl, NH-aralkyl. Preferred embodiments of 2′-O-alkyl groups are methoxy-, ethoxy-, propyloxy-, isopropyloxy-, methoxy-ethoxy or any other combined alkyl groups.

An additional region of the oligonucleotide or the nucleotide building block may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H. therefore, results in cleavage of the RNA target, thereby greatly enhancing the efficiency of oligonucleotide inhibition of gene expression. Cleavage of the RNA target can be routinely detected by gel electrophoresis and, if necessary, associated nucleic acid hybridization techniques known in the art.

Chimeric antisense conjugates or compounds of the invention may be formed as composite structures of two or more oligonucleotides, modified oligonucleotides, oligonucleosides and/or oligonucleotide mimetics as described above. Such compounds have for example been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

The nucleic acid molecules of the invention may include naturally-occurring or synthetic purine or pyrimidine heterocyclic bases. Purine or pyrimidine heterocyclic bases include, but are not limited to adenine, guanine, cytosine, thymidine, uracil, and inosine. Other representative heterocyclic bases are disclosed in U.S. Pat. No. 3,687,808. The terms “purines” or “pyrimidines” or “bases” are used herein to refer to both naturally-occurring or synthetic purines, pyrimidines or bases.

In an other embodiment the oligonucleotides include non-ionic DNA analogs, such as alkyl- and arylphosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

In yet another embodiment the base units are maintained for hybridization with an appropriate nucleic acid target compound. One such oligomeric compound, an oligonucleotide that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are bound directly or indirectly to azo nitrogen atoms of the amide portion of the backbone. Representative United States patents that teach the preparation of PNA compounds include, but are not limited to, U.S. Pat. Nos. 5,539,082, 5,714,331, and 5,719,262, each of which is herein incorporated by reference. Further teaching of PNA compounds can be found in Nielsen et al. (1994).

In other embodiments the deoxyribose phosphate backbone is replaced with an achiral polyamide backbone. For example thymine-linked aminothioglycol units are prepared. The hybrids of these oligonucleotides have a very high stability compared to underivated oligonucleotides. For more details see Nielsen (1991).

In some embodiments at least one oligonucleotide or nucleotide building block is modified as described in one of the modifications above. The modification can either cover the oligonucleotide or nucleotide building block continuously or irregularly.

In yet another embodiment at least two modifications as described above are combined within one oligonucleotide.

The invention comprises any combination of polymers and linkers, spacer, oligonucleotide, protein, and/or peptide. In preferred embodiments the conjugate or compound comprises or consists of one or more than one oligonucleotide, protein and/or peptide, or one or more oligonucleotides combined with one or more proteins and/or peptides, which are optionally linked via at least one linker and/or spacer. In particular, a conjugate according to the invention comprises or consists of one or more polymers and one or more oligonucleotides, one or more proteins, one or more peptides, one or more linker and/or one or more spacer. The conjugate preferably comprises or consists of an oligonucleotide and a polymer, or an oligonucleotide, a linker and a polymer, or an oligonucleotide and a protein and/or a peptide and a polymer, or an oligonucleotide, a linker, and a protein and/or a peptide and a polymer, or an oligonucleotide, a linker, a spacer, a protein and/or a peptide and a polymer, a protein and/or peptide and a polymer, a protein and/or a peptide, a linker and a polymer, a protein and/or a peptide, a linker, a spacer and a polymer. Such conjugates preferably comprise or consist of one or more oligonucleotides, and/or one or more proteins and/or peptides, and/or one or more linkers, and/or one or more spacer, and one or more polymers, wherein the polymer is preferably a polyalkylen oxide, more preferably PEG. Examples of conjugates are illustrated in the following, which does not represent a limitation of the invention to these examples:

a) polymer-(oligonucleotide, protein or peptide)ₙ
b) (oligonucleotide, protein or peptide)ₙ-polymer
c) polymer-(oligonucleotide, protein or peptide)ₙ-polymer
d) polymer-(polymer, linker and/or spacer-(oligonucleotide, protein or peptide)ₙ)ₓ
e) (linker and/or spacer-(oligonucleotide, protein or peptide)ₙ)ₓ-polymer
f) polymer-(polymer, linker and/or spacer-(oligonucleotide, protein or peptide)ₙ)ₓ-polymer
g) polymer-(polymer, linker and/or spacer-(oligonucleotide, protein or peptide)ₙ)ₓ-linker and/or spacer-polymer
h) polymer-(oligonucleotide)ₙ-(protein and/or peptide)ₘ
i) (oligonucleotide)ₙ-(protein and/or peptide)ₘ-polymer
j) polymer-(oligonucleotide)ₙ-(protein and/or peptide)ₘ-polymer
k) (linker and/or spacer-(oligonucleotide)ₙ-(protein and/or peptide)ₘ)ₓ-polymer
l) (linker and/or spacer-(oligonucleotide)ₙ-(protein and/or peptide)ₘ)ₓ-linker and/or spacer-polymer
m) polymer-(polymer, linker and/or spacer-(oligonucleotide)ₙ-(protein and/or peptide)ₘ)ₓ-linker and/or spacer-polymer
n) polymer-((oligonucleotide)ₙ-linker, spacer and/or polymer-(protein and/or peptide)ₘ)ₓ
o) ((oligonucleotide)ₙ-linker, spacer and/or polymer-(protein and/or peptide)ₘ)ₓ-polymer
p) polymer-((oligonucleotide)ₙ-linker, spacer and/or polymer-(protein and/or peptide)ₘ)ₓ-polymer
q) polymer-(protein and/or peptide)ₘ-(oligonucleotide)ₙ
r) (protein and/or peptide)ₘ-(oligonucleotide)ₙ-polymer
s) polymer-(protein and/or peptide)ₘ-(oligonucleotide)ₙ-polymer
t) polymer-(polymer, linker and/or spacer-(protein and/or peptide)ₘ-(oligonucleotide)ₙ)ₓ
u) (linker and/or spacer-(protein and/or peptide)ₘ-(oligonucleotide)ₙ)ₓ-polymer
v) polymer-(polymer, linker and/or spacer-(protein and/or peptide)ₘ-(oligonucleotide)ₙ)ₓ-polymer
w) polymer-((protein and/or peptide)ₘ-polymer, linker and/or spacer-(oligonucleotide)ₙ)ₓ
x) ((protein and/or peptide)ₘ-polymer, linker and/or spacer-(oligonucleotide)ₙ)ₓ-polymer
y) polymer-((protein and/or peptide)ₘ-polymer, linker and/or spacer-(oligonucleotide)ₙ)ₓ-polymer
z) polymer-linker and/or spacer-((protein and/or peptide)ₘ-polymer, linker and/or spacer-(oligonucleotide)ₙ)ₓ-linker and/or spacer-polymer,
wherein m, n, and x are independent of each other 1 to 20, preferably, 1 to 15, more preferred 1 to 10, even more preferred 1 to 5, and mostly preferred 1 to 3. The term “polymer” in these examples means one or more polymer, which is linked to an end or to any other part of the oligonucleotide, protein and/or peptide.

In yet other embodiments the conjugate or compound is complexed to biological or chemical carriers or is coupled to tissue-type or cell-type directed ligands or antibodies.

In some embodiments of the invention the active modifications of the conjugate or compound comprises oligonucleotides as mentioned herein that have additional nucleotide building blocks. These nucleotide building blocks are chosen in the way, that they support the hybridization with the target region or at least do not influence the hybridization negatively. Those conjugates or compounds with the respective antisense structure of the mRNA of said targets are still within the scope of this invention. The additionally nucleotides in one embodiment are according to the coding region of the mRNA, in yet another embodiment the additional nucleotides are also from the non coding part of the mRNA, including introns and exons. The additionally nucleotide comprises at least one nucleotide, preferably from about 1 to about 10,000 nucleotides, from about 1 to about 5,000 nucleotides, from about 1 to about 3000 nucleotides, from about 1 to about 1,000 nucleotides, from about 1 to about 500 nucleotides, from about 1 to about 100 nucleotides, from about 1 to about 50 nucleotides, from about 1 to about 25 nucleotides, from about 1 to about 10 nucleotides, from about 1 to about 5 nucleotides or from about 1 to about 2 nucleotides bound to at least one of the 3′ and/or 5′ end, in another embodiment on at least one of the 2′ or 5′ end. In yet another embodiment some nucleotide building blocks of those oligonucleotides respectively polynucleotides may be modified or substituted by spacers as described herein.

For more details for derivatisation see for example Gualtieri (2000), R. Schlingensiepen (1997) or Hecht (1997) herein incorporated by reference.

Polymers in the context of this application comprise biocompatible materials, such as polyalkylen oxides, more preferred polyethyleneglycol, for example alpha-, omega-dihydroxylpolyethyleneglycol, biodegradable lactone-based polymers, e.g. polyacrylic acid, polylactide acid (PLA), poly(glycolic acid) (PGA), polypropylene, polystyrene, polyolefin, polyamide, polycyanoacrylate, polyimide, polyethylenterephthalate (PET, PETG), polyethylene terephthalate (PETE), polytetramethylene glycol (PTG), or polyurethane as well as mixtures thereof. Mixture refers to the use of different polymers within the same compound as well as it refers to block copolymers. Block copolymers are polymers wherein at least one section of a polymer is build up from monomers of another polymer.

Selection of such materials depends on a number of factors including the stability and toxicity of the polymer.

In one embodiment an oligonucleotide, a protein and/or a peptide such as an immunostimulator is linked with at least one polymer, more preferred polyalkylen oxide, even more preferred polyethyleneglycol has an average weight of from about 0.05 kg/mol to about 50 kg/mol, in a more preferred embodiment from about 0.05 kg/mol to about 5 kg/mol, or from about 5 kg/mol to about 20 kg/mol or from about 20 kg/mol to about 50 kg/mol. In other embodiments the polymer has a weight of about 0.05 kg/mol to about 1 kg/mol, from about 1 kg/mol to about 10 kg/mol, from about 10 kg/mol to about 25 kg/mol, from about 25 kg/mol to about 50 kg/mol. In yet other embodiments the polymer has a weight of from about 0.05 kg/mol to about 0.15 kg/mol, about 0.15 kg/mol to about 0.25 kg/mol, about 0.25 kg/mol to about 0.35 kg/mol, about 0.35 kg/mol to about 0.45 kg/mol, about 0.45 kg/mol to about 0.55 kg/mol, about 0.55 kg/mol to about 0.65 kg/mol, about 0.65 kg/mol to about 0.75 kg/mol, about 0/5 kg/mol to about 0.85 kg/mol, about 0.85 kg/mol to about 0.95 kg/mol, about 0.95 kg/mol to about 1.05 kg/mol, about 1.05 kg/mol to about 1150 kg/mol, about 1.15 kg/mol to about 1.25 kg/mol, about 1.25 kg/mol to about 1.55 kg/mol, about 1.55 kg/mol to about 1.85 kg/mol, about 1.85 kg/mol to about 2.15 kg/mol, about 2.15 kg/mol to about 2.45 kg/mol, about 2.45 kg/mol to about 2.75 kg/mol, about 2.75 kg/mol to about 3.05 kg/mol, about 3.05 kg/mol to about 3.35 kg/mol, about 3.35 kg/mol to about 3.65 kg/mol, about 3.65 kg/mol to about 4.3 kg/mol, about 4.3 kg/mol to about 4.6 kg/mol, about 4.6 kg/mol to about 5.5 kg/mol, 5.5 kg/mol to about 6.5 kg/mol, 6.5 kg/mol to about 7.5 kg/mol, 7.5 kg/mol to about 8.5 kg/mol, 8.5 kg/mol to about 9.5 kg/mol, 9.5 kg/mol to about 10.5 kg/mol, 10.5 kg/mol to about 11.5 kg/mol, 11.5 kg/mol to about 12.5 kg/mol, 12.5 kg/mol to about 13.5 kg/mol, 13.5 kg/mol to about 14.5 kg/mol, 14.5 kg/mol to about 15.5 kg/mol, 15.5 kg/mol to about 16.5 kg/mol, 16.5 kg/mol to about 17.5 kg/mol, 17.5 kg/mol to about 18.5 kg/mol, 18.5 kg/mol to about 19.5 kg/mol, 19.5 kg/mol to about 20.5 kg/mol, 20.5 kg/mol to about 21.5 kg/mol, 21.5 kg/mol to about 22.5 kg/mol, 22.5 kg/mol to about 23.5 kg/mol, 23.5 kg/mol to about 24.5 kg/mol, 24.5 kg/mol to about 25.5 kg/mol, 25.5 kg/mol to about 26.5 kg/mol, 26.5 kg/mol to about 27.5 kg/mol, 27.5 kg/mol to about 28.5 kg/mol, 28.5 kg/mol to about 29.5 kg/mol, 29.5 kg/mol to about 30.5 kg/mol, 30.5 kg/mol to about 31.5 kg/mol, 30.5 kg/mol to about 31.5 kg/mol, 30.5 kg/mol to about 31.5 kg/mol, 31.5 kg/mol to about 32.5 kg/mol, 32.5 kg/mol to about 33.5 kg/mol, 33.5 kg/mol to about 34.5 kg/mol, 34.5 kg/mol to about 35.5 kg/mol, 35.5 kg/mol to about 36.5 kg/mol, 36.5 kg/mol to about 36.5 kg/mol, 36.5 kg/mol to about 37.5 kg/mol, 37.5 kg/mol to about 38.5 kg/mol, 38.5 kg/mol to about 39.5 kg/mol, 39.5 kg/mol to about 40.5 kg/mol, 40.5 kg/mol to about 41.5 kg/mol, 41.5 kg/mol to about 42.5 kg/mol, 42.5 kg/mol to about 43.5 kg/mol, 43.5 kg/mol to about 44.5 kg/mol, 44.5 kg/mol to about 45.5 kg/mol, 45.5 kg/mol to about 46.5 kg/mol, 46.5 kg/mol to about 46.5 kg/mol, 46.5 kg/mol to about 47.5 kg/mol, 47.5 kg/mol to about 48.5 kg/mol, 48.5 kg/mol to about 49.5 kg/mol, 49.5 kg/mol to about 50.5 kg/mol.

In further preferred embodiments the average molecular weight of the polymer is about 0.1 kg/mol, about 0.2 kg/mol, about 0.3 kg/mol, about 0.4 kg/mol, about 0.5 kg/mol, about 0.6 kg/mol, about 0.7 kg/mol, about 0.8 kg/mol, about 0.9 kg/mol, about 1 kg/mol, about 1.1 kg/mol, about 1.2 kg/mol, about 1.3 kg/mol, about 1.4 kg/mol, about 1.5 kg/mol, about 1.5 kg/mol, about 1.6 kg/mol, about 1.7 kg/mol, about 1.8 kg/mol, about 1.9 kg/mol, about 2 kg/mol, about 2.2 kg/mol, about 2.4 kg/mol, about 2.6 kg/mol, about 2.8 kg/mol, about 3 kg/mol about 3.1 kg/mol, about 3.2 kg/mol, about 3.3 kg/mol, about 3.4 kg/mol, about 3.5 kg/mol, about 3.6 kg/mol, about 3.7 kg/mol, about 3.8 kg/mol, about 3.9 kg/mol, about 4 kg/mol, about 4.1 kg/mol, about 4.2 kg/mol, about 4.3 kg/mol, about 4.4 kg/mol, about 4.5 kg/mol, about 4.6 kg/mol, about 4.7 kg/mol, about 4.8 kg/mol, about 4.9 kg/mol, about 5 kg/mol about 5.5 kg/mol, about 6 kg/mol, about 6.5 kg/mol, about 7 kg/mol, about 7.5 kg/mol, about 8 kg/mol, about 8.5 kg/mol, about 9 kg/mol, about 9.5 kg/mol, about 10 kg/mol, about 11 kg/mol, about 12 kg/mol, about 13 kg/mol, about 14 kg/mol, about 15 kg/mol, about 16 kg/mol, about 17 kg/mol, about 18 kg/mol, about 19 kg/mol, about 20 kg/mol, about 21 kg/mol, about 22 kg/mol, about 23 kg/mol, about 24 kg/mol, about 25 kg/mol, about 26 kg/mol, about 27 kg/mol, about 28 kg/mol, about 29 kg/mol, about 30 kg/mol, about 31 kg/mol, about 32 kg/mol, about 33 kg/mol, about 34 kg/mol, about 35 kg/mol, about 36 kg/mol, about 37 kg/mol, about 38 kg/mol, about 39 kg/mol, about 40 kg/mol, about 41 kg/mol, about 42 kg/mol, about 43 kg/mol, about 44 kg/mol, about 45 kg/mol, about 46 kg/mol, about 47 kg/mol, about 48 kg/mol, about 49 kg/mol, about 50 kg/mol.

The term about refers to the fact, that commercially available polymers are not completely homogenous in weight, but are fractions of polymers with about a certain weight not defined accurately by the mass, but differs around this mentioned average mass/weight dependent of the absolute mass and according to the way of synthesis and/or purification. The average molecular weight refers to the weight-average molecular weight. In one embodiment one of two polymers is linked to the 3′-end and the second polymer is linked to the 5′-end or the respective portion of the nucleotide building block of the oligonucleotide.

In a further preferred embodiment the polymer linked to the 3′-end has a higher weight compared with the polymer linked to the 5′-end. In a further preferred embodiment the 3′-polymer has the 1.5 to 100-fold molecular weight of the polymer linked to the 5′-end.

In another preferred embodiment the polymer linked to the 5′-end has a higher molecular weight compared with the polymer linked to the 3′-end. In a further preferred embodiment the 3′-polymer has the 1.5- to 100-fold weight of the polymer linked to the 5′-end.

In further preferred embodiments the weight ratio of the polymers is 1:1.5, 1:2, 1:2.5, 1:3, 1:3.5, 1:4, 1:4.5, 1:5, 1:5.5, 1:6, 1:7, 1:7.5, 1:8, 1:8.5, 1:9, 1:9.5, 1:10, 1:11, 1:12, 1:13, 1:14, 1:15, 1:16, 1:17, 1:18, 1:19, 1:20, 1:22, 1:24, 1:26, 1:28, 1:30, 1:32, 1:34, 1:36, 1:38, 1:40, 1:42, 1:44, 1:46, 1:48, 1:50, 1:55, 1:60, 1:65, 1:70, 1:75, 1:80, 1:85, 1:90, 1:95, 1:100.

In further preferred embodiments the smaller polymer has an average molecular weight of 0.4 kg/mol, 0.6 kg/mol, or 0.8 kg/mol and the other polymer has an average weight of 0.8 kg/mol, 1 kg/mol, 1.2 kg/mol, 1.4 kg/mol, 1.6 kg/mol, 1.8 kg/mol, 2 kg/mol, 3 kg/mol, 4 kg/mol or 5 kg/mol.

In even more preferred embodiments the molecular weight of the polymer is between 200 Da to 50 000 Da, preferably 400 Da to 40 000 Da, more preferred 400 Da to 20 000 Da, and even more preferred 400 Da to 10 000 Da. In particular, the molecular weight of the polymer is 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 5500, 6000, 6500, 7000, 7500, 8000, 8500, 9000, 9500, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 460000, 47000, 48000, 49000, 50000, 75000, or 1000000 Da.

Preferably, the oligonucleotide, the protein and/or the peptide is linked with at least two polymers, which are identical or different in the molecular weight of the polymers. Even more preferably, a conjugate or compound of the invention comprises a polymer for example polyalkylen oxide such as PEG, and an oligonucleotide, wherein at least one PEG is linked to the 5′-end of the oligonucleotide and at least one PEG is linked to the 3′-end of the oligonucleotide, wherein the molecular weight of the PEG linked to the 5′- and 3′-end of the oligonucleotide is identical and is <5000 Da, or wherein the molecular weight of the PEG linked to the 5′- and 3′-end of the oligonucleotide is different. When the molecular weight of the PEG linked to the 5′- and 3′-end of the oligonucleotide is identical, the molecular weight of the PEG is preferably 200, 300, 400, 500, 600, 700, 800, 900, 1000, 1250, 1500, 1750, 2000, 2250, 2500, 2750, 3000, 3250, 3500, 3750, 4000, 4250, 4500 or 4750 Da. When the molecular weight of the PEG linked to the 5′- and 3′-end of the oligonucleotide is different, the molecular weight of the polyethylene glycol linked to the 5′-end is preferably 200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000 Da and the molecular weight of the polyethylene glycol linked to the 3′-end of the oligonucleotide is preferably 200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000 Da.

In case of double stranded RNA or DNA, one end of one strand is linked to a polymer, both ends of one strand or both ends of both strands are linked to a polymer, or both ends of one strand are linked to a polymer and one end of the other strand is linked to a polymer.

Moreover, one or more polymers are linked to the 5′- and 3′-end of an oligonucleotide or to the N- and the C-terminus of a protein or peptide. Alternatively, one or more polymers are linked to one end of the oligonucleotide, the protein and/or the peptide, and any other possible linking point in the oligonucleotide, protein and/or peptide. In a further alternative, one or more polymers are linked to both ends of the oligonucleotide, protein and/or peptide, and any other linking point in the oligonucleotide, protein and/or peptide. Preferably, the oligonucleotide, the protein and/or the peptide is specifically linked with one or more polymers at selected positions of the oligonucleotide, the protein, and/or the peptide.

In preferred embodiments, a polymer is linked to each end of the oligonucleotide, protein and/or peptide, wherein the polymers are identical in size having for example a molecular weight of 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 5500, 6000, 6500, 7000, 7500, 8000, 8500, 9000, 9500, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 460000, 47000, 48000, 49000, 50000, 75000 or 100000 Da. As a specific type of polymers might slightly differ in size depending on the isolating, synthetization, degradation over the time, the molecular weights indicated represent an average molecular weight. Moreover, the term “identical” comprises differences in the molecular weight of the polymer of 1 to 50%, preferably 1 to 30%, more preferred 1 to 20%, and even more preferred 1 to 10%

Polymers of identical molecular weight linked with the 5′-end and the 3′-end of an oligonucleotide, and/or the N-terminus and the C-terminus of a protein and/or a peptide have preferably a molecular weight of 200, 300, 400, 500, 600, 700, 800, 900, 1000, 1250, 1500, 1750, 2000, 2250, 2500, 2750, 3000, 3250, 3500, 3750, 4000, 4250, 4500 or 4750 Da.

In even more preferred embodiments, a polymer is linked to each end of the oligonucleotide, protein and/or peptide, wherein the polymers differ in size. Preferred combinations of molecular weight of polymers linked with an oligonucleotide, protein and/or peptide are for example polymers of 200 or 300, 400 or 500, 600 or 700, 800 or 900, 1000 or 1250, 1500 or 1750, 2000 or 2250, 2500 or 2750, 3000 or 3250, 3500 or 3750, 4000 or 4250, 4500 or 4750 or 5000 and 1000 or 1250 or 1500 or 1750 or 2000 or 2250 or 2500 or 2750 or 3000 or 3250, 3500 or 3750 or 4000 or 4250 or 4500 or 4750 or 5000 or 5500 or 6000 or 6500 or 7000 or 7500 or 8000 or 8500 or 9000 or 9500 or 10000 or 11000 or 12000 or 13000 or 14000 or 15000 or 16000 or 17000 or 18000 or 19000 or 20000 or 21000 or 22000 or 23000 or 24000 or 25000 or 26000 or 27000 or 28000 or 29000 or 30000 or 31000 or 32000 or 33000 or 34000 or 35000 or 36000 or 37000 or 38000 or 39000 or 40000 or 41000 or 42000 or 43000 or 44000 or 45000 or 460000 or 47000 or 48000 or 49000 or 50000 or 75000 or 100000 Da. Preferably, molecular weight combinations of polymers linked with an oligonucleotide, protein and/or peptide are polymers of 400 Da and 800 Da, 400 Da and 2000 Da, 400 Da and 5000 Da, or 400 Da and 20000 Da.

In one embodiment the at least one polymer of the conjugate or compound is linear (single stranded). In yet another embodiment the polymer is branched. There can be one branch or several branches in the polymer, the polymer in one embodiment is polyoxyalkylene, more preferred polyethyleneglycol. Preferably, at least two, for example 2, 3, 4, 5, 6, 7, 8, 9, or 10 polymers are connected with each other via a linker, e.g., a Lys core leading to a branched polymer.

The linkage between the oligonucleotide, the protein and/or the peptide and the at least one polymer is optionally by at least one linker. In some embodiments linkers are sensitive to enzymes, pH value and/or other parameters, which allow the oligonucleotide to be split of the polymer under specific conditions. Linkers are known in the art. For more details see also Hermanson (1996). Examples for homobifunctional linkers are Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB, such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), N,N′-hexamethylene-bis(iodoacetamide).

The linkage with heterobifunctional linkers is preferred since the linkage can be better controlled. Examples for heterobifunctional linkers are amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteypaminobenzoate (sIAB), sulfosuccinimidyl(4-iodoacetyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB). N-(γ-maleimidobutyryloxy)succinimide ester (GMBs). N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino)hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA). N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH). N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumarin-3-acetate (sulfo-sAMCA), ρ-nitrophenyl diazopyruvate (ρNPDP), ρ-nitrophenyl-2-diazo-3,3,3-trifluoropropionate (PNP-DTP), sulfhydryl-reactive and photoreactive cross-linkers such as1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In a preferred embodiment the polyethylene oxide is linked with the oligonucleotide using carbodiimide coupling. For more details see Hermanson (1996).

### Synthesis

The synthesis and purification, respectively, of an oligonucleotide, a protein and/or a peptide, e.g., an immunostimulator, are well known to those skilled in the art. For more details see for example Gualtieri (2000), R. Schlingensiepen (1997) or Hecht (1997) herein incorporated by reference.

For use in the instant invention, the nucleic acids or amino acids are for example synthesized de novo using any of a number of procedures well known in the art. Such compounds are referred to as synthetic nucleic acids, for example, the cyanoethyl phosphoramidite method (Beaucage, S. L., and Caruthers, M. H., Tet. Let. 22:1859, 1981); or nucleoside H-phosphonate method (Garegg et al., Tet. Let. 27:4051-4054, 1986; Froehler et al., Nucl. Acid. Res. 14: 5399-5407, 1986, Garegg et al, Tet. Let. 27:4055-4058, 1986, Gaffney et al., Tet. Let. 29: 2619-2622, 1988). These chemistries are for example performed by a variety of automated oligonucleotide synthesizers available on the market.

Alternatively, nucleic acids or amino acids, i.e., oligonucleotides or proteins or peptides are for example produced on a large scale in plasmids, (see, e.g., Sambrook, et al., Molecular Cloning: A Laboratory Manual, Cold Spring Harbor Laboratory Press, New York, 1989) and are for example separable into smaller pieces or administered in the whole. Nucleic acids are for example prepared from existing nucleic acid sequences (e.g. genomic or cDNA) and amino acids are for example prepared from existing amino acid sequences using known techniques, such as those employing restriction enzymes, exonucleases or endonucleases. Nucleic acids and amino acids prepared in this manner are referred to as isolated nucleic acids and isolated amino acids, respectively. The term oligonucleotide encompasses both synthetic and isolated nucleic acids. The term protein or peptide encompasses both synthetic and isolated amino acids.

One preferred embodiment for the synthesis of oligonucleotide-conjugates is solid phase synthesis using a first nucleotide coupled to a solid support, e.g. controlled pore glass (CPG) and nucleotide phosphoroamidites. The 5′-hydroxy group of the nucleotide phosphoroamidites is protected by dimethoxytrityl (DMT) and the exocyclic nitrogen atoms of the base are protected e.g. with benzoyl preferable for adenosine and cytidine, and isobutyryl preferable for guanosine. A synthesis cycle is done as follows:

The DMT protecting group of the terminal 5′-hydroxy group of the support-bound nucleotide chain is removed and the hydroxy function is reacted with the phosphoramidite group of the added nucleotide resulting in a phosphite triester linkage. This is stabilized by oxidation to a phosphate linkage. In a preferred embodiment this oxidation is done by a sulfurizing reagent, e.g., 3H-1,2-benzodithiol-3-one, 1,1-dioxide (Beaucage reagent) resulting in thiophosphate linkage. Non-reacted 5-hydroxy groups are capped to prevent synthesis of failure sequences. This procedure is repeated until the desired number of nucleotides is added.

In one embodiment the polymer is linked to the 5′-terminus of the oligonucleotide by using a DMT-protected phosphoramidite-derivative of the polymer as educt in the last synthesis cycle. Cleaving from the support results in deprotection of the bases and gives the 5′-DMT-protected oligonucleotide or conjugate respectively. This DMT-on product preferably is purified before detrylation.

Proteins are preferably synthesized by liquid-phase, in particular with regard to large-scale production, or more preferably by solid-phase synthesis. Solid-phase synthesis (SPPS), for example according to Merrifield, allows the synthesis of natural or even non-natural proteins or peptides, which are difficult or even non-expressable in bacteria, the incorporation of unnatural amino acids, peptide/protein backbone modification, and the synthesis of D-proteins consisting of D-amino acids. Small solid beads (e.g., polystyrene resin or polyamide resin), insoluble yet porous, are treated with functional units (“linker”) on which a protein or peptide chain is buildable. The protein or peptide remains covalently attached to the bead until cleaved from it by a reagent such as trifluoroacetic acid. The protein or peptide is “immobilized” on the solid-phase and is retained for example during a filtration process. The general principle of SPPS is one of repeated cycles of coupling and deprotection. The free N-terminal amine of a solid-phase attached protein or peptide is coupled to a single N-protected amino acid unit. This is then deprotected, revealing a new N-terminal amine to which a further amino acid is attachable. The important consideration in each step is to generate high yield in each step, whereby each amino acid is added in each step in major excess (e.g., 2 to 10×). Solid-phase protein/peptide synthesis proceeds in a C-terminal to N-terminal fashion. For coupling the amino acids, the carboxyl group is preferably activated for example by carbodiimides such as dicyclohexylcarbodiimide (DCC) or diisopropylcarbodiimide (DIC), or by aromatic oximes such as 1-hydroxy-benzotriazole (HOBt), 1-hydroxy-7-aza-benzotriazole (HOAt), HBTU, HATU, or PyBOP. The N-termini of amino acid monomers is protected for example by Fmoc (9-fluorenylmethyl carbamate) or t-Boc (tert-butyl oxy carbonyl), and added onto a deprotected amino acid chain. To remove t-Boc from a growing protein or peptide chain, acidic conditions are used (for example neat trifluoroacetic acid (TFA)). Removal of side-chain protecting groups and cleavage of the protein or peptide from the resin at the end of the synthesis is achieved for example by incubating in hydrofluoric acid. To remove Fmoc from a growing peptide chain, basic conditions (for example piperidine in DMF) are used. Removal of side-chain protecting groups and cleavage of the protein or peptide from the resin is achieved for example by incubating with TFA, deionized water, and triisopropylsilane. A further protective group is for example a benzyloxy-carbonyl (Z) group, which is preferably used for side chain protection, and which is for example removed by HBr/acetic acid or hydrogenation. Another protective group is an alloc protecting group, which is preferably used to protect a carboxylic acid, a hydroxyl or an amino group when an orthogonal deprotection scheme is required. The alloc protecting group is preferably removed using for example tetrakis(triphenylphosphine)-palladium along with a mixture of chloroform, acetic acid, and N-methylmorpholine (e.g., 37:2:1). Also cyclic proteins or peptides are synthesizable via a solid phase synthesis. Preferably, side chains are protected by different protecting groups, which allow specific deprotection of certain side chains depending on the reaction conditions such as pH. The following amino acids are preferably targeted for modification: Glu, Asp, Cys, Lys, Arg, Ser, Tyr, and His, i.e., for example the alcohol functions of Ser and Thr, the phenol of Tyr, the guanidine of arginine, or the thiol of Cys.

Preferably, the NH₂ groups of the protein or peptide are selectively protected for a specific coupling of a polymer for example PEG. For example a protecting group A is protecting the N-terminus of the protein or peptide, a protecting group B is protecting assigned Lys-side chain NH groups, and the protecting group C is connected to all NH₂ groups of side chains, which shall not be linked to the polymer. For example the protective groups A, B, and C are used for the synthesis of the protein or peptide and the linkage of a polymer, group A is selectively deprotected in a first step and the protein or peptide is selectively linked to a polymer at site X. In a second step protective group B is selectively removed and the protein or peptide is selectively linked to further PEG at the site Y. In a last step the protein or peptide is cleaved from the support and the remaining protecting groups C, which are not intended to be linked to a polymer, are removed.

Furthermore, the protein or peptide synthesis is based on microwave technique, wherein each single step is done in a monomode microwave apparatus for example within 30 sec.

Alternatively, larger proteins or peptide are constructed via native chemical ligation of two or more smaller peptides or proteins. In native chemical ligation a peptide or protein containing a C-terminal thioester reacts with another protein or peptide containing an N-terminal cysteine for example in the presence of an exogenous thiol catalyst. In a first step a transthioesterfication occurs. In a second step the product preferably rearranges irreversibly under the usual reaction conditions to form an amide bond. Protein- or peptide-thioesters usable in native chemical ligation are for example prepared by BOC chemistry SPPS. Moreover, protein or polypeptide C-terminal thioesters produced by recombinant DNA techniques react with a N-terminal Cys containing protein or polypeptide by native ligation chemistry to provide large semisynthesized proteins or polypeptides.

In yet another embodiment at least one polymer is linked to the oligonucleotide, protein and/or peptide in solution. The linkage of at least one polymer with the oligonucleotide, protein and/or peptide is done by activating certain groups of the oligonucleotide, protein and/or peptide and/or the polymer and then linking those molecules. Preferred for coupling of the polymer are the 3′ and/or the 5′ end of the oligonucleotide or the N-terminus and/or the C-terminus of the protein and/or peptide. In an alternative embodiment, conjugates comprising or consisting of an oligonucleotide and a protein and/or peptide, one or more polymers are preferably linked to the 5′ end of the oligonucleotide and one or more polymers are linked to the C-terminus of the protein and/or peptide; or one or more polymers are preferably linked to the N-terminus of the protein and/or the peptide and one or more polymers are linked to the 3′ end of the oligonucleotide; or one or more polymers are preferably linked to the 5′ end of the oligonucleotide and one or more polymers are linked to the N-terminus of the protein and/or peptide; or one or more polymers are preferably linked to the 3′ end of the oligonucleotide and one or more polymers are linked to the C-terminus of the, wherein the polymer is in particular a polyalkylen oxide, more preferably PEG. Preferably the polymers are directly linked to the oligonucleotide and/or peptide or more preferably via one or more linkers and/or one or more spacers.

In yet other embodiments at least one polymer is coupled to another part of the oligonucleotide, protein and/or peptide. In some embodiments this part of the oligonucleotide is the phosphate-group, in other embodiments one or more polymers are coupled to the 2′-O of the sugar moiety of the nucleotide or the respective moiety of the nucleotide building block. In yet other embodiments the polymer is coupled to an activated group of the base of the nucleotide. In another embodiment instead of at least one nucleotide or one amino acid a spacer is used. Preferably, the spacer is used in combination with a linker, or substitutes the linker. The at least one polymer can be coupled to this spacer as well. Several polymers can be coupled to the oligonucleotide at different sites of the above described parts of the oligonucleotide.

In other embodiments one or more polymers are coupled to any aminoacid of the protein or peptide, in particular to SH-, OH-, or NH-groups of one or more amino acids. Proteins and peptides, respectively, couple preferably through their N-terminals (alpha-amine) and lysine side chain (epsilon-amine) functional groups. In addition, a free cysteine residue in a protein or peptide is PEGylated, or in the absence of free cysteine such cysteine residues are introducible into a protein or peptide by genetic engineering to create a specific site for PEGylation with site-directed mutagenesis or by modifications of amino groups with Iminothiolane. In preferred embodiments referring to conjugates comprising an oligonucleotide, a protein and/or a peptide, one or more polymers are preferably linked to one or both ends of the oligonucleotide, the protein and/or the peptide; or one or more polymers are linked to one or both ends of the oligonucleotide, the protein and/or the peptide, and/or to another part of the oligonucleotide such as the phosphate-group and/or the 2′-O of the sugar moiety of the oligonucleotide, and/or to another part of the protein and/or peptide such as SH— or NH-groups of one or more amino acids. The polymer is preferably linked to the oligonucleotide, protein and/or peptide via a linker and/or a spacer, wherein one or more polymers are linked to the end and/or another part of the linker and/or the spacer and/or the oligonucleotide, protein and/or peptide is linked to the end and/or another part of the linker and/or the spacer.

Optionally, a linker and/or a spacer is connected to the base, sugar, phosphate moiety, or any of its derivatives of an oligonucleotide, wherein the linker and/or spacer carries a functional group suitable for reaction with a polymer, e.g., a polyalkylen oxide such as PEG. Preferably, the functional group is protected by a protection group, which is removed before the reaction with the polymer. Same is applicable for a protein or peptide, wherein a linker and/or spacer is combinable with any amino acid or a sugar moiety of a glycoprotein.

Polymers are synthesized by polymerisation of the respective monomers, which are synthesized or isolated from natural sources. Most of them are commercially available or are achieved by standard synthesis, see for example Braun (2005).

Covalent attachment of a polymer, more preferred polyoxyalkylene, even more preferred polyethyleneglycol to an oligonucleotide, a protein and/or a peptide is accomplished by known chemical synthesis techniques. For more details see Hermanson (1996).

Linking the oligonucleotide, the protein and/or the peptide, e.g., an immunostimulator with at least one or at least two polymers is achieved by the reactivity of the active groups of those molecules such as COOH, CHO, OH, NH₂, SH etc. In general one functional group is activated and then linked with another active group of an oligonucleotide, a protein and/or a peptide.

In some preferred embodiments the selective derivatisation of preferred functional groups is achieved by protecting other functional groups that should not be linked with protecting groups. After linkage of the oligonucleotide, the protein and/or the peptide for example an immunostimulator with the at least one polymer those protection groups are removed.

In some embodiments the linkage of the oligonucleotide, the protein and/or the peptide such as an immunostimulator with the at least on polymer is on a solid phase, in yet other embodiments the linkage is done in solution.

A preferred method for the production of a conjugate or a compound of the present invention comprises the following steps: a) isolating or synthesizing the oligonucleotide, the protein or the peptide, including modified oligonucleotide, proteins, or peptides, b) protecting the 5′-end or the 3′-end of the oligonucleotide, and/or the N-terminus or C-terminus of the protein or peptide and/or other reactive groups of the amino acids forming the protein or peptide, and/or a phosphate group, a sugar moiety, and/or a base of the oligonucleotide, and/or an SH—, OH- and/or a NH-group or any other functional group of a protein or a peptide such as tyrosin-OH or a cycloprotein or -peptide and/or a sugar moiety of a glycoprotein, and c) linking at least one polymer for example polyalkylen oxide, e.g., PEG, with the unprotected 3′-end or 5′-end of the oligonucleotide and/or the unprotected C- or N-terminus of a protein or peptide. Optionally the method further comprises the steps: d) deprotecting the protected 5′-end or 3′-end of the oligonucleotide, and/or the N-terminus or C-terminus of the protein or peptide, and/or a phosphate group, a sugar moiety, and/or a base of the oligonucleotide, and/or an SH- and/or a NH-group of a protein or a peptide, and e) linking at least one polymer for example polyalkylen oxide such as PEG with the deprotected 5′-end or 3′-end of the oligonucleotide, and/or the deprotected phosphate group, sugar moiety, and/or base of the oligonucleotide and/or the deprotected N- or C-terminus of the protein or peptide and/or the deprotected SH- and/or a NH-group of the protein or peptide. Once at least one polymer for example polyalkylen oxide such as PEG is connected to the oligonucleotide, the protein and/or the peptide, the conjugate is optionally purified via one or more steps of purification, e.g., FPLC such as ion exchange FPLC, reverse phase HPLC, ultrafiltration, etc. In general, preferably the oligonucleotide, protein and/or peptide is conjugated with a polymer such as polyalkylen oxide, e.g., PEG, then the conjugate is purified for example via FPLC or HPLC, dried for example in a SpeedVac, desalted for example via ultrafiltration and dried again. In case of conjugation of a further polymer such as polyalkylen oxide, e.g., PEG, the sample is dissolved in water or buffer, incubated with a polymer, purified, dried, desalted, and dried again.

The conjugate is preferably stored in dried form or dissolved in water or buffer such as TEAA, or TEA.

### 5′-PEGylation of Oligonucleotides

In one preferred embodiment for 5′-polymer linkage, more preferred linking a polymer to an oligonucleotide (e.g. PEGylation), the polymer (e.g., PEG) is activated as phosphoramidite. In this embodiment DMT-protected-PEG is prepared by reacting an excess of the polymer with dimethoxytriphenylmethylchloride in the presence of for example triethanolamine (TEA) and 4-dimethylaminopyridine (DMAP). Reaction of DMT-PEG with 2-cyanoethyl-diisopropylchlorophosphoramidite in the presence of diisopropylethylamine gives access to DMT-polymer-phosphoramidites which than is preferably used as educt in the final cycle of standard oligonucleotide synthesis on CPG-solid-support.

In one embodiment intermediates are purified by column chromatography. The conjugate is preferably purified by for example RP-HPLC as DMT-on product. After detritylation with acetic acid, the final product is preferably desalted by ultrafiltration.

### 5′-Polymer Linkage of Oligonucleotides

In another preferred embodiment an oligonucleotide is linked with at least one polymer (e.g. polyoxyethylene, PEG) at the 5′-end. The oligonucleotide is prepared by conventional oligonucleotide synthesis, using for example a phosphoramidite in the final cycle, for example resulting in a 5′-amino group (5′-amino-modifier) or a 5′-thiol group (5′ thiol-modifier). A 5′-amino-modifier respectively a 5′-thiol-modifier is a linker covalently binding to the 5′-hydroxy-group of the sugar moiety of the nucleotide building block and adds a NH₂ respectively an SH group as active group for linking with the polymer. This group of the modifier is linked to a commercially available or custom synthesized functionalized PEG after deprotection according to standard procedures as described for example by Hermanson (1996).

In one preferred embodiment where the functional group of the oligonucleotide is NH₂ suitable functionalized polymers (e.g. PEG) are for example NHS-ester. NHS-carbamates or NHS-carbonates, 4-nitrophenylester or otherwise activated carboxylic acids.

In yet other embodiments, where the functional group of the oligonucleotide, e.g., an immunostimulator, is the SH-group, the functional groups of the polymer, more preferred the PEG, are for example maleimides or dithiodipyridyl-activated sulfhydryls.

### 3′-PEGylation of Oligonucleotides

In an alternative preferred embodiment for linking a polymer to the 3′-end of an oligonucleotide (e.g. 3′-PEGylation), the polymer is linked to a carboxy-functionalized CPG-support. This in one embodiment is prepared by succinylation of aminopropylated CPG-support using succinic anhydride in the presence of DMAP. The carboxyl groups of the succinylated CPG are activated by for example O-benzotriazole-N,N,N′,N′-tetramethyl-uronium-hexafluoro-phosphate (HBTU) in the presence of for example DMAP and diisopropylethylamine and subsequently reacted with the polymer (e.g., PEG). The resulting polymer-modified support is used as solid support in standard oligonucleotide synthesis, resulting in an oligonucleotide linked to the polymer via its 3′-terminus. Cleavage from the support using for example 32% NH₃ results in the deprotected, PEG-modified DMT-on product which then is detritylated, e.g., with acetic acid.

### 3′-PEGylation of Oligonucleotides

In yet another preferred embodiment a 3′-modified oligonucleotide is prepared by standard solid phase oligonucleotide chemistry using commercially available or custom synthesized supports, for example a 3′-amino-modifier or a 3′-thiol-modifier. A 3′-amino-modifier respectively a 3′-thiol-modifier in this context is a solid support, where the 3′ terminus of a nucleotide is linked to the solid support by a liner that results in a 3′ terminal NH₂ or SH function after cleavage of the oligonucleotide from the column. In one embodiment the resulting 3′-modified oligonucleotide is linked to a commercially available or custom synthesized functionalized PEG according to standard procedures as described for example by Hermanson (1996).

In further preferred embodiments the functional groups for linking a polymer (e.g. PEG) to aminomodified oligonucleotides are for example NHS-ester. NHS-carbamates or NHS-carbonates, 4-nitrophenylester or otherwise activated carboxylic acids. Functional groups suitable for polymer linkage (PEG conjugation) to sulfhydryls are maleimide or dithiodipyridyl-activated sulfhydryls.

3′,5′-di-polymer Linkage of Oligonucleotides

In yet an even more preferred embodiment the polymer (e.g., PEG) is linked to the 3′- and 5′-end of the oligonucleotide. In one embodiment these oligonucleotides linked with two polymers are prepared by combining above described embodiments using for example a DMT-polymer-phosphoramidite in the final cycle of the oligonucleotide synthesis on a polymer-modified CPG-support.

3′,5′-di-PEGylation of Oligonucleotides

In yet another more preferred embodiment compounds comprising at least one polymer (e.g., PEG) linked to both ends, the 3′- and the 5′-end, of the oligonucleotide are prepared by combining embodiments of solution phase chemistry as described above using a 3′-modifier and a 5′-modifier. In yet another embodiment the linkage of an oligonucleotide with at least two polymers is done by a combination of solid phase chemistry and solution phase chemistry.

In yet another embodiment the oligonucleotide, e.g., an immunostimulator, has two identical functional groups (e.g. SH, OH, NH₂). This oligonucleotide is suitable to conjugate identical polymers.

In yet another more preferred embodiment the oligonucleotide, for example an immunostimulator, has at least two different functional groups (e.g. SH, OH, NH₂). This oligonucleotide is suitable to conjugate different polymers. Preferred polymer combinations comprise for example PEG and polyacrylic acid, polylactic acid, poly(glycolic acid), polypropylene, polystyrene, polyolefin, polyamide, polycyanoacrylate, polyimide, polyethylenterephthalate (PET. PETG), polyethylene terephthalate (PETE), polytetramethylene glycol (PTG), or polyurethane as well as mixtures thereof.

### PEGylation of Proteins or Peptides

Preferably the protein or peptide is synthesized according to Merrifield, wherein side chains of the amino acids as well as the N-terminal end of the protein or peptide.

In a preferred embodiment of protein PEGylation, PEG is for example activated with trichloro-s-triazine (TsT; cyanuric acid). Reaction of TsT with PEG results for example in the formation of an activated derivative with an ether bond to the hydroxyl group of the polymer. TsT-activated PEG leads for example to linkage to sulfhydryl and amine groups of the protein or the peptide, or to the linkage to the phenolate and phenyl ring, respectively, of tyrosine.

In an alternative embodiment. PEG is activated in a first step preferably with an anhydride such as succinic anhydride or glutaric anhydride, resulting in PEG having carboxylates at one or both ends; alternatively, the terminal hydroxyl group of PEG is treated with phosgene or oxidized to carboxylate. Subsequently, the carboxylate groups are activated NHS esters for example reacting with sulfhydryl and/or hydroxyl groups as well as with primary and secondary amines. NHS ester groups react preferably with the alpha-amines at the N-terminus of the protein or peptide. In a preferred alternative. PEG is activated either with N-hydroxysuccinimidyl chloroformate or N,N′-disuccinimidyl carbonate. In case of a protein also further amine binding sites may be available.

In a further embodiment, acylation of PEG with succinic anhydride or glutaric anhydride gives bis-modified products having carboxylates at both ends. Modification of mPEG yields the monosubstituted derivative containing a single carboxylate. Once the carboxylate-PEG modification is formed, it is suitable to be coupled to amine-containing molecules using a carbodiimide activation for example.

In another embodiment, PEG is activated with N,N′-carbonyldiimidazole (CDI) and couples subsequently to a protein or peptide. Such CDI-activated PEG is stable for years in a dried state or in organic solvents devoid of water.

Additional alternatives for the activation and coupling of PEG to a protein or peptide are the reaction of PEG with epichlorhydrin under alkaline conditions leading to an epoxy derivative of PEG, or creating a sulfhydryl-reactive PEG via an active ester-maleimide heterobifunctional cross-linker, or a N-maleimido-6-aminocaproyl ester of 1-hydroxy-2-nitro-4-benzene sulfonic acid, or the Moffatt oxidation.

The newly synthesized protein or peptide comprises protecting groups, which are removeable under selective conditions for example acidic or basic conditions, or hydrogenation. After selective deprotection of assigned coupling sites the solid-support bound the protein or peptide is incubated with a polymer, an activated polymer for example an activated polyalkylen oxide such as PEG, which binds to the reactive groups of the protein or peptide. Afterwards the protein or peptide is cleaved from the support and the deprotected in particular selectively deprotected protein or peptide for example PEGylated at an amino acid side chain is optionally incubated another time with a polymer such as PEG, preferably an activated PEG. The second incubation results for example in a further PEGylation at another coupling site e.g. at the N-terminal end of the protein or peptide. Alternatively, the N-terminal protective group of a solid support bound, site chain protected protein or peptide is removed under specific conditions for example acidic or basic conditions, the protein or peptide is incubated with a polymer preferable an activated polymer leading to a protein or peptide linked to a polymer at the N-terminal end. The protein or peptide is cleaved from the support and optionally incubated a second time with a polymer to link the polymer to the C-terminal end, wherein the C-terminal is preferably activated. Afterwards the protective groups of the side chains are removed. Alternatively, the protective groups of the side chains are removed, after a polymer is linked to the N-terminal end, and in a final step the protein or peptide is cleaved from the support. In another alternative, the protein or peptide having a protected N-terminal end and side chains is cleaved from the support and incubated with a polymer preferably with an activated polymer. Once the polymer is linked to the C-terminal end, the protective groups of the side chains and the N-terminal end are optionally specifically removed for example under acidic and/or basic conditions and the protein is optionally incubated a further time with a polymer. In a further alternative, a polymer is linked to the sugar moiety of a glycoprotein and optionally to the N-terminal end, the C-terminal end and/or a side chain of the protein. Preferably, a sugar moiety of a glycoprotein is specifically modifiable by oxidation using sodium periodate to generate reactive aldehyde functions. These functional groups are suitable to be directly conjugated or used for coupling via heterobifunctional linkers for example PDPH or MPBH, both leading to SH-reactive functional groups. The sulfhydryl group finally reacts either directly with a polymer or via a linker and/or spacer with a polymer.

### Application

Besides being useful in human treatment, the present invention is also useful for other subjects including for example veterinary animals, reptiles, birds, exotic animals and farm animals, including for example mammals, rodents, and the like. Mammals include horses, dogs, pigs, cats, or primates (for example, a monkey, a chimpanzee, or a lemur). Rodents include rats, rabbits, mice, squirrels, or guinea pigs.

The oligonucleotide, protein and/or peptide linked with at least one polymer according to this invention is suitable to be administered by different routes. These routes of administration include, but are not limited to, electroporation, epidermal, impression into skin, intra-arterial, intra-articular, intra-cranial, intra-thecal, intra-cerebral, intra-dermal, intra-lesional, intra-muscular, intra-nasal, intra-ocular, intra-peritoneal, intra-prostatic, intra-pulmonary, intra-spinal, intra-tracheal, intra-tumoral, intra-venous, intra-vesicle placement within cavities of the body, nasal inhalation, oral, pulmonary inhalation (e.g., by inhalation or insufflation of powders or aerosols, including by nebulizer), subcutaneous, subdermal, transdermal, or topical (including ophthalmic and to mucous membranes including vaginal and rectal delivery). Topical administration further comprises administration of the skin, the eyes and the ears.

Dosing is dependent on severity and responsiveness of the disease state to be treated, with the course of treatment lasting from several days to several months, or until a cure is effected or a diminution of the disease state is achieved. Optimal dosing schedules are for example calculated from measurements of drug accumulation in the body of the patient. Persons of ordinary skill easily determine optimum dosages, dosing methodologies and repetition rates. Optimum dosages may vary depending on the relative potency of individual oligonucleotides with at least one polymer, and are generally be estimated based on EC₅₀ values found to be effective in in vitro and in vivo animal models. In general, dosage is for example from 0.01 μg to 100 g per kg of body weight, and may be given once or more, e.g., two, three, four, five, six, seven, eight, nine or ten times daily, weekly, monthly or yearly, or even once every 2 to 20 years. Persons of ordinary skill in the art easily estimate repetition rates for dosing for example based on measured residence times and concentrations of the drug in body fluids or tissues. Following successful treatment, it may be desirable to have the patient undergo maintenance therapy to prevent the recurrence of the disease state, wherein the oligonucleotide, protein and/or peptide with at least one polymer is administered in maintenance doses, ranging from 0.01 μg to 100 g per kg of body weight, once or more, e.g., two, three, four, five, six, seven, eight, nine or ten times daily, weekly, monthly or yearly, or even once every 2 to 20 years.

In one embodiment the at least one polymer linked with the oligonucleotide, protein and/or peptide is in a pharmaceutically acceptable carrier. The conjugate or compound in a pharmaceutically acceptable carrier is also referred to as pharmaceutical composition or composition.

A pharmaceutically acceptable carrier (excipient) is a pharmaceutically acceptable solvent, suspending emulsificant agent or any other pharmacologically inert vehicle for delivering the compound to an animal. The pharmaceutically acceptable carrier is for example liquid or solid and is selected with the planned manner of administration in mind so as to provide for the desired bulk, consistency, etc., when combined with a nucleic acid and the other components of a given pharmaceutical composition. Typical pharmaceutically acceptable carriers include, but are not limited to, binding agents (e.g. pregelatinized maize starch, polyvinylpyrrolidone or hydroxypropyl methylcellulose, etc.); fillers (e.g. lactose and other sugars, microcrystalline cellulose, pectin, gelatin, calcium sulfate, ethyl cellulose, polyacrylates or calcium hydrogen phosphate, etc.); lubricants (e.g., magnesium stearate, talcum, silica, colloidal silicon dioxide, stearic acid, metallic stearates, hydrogenated vegetable oils, corn starch, polyethyleneglycols, sodium benzoate, sodium acetate, etc.); disintegrates (e.g., starch, sodium starch glycolate, etc.); or wetting agents (e.g., sodium lauryl sulphate, etc.). Sustained release oral delivery systems and/or enteric coatings for orally administered dosage forms are described in U.S. Pat. Nos. 4,704,295; 4,556,552; 4,309,406; and 4,309,404. An adjuvant is included under these phrases.

Pharmaceutical compositions and formulations for topical administration include for example transdermal patches, pastes, ointments, lotions, creams, gels, drops, suppositories, globuli, sprays, liquids, and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions, emulsions, or solutions in water or non-aqueous media, capsules, sachets or tablets. In certain embodiments thickeners, flavoring agents, diluents, emulsifiers, dispersing aids or binders are used.

In other embodiments the pharmaceutical composition for parenteral, intrathecal, intracerebral (i.c.), or intraventricular administration include for example sterile aqueous solutions which in some embodiments contains buffers, diluents and other suitable additives such as penetration enhancers, carrier compounds and other pharmaceutically acceptable carriers or excipients.

In other embodiments the pharmaceutical composition also includes for example penetration enhancers in order to enhance the alimentary delivery of the oligonucleotide, protein and/or peptide. Penetration enhancers are for example classified as belonging to one of five broad categories, i.e., fatty acids, bile salts, chelating agents, surfactants and non-surfactants (Lee (1991), Muranishi (1990). One or more penetration enhancers from one or more of these broad categories are for example included in compositions according to the invention.

Various fatty acids and their derivatives which act as penetration enhancers include, for example, oleic acid, lauric acid, capric acid, myristic acid, palmitic acid, stearic acid, linoleic acid, linolenic acid, dicaprate, tricaprate, ricinoleate, monoolein (1-monooleoyl-rac-glycerol), dilaurin, caprylic acid, arachidonic acid, glyceryl 1monocaprate, 1-dodecylazacycloheptan-2-one, acylcarnitines, acylcholines, mono- and di-glycerides and physiologically acceptable salts thereof (i.e., oleate, laurate, caprate, myristate, palmitate, stearate, linoleate, etc.) (Lee (1991), Muranishi (1990), El-Hariri (1992). Examples of some presently preferred fatty acids are sodium caprate and sodium laurate, used singly or in combination at concentrations of 0.5 to 5%.

The physiological roles of bile include the facilitation of dispersion and absorption of lipids and fat-soluble vitamins (Brunton 1996). Various natural bile salts, and their synthetic derivatives, act as penetration enhancers. Thus, the term “bile salt” includes any of the naturally occurring components of bile as well as any of their synthetic derivatives. A presently preferred bile salt is chenodeoxycholic acid (CDCA) (Sigma Chemical Company, St. Louis, Mo.), generally used at concentrations of 0.5 to 2%.

In particular, complex formulations comprising one or more penetration enhancers are used. For example, bile salts are used in combination with fatty acids to make complex formulations. Preferred combinations include CDCA combined with sodium caprate or sodium laurate (generally 0.5 to 5%).

Chelating agents include, but are not limited to, disodium ethylenediaminetetraacetate (EDTA), citric acid, salicylates (e.g., sodium salicylate, 5-methoxysalicylate and homovanilate). N-acyl derivatives of collagen, laureth-9 and N-amino acyl derivatives of beta-diketones (enamines) (Lee (1991)), Muranishi (1990), Buur (1990). Chelating agents have for example the additional advantage of also serving as RNase inhibitors.

Surfactants include, for example, sodium lauryl sulfate, polyoxyethylene-9-lauryl ether and polyoxyethylene-20-cetyl ether (Lee (1991)), and perfluorochemical emulsions, such as FC-43 (Takahashi (1988)).

Non-surfactants include, for example, unsaturated cyclic ureas, 1-alkyl- and 1-alkenylazacyclo-alkanone derivatives (Lee (1991) and non-steroidal anti-inflammatory agents such as diclofenac sodium, indomethacin and phenylbutazone (Yamashita (1987)).

Regardless of the method by which the compounds of the invention are introduced into a patient, colloidal dispersion systems may be used as delivery vehicles to enhance the in vivo stability of the compounds and/or to target the compounds to a particular organ, tissue or cell type. Colloidal dispersion systems include, but are not limited to, macromolecule complexes, nanocapsules, nanospheres, microcapsules, microspheres, beads and lipid-based systems including oil-in-water emulsions, micelles, mixed micelles, liposomes and lipid-compound complexes of uncharacterized structure. A preferred colloidal dispersion system is a plurality of liposomes. Liposomes are microscopic spheres having an aqueous core surrounded by one or more outer layer(s) made up of lipids arranged in a bilayer configuration (Chonn (1995)).

In some embodiments of a pharmaceutical composition of this invention where an acid form exists, salt and ester forms are preferably formed from the acid, and all such forms are included within the meaning of the terms conjugate, compound or oligonucleotide, protein and/or peptide as used herein. Pharmaceutically acceptable salts shall mean non-toxic salts of the compounds employed in this invention which are generally prepared by reacting the free acid with a suitable organic or inorganic base, particularly those formed from cations such as sodium, potassium, aluminium, calcium, lithium, magnesium, zinc, and tetramethylammonium, as well as those salts formed from amines such as ammonia, ethylenediamine, N-methylglucamine, lysine, arginine, ornithine, choline. N,N′-dibenzylethylenediamine, chloroprocaine, diethanolamine, procaine. N-benzylphenethylamine, 1-p-chlorobenzyl-2-pyrrolidine-1′-yl-methylbenzimidazole, diethylamine, piperazine, and tris(hydroxymethyl)aminomethane.

### Therapy

The oligonucleotide, protein and/or peptide, e.g., an immunostimulator, linked with at least one polymer or a pharmaceutical composition comprising one or more conjugates or compounds of the present invention is suitable for controlling, preventing and/or treating diseases or disorders. In preferred embodiments the conjugate and compound, respectively, or the pharmaceutical composition is suitable for controlling, preventing and/or treating unwanted neoplasms such as cancer, carcinoma, fibrosis and/or eye diseases or disorders as well as for controlling, preventing and/or treating of a viral disease or disorder such as HIV. Further the invention is directed to the use of a conjugate or compound, or of a pharmaceutical composition according to the present invention for controlling, preventing and/or treating a disease or disorder, wherein the disease or disorder is selected from the group of cancer, fibrosis, and viral disease or disorder.

In a preferred example the pharmaceutical composition comprises at least one oligonucleotide, protein and/or peptide, e.g., an immunostimulator linked with at least one polymer. Preferably, the pharmaceutical composition is produced by a method, wherein the conjugate or compound is produced according to a method of the present invention and the addition of a pharmaceutically acceptable carrier. Optionally, the pharmaceutical composition further comprises an active substance, a chelating agent, a surfactant, a fatty acid, a penetration enhancer, an emulsificant agent, a lubricant, etc.

The neoplasms, cancers or carcinomas controllable, preventable and/or treatable with an oligonucleotide, a protein and/or a peptide for example an immunostimulator comprising at least one oligonucleotide, protein and/or peptide, e.g., an immunostimulator, linked with at least one polymer or a pharmaceutical composition comprising or consisting of such conjugate include, but are not limited to solid tumors, blood born tumors such as leukemias, acute or chronic myelotic or lymphoblastic leukemia, tumor metastasis, benign tumors, for example hemangiomas, acoustic neuromas, neurofibromas, trachomas, pyogenic granulomas, pre-malignant tumors, rheumatoid arthritis, psoriasis, astrocytoma, acoustic neuroma, blastoma, craniopharyngioma, ependymoma. Ewing's tumor, medulloblastoma, glioma, hemangioblastoma. Hodgkins-lymphoma, medulloblastoma, leukaemia, mesothelioma, neuroblastoma, neurofibroma, non-Hodgkins lymphoma, pinealoma, retinoblastoma, sarcoma (including angiosarcoma, chondrosarcoma, endothelialsarcoma, fibrosarcoma, leiomyosarcoma, liposarcoma, lymphangioendotheliosarcoma, lymphangiosarcoma, melanoma, meningioma, myosarcoma, oligodendroglioma, osteogenic sarcoma, osteosarcoma), seminoma, trachomas. Wilm's tumor, or is selected from the group of bile duct carcinoma, bladder carcinoma, brain tumor, breast cancer, bronchogenic carcinoma, carcinoma of the kidney, cervical cancer, choriocarcinoma, cystadenocarcinoma, embryonal carcinoma, epithelial carcinoma, esophageal cancer, cervical carcinoma, colon carcinoma, colorectal carcinoma, endometrial cancer, gallbladder cancer, gastric cancer, head cancer, liver carcinoma, lung carcinoma, medullary carcinoma, neck cancer, non-small-cell bronchogenic/lung carcinoma, ovarian cancer, pancreas carcinoma, papillary carcinoma, papillary adenocarcinoma, prostate cancer, small intestine carcinoma, prostate carcinoma, rectal cancer, renal cell carcinoma, skin cancer, small-cell bronchogenic/lung carcinoma, squamous cell carcinoma, sebaceous gland carcinoma, testicular carcinoma, uterine cancer.

The conjugates of the invention as well as the pharmaceutical compositions comprising one or more such conjugates are also suitable to control, prevent and/or treat a variety of immune disorders such as autoimmune diseases or disorders, e.g., diabetes mellitus, arthritis, including rheumatoid arthritis, juvenile rheumatoid arthritis, osteoarthritis, psoriatic arthritis; multiple sclerosis, encephalomyelitis, myasthenia gravis, systemic lupus erythematosis, autoimmune thyroiditis, dermatitis, including atopic dermatitis, eczematous dermatitis; psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis. Wegener's granulomatosis, chronic active hepatitis. Stevens-Johnson syndrome, idiopathic sprue, lichen planus. Graves' disease, sarcoidosis, primary biliary cirrhosis, uveitis posterior, and interstitial lung fibrosis, graft-versus-host disease, cases of transplantation, and allergy such as an atopic allergy. The conjugate or pharmaceutical compositions are also suitable to control, prevent and/or treat cardiovascular diseases or disorders such as hypertension, atherosclerosis, coronary artery spasm, congestive heart failure, coronary artery disease, valvular disease, arrhythmias, and cardiomyopathies; or viral diseases or disorders for example hepatitis A (HVA), hepatitis B (HVB), hepatitis C (HVC), or caused by herpes simplex virus (HSV), HIV, FIV, poliovirus, influenza virus, adenoviruses, papillomaviruses. Epstein-Barr-viruses and small pox virus, or virus-associated cancer such as hepatocellular cancer.

The conjugates of the invention as well as the pharmaceutical compositions comprising one or more such conjugates are further suitable to control, prevent and/or treat fibrotic diseases or disorders which are for example associated with undesired TGF-beta signaling, which include, without limitation, kidney disorders and (excessive) fibrosis and/or sclerosis, such as glomerulonephritis (GN) of all etiologies, e.g., mesangial proliferative GN, immune GN, crescentic GN; diabetic nephropathy, renal interstitial fibrosis and all causes of renal interstitial fibrosis including hypertension, renal fibrosis resulting from complications of drug exposure, including cyclosporin treatment of transplant recipients. HIV-associated nephropathy, or transplant nephropathy; hepatic diseases associated with (excessive) scarring and (progressive) sclerosis for example cirrhosis due to all etiologies, disorders of the biliary tree, and hepatic dysfunction; pulmonary fibrosis with consequential loss of gas exchange or ability to efficiently move air into and out of the lungs such as adult respiratory distress syndrome (ARDS), chronic obstructive pulmonary disease (COPD), idiopathic pulmonary fibrosis (IPF), acute lung injury (ALI) or pulmonary fibrosis due to infectious or toxic agents such as smoke, chemicals, allergens, or autoimmune diseases; eye diseases or disorders associated with fibroproliferative states such as fibroproliferative vitreoretinopathy of any etiology or fibrosis associated with ocular surgery, e.g., treatment of glaucoma, retinal reattachment, cataract extraction, or drainage procedures of any kind; excessive or hypertrophic scar formation in the dermis occurring for example during wound healing resulting from trauma or surgical wounds.

The following examples will serve to further illustrate the present invention without, at the same time, however, constituting any limitation thereof. To the contrary, it is to be clearly understood that resort may be had to various other embodiments, modifications, and equivalents thereof which, after reading the description herein, may suggest themselves to those skilled in the art without departing from the spirit of the invention. In particular, methods described for the modification of the 5′- or the 3′-terminus of the oligonucleotide, the N-terminus or the C-terminus of the protein and/or peptide are combinable for the production of conjugates modified on both ends. Additionally, methods disclosed for the production of 5′3′- or N-/C-terminal-modified conjugates are variable in a way that only one end is modified or one end and a sugar moiety, a base, a NH— or SH-group etc. is modified.

### EXAMPLES

### Example 1

### Synthesis of a 5′-PEGylated Oligonucleotide

### 1) Synthesis of DMT-PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000

### DMT=Dimethoxytrityl

PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750 or -5000, -10000, -20000, -50000 or -1000000 (20.0 mmol) was evaporated twice from 5 ml pyridine (dried with molecular sieve) and dried in vacuo 4 days over P₄O₁₀. The PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000 was dissolved in dichloromethane (5 ml, refluxed and distilled with CaH₂) and triethylamine (7.21 mmol, refluxed and distilled with CaH₂) and 4-dimethyl-aminopyridine (0.21 mmol) were added. Subsequently 4,4′-dimethoxytriphenylmethyl chloride (4.5 mmol) was added within 1 hour and the mixture was stirred for 24 hours in the dark. The mixture was diluted with 50 ml dichloromethane and extracted twice with 25 ml 5% sodium hydrogencarbonate and once with 25 ml water. The organic phase was dried (Na₂SO₄) and concentrated by rotary evaporation.

The mixture was purified by column chromatography silica gel 60 (diameter 0.063-0.100 mm) with the solvent:chloroform:methyl alcohol:triethylamine (96.5:3:0.5). The product was identified by NMR.

2) Synthesis of DMT-PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000-phosphoramidite:

DMT-PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000 (0.41 mmol) was evaporated twice from 30 ml pyridine (dried with molecular sieve)/dichloromethane (refluxed and distilled with CaH₂) at a ratio of 1:10 and dried in vacuo 6 days over P₄O₁₀. The DMT-PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000 was dissolved in dichloromethane (970 μl, refluxed and distilled with CaH₂) and diisopropylethylamine (1.65 mmol, refluxed and distilled with CaH₂) and 2-cyanoethyl-diisopropylchlorophosphoramidite (0.63 mmol) was added. The mixture was stirred for 30 minutes in the dark (reaction detection by thin layer chromatography (TLC): silica gel 60 F₂₅₄, dichloromethane/ethyl acetate/triethylamine, 45/45/10). Methyl alcohol (20 μl, distilled with magnesium) was added. The mixture was diluted with 6 ml dichloromethane and extracted twice with 10 ml 5% sodium hydrogencarbonate and twice with 10 ml distilled water. The organic phase was dried (Na₂SO₄) and concentrated by rotary evaporation.

The mixture was purified by chromatography (silica gel 60 (0.040-0.063 mm); dichloromethane/ethyl acetate/triethylamine, 45/45/10). The product was identified by NMR.

3) Synthesis of 5′-PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000-oligonucleotide conjugates:

The oligonucleotide was synthesized by standard phosphoramidite chemistry on 1000 Å adenosine-CPG (10 micromol, loading 38 micromol/g support using a Expedite Synthesizer (PerSeptive Biosystems). The 5′-OH of the oligonucleotide was PEGylated by using the DMT-PEG 400-phosphoramidite (50 mg/ml acetonitrile) on the Synthesizer. In order to calculate the loading of DMT-groups, an aliquote was taken, 0.1M toluene-4-sulfonic acid was added and the absorption of the cleavaged DMT-cations were determinated at 260 nm (loading: 11.2 μmol). The product was cleaved from the support and deprotected by using 32% NH₃ at 55° C. for 16 h. The crude product was dried and purified by reversed-phase HPLC (TOSOHAAS, Amberchrom CG-300S (reversed phase), 35 μm; 125×4.7 mm; acetonitrile /0.1 M triethylammonium acetate pH 7.5). The oligonucleotide was detritylated by using 80% acetic acid at room temperature for 15 min, desalted by using a NAP-10-Column (Amersham Biosciences) and lyophilized.

The conjugates resulting from different experiments were: PEG 400-oligonucleotide, PEG 600-oligonucleotide, PEG 800-oligonucleotide, PEG 1000-oligonucleotide, PEG 1500-oligonucleotide, PEG 2000-oligonucleotide, PEG 5000-oligonucleotide, PEG 20000-oligonucleotide, and PEG 50000-oligonucleotide.

### Analysis:

a) MALDI-TOF: Matrix: 3-hydroxypicolinic acid, reflector-mode, molecular weight was determined
b) Capillary gel electrophoresis: capillary: fused silica capillary, 40 cm, inner diameter: 100 μm, external diameter: 360 μm, buffer: 10% buffer solution pH 2.5 for HPCE (0.1 M sodium phosphate-buffer) and 90% 0.1% (hydroxypropyl)methyl cellulose in water, sample: 10 pmol oligonucleotide/μl (in water), voltage: 8 kV, polarity: from − to +, run time: 30 min, run: capillary was rinsed under pressure, 60 sec with 0.1M NaOH (filtered 0.2 μm), 120 sec with buffer, sample injection (5 sec. with pressure), results: broad peak at 20.5 min

### Example 2

### Synthesis of a 5′-PEGylated Oligonucleotide (FIG. 8)

### 1) Oligonucleotide Synthesis:

Oligonucleotides were synthesized by phosphoramidite chemistry, wherein the 5′-terminus of the oligonucleotide is aminofunctionalized, i.e., the 5′-terminus comprises a functional group which is an amino group.

### 2) Synthesis of NHS-Ester PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000:

The NHS-ester PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750-5000, -10000, -20000, -50000 or -1000000 is producable according to any method known in the art.

### 3) Synthesis of 5′-PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000-Oligonucleotide Conjugates:

The 5′-terminus aminofunctionalized oligonucleotide is dissolved in reaction buffer (60% 0.3 M NaHCO₃, pH 8.5/40% DMF), for example 1 μmol oligonucleotide in 1.6 ml reaction buffer (0.625 μmol/ml), and warmed up to 37° C. In the following 28 μmol NHS-ester PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000 is added step by step within 2 h to the 37° C. oligonucleotide. After all the NHS-ester is added to the oligonucleotide, the sample is incubated for 16 h at 37° C. on a shaker (100 rpm). In the following the sample is dried in a SpeedVac.

a) Alternative I: The dried sample is dissolved in 1 ml 0.01 M TEAA pH 7.5 and purified on an ion exchange FPLC (IE-FPLC) to remove the hydrolyzed NHS and PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000. A suitable ion exchange column is for example Toyopearl SuperQ-650M 150×16 mm, the elution buffer is 0.01 M TEAA+2 M NaCl pH 7.5, and the flow rate is 4 ml/min. Afterwards the sample is desalted via ultrafiltration using an ultrafiltration membrane of MWCO 1000 Da and a pressure of 4 bar N₂ to reduce or remove the NaCl concentration. The desalted sample is dried in a SpeedVac at room temperature.
b) Alternative II: The dried sample is dissolved in 1 ml 0.01 M TEAA pH 7.5 and purified via reversed-phase HPLC to remove the hydrolyzed NHS and PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000. The column used is a Phenomenex Polymerx RP1 100 Å 10×250 mm, which is equilibrated with 0.1 M TEAA pH 7.5, and the sample is eluted with acetonitril. The sample comprising the PEG conjugated oligonucleotide is dried in a SpeedVac and afterwards the dried sample is dissolved in 100 μl 1 M sodium acetate solution and 1.9 ml water. The sample is finally dried again in a SpeedVac.
c) Alternative III: After all the NHS-ester is added to the oligonucleotide, the sample is incubated for 16 h at 37° C. on a shaker (100 rpm); in the following the volume of the sample is reduced to 0.5 ml in a SpeedVac to reduce the DMF content. 2.5 ml water is added to the 0.5 ml sample, which is then purified via IE-FPLC (see alternative I). The buffer system used for alternative III is buffer A: 0.01 M NaHCO₃ pH 8.5 and buffer B: 0.001 M NaHCO₃+2 M NaCl pH 8.5 using a flow rate of 4 ml/min Afterwards the sample comprising the PEG conjugated oligonucleotide is desalted via ultrafiltration using an ultrafiltration membrane of MWCO 1000 Da and a pressure of 3.2 bar Ar to reduce or remove the NaCl and NaHCO₃ concentration. Finally, the sample is dried in a SpeedVac.

### Analysis:

MALDI-TOF: Matrix: 3-hydroxypicolinic acid, reflector-mode, molecular weight was determined.

### Example 3

### Synthesis of a 5′-PEGylated Oligonucleotide via PEG-Amidite

### 1) Oligonucleotide Synthesis:

The oligonucleotide is synthesized by phosphoramidite chemistry, wherein the 5′-terminus of the oligonucleotide is DMT protected.

2) Synthesis of PEG-200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000-Amidite: PEG-Amidite is Commercially Available.

### 3) Synthesis of 5′-PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000-Oligonucleotide Conjugates:

Once the oligonucleotide is synthesized on the CPG support, the PEG-amidite is added and a further coupling cycle is performed on the oligonucleotide synthesizer. Afterwards the PEGylated oligonucleotide is cleaved from the support by incubation of the conjugate with 700 μl ammoniac for 16 h at 40° C. In the following the conjugate is dried in a SpeedVac and the pellet is dissolved in 200 μl 0.1 M TEAA pH 7.5 and is finally purified via reverse phase HPLC (Phenomenex Jupiter 5μ C18 column 300 Å 4.6×250 mm, buffer A: 0.1 M TEAA pH 7.5, buffer B: acetonitrile). The conjugate is dried in a SpeedVac, desalted and dried again.

### Example 4

### Synthesis of a 3′-PEGylated Oligonucleotide

### 1) Succinylation of Controlled Pore Glass (CPG) Support:

Succinic anhydride (44 μmol) and 4-dimethylaminopyridine (22 μmol) were dissolved in pyridine (440 μl). The mixture was added to aminopropylated CPG support (4.4 μmol) and mixed at room temperature. After 20 h the solvent was removed and the CPG-support was washed five times with 1 ml dichloromethane and three times with 1 ml acetonitrile. For testing of unreacted amino group, an aliquot (approx. 1 mg) of CPG-support was taken and 50 μl of a ninhydrin-solution (50 mg ninhydrin/ml ethanol) was added. After 10 minutes at 55° C. no colour was detected.

### 2) PEGylation of the Succinylated CPG Support:

2-(1H-benzotriazole-1-yl)-1,1,3,3-tetramethyluronium hexafluorophosphate (HBTU, 1.5 mmol), 4-dimethylaminopyridine (1.5 mmol) and diisopropylethylamine (1.5 mmol) were dissolved in acetonitrile (11.25 ml). The mixture was added to the succinylated CPG support (combined, see 1); approx. 13.2 μmol). Subsequently PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000 (6.36 mmol) was added and the mixture was mixed at room temperature. After 20 h the solution was removed and the CPG-support was washed three times with 15 ml dichloromethane and three times with 15 ml acetonitrile.

### 3) Oligonucleotide Synthesis:

Oligonucleotides were synthesized by phosphoramidite chemistry on the PEGylated-CPG support using a Expedite Synthesizer. The product was cleaved from the support and deprotected by using 32% NH₃ at 55° C. for 16 h. The crude product was dried and purified by reversed-phase HPLC (TOSOHAAS, Amberchrom CG-300S (reversed phase), 35 μm; 125×4.7 mm; acetonitrile/0.1M triethylammonium acetate pH 7.5). The oligonucleotide was detritylated by using 80% acetic acid at room temperature for 15 min, desalted by using a NAP-10-Column (Amersham Biosciences) and lyophilized.

The conjugates resulting from different experiments were: 3′-PEG 400-oligonucleotide, 3′-PEG 600-oligonucleotide, 3′-PEG 800-oligonucleotide, K-PEG 1000-oligonucleotide, 3′-PEG 1500-oligonucleotide, 3′-PEG 2000-oligonucleotide, 3′-PEG 5000-oligonucleotide, 3′-PEG 20000-oligonucleotide, and 3′-PEG 50000-oligonucleotide.

### Analysis:

a) MALDI-TOF: Matrix: 3-hydroxypicolinic acid, reflector-mode, molecular weight was determined
b) capillary gel electrophoresis: capillary: fused silica capillary, 40 cm, inner diameter: 100 μm, external diameter: 360 μm, buffer: 10% buffer solution pH 2.5 for HPCE (0.1 M sodium phosphate buffer), 90%, 0.1% (hydroxypropyl)methyl cellulose in water voltage: 8 kV; polarity: from − to +; run time: 30 min; concentration of the sample: about 10 pmol oligonucleotide/μl (in water), Method: I) capillary preparation: capillary was rinsed under pressure: 360 sec with water (distilled, filtered pore diameter 0.2 μm), 120 sec. with 0.1 M NaOH (filtrated pore diameter 0.2 μm), 360 sec with water (distilled, filtrated pore diameter 0.2 μm)
II) run: capillary was rinsed under pressure: 60 sec. with 0.1M NaOH (filtrated 0.2 μm) 120 sec. with buffer; sample injection (5 sec. with pressure); result: broad peak at 20 min.

### Example 5

### Synthesis of a 3′-PEGylated Oligonucleotide

### 1) Oligonucleotide Synthesis:

The oligonucleotide is synthesized on a PT 3′-aminomodifier C6 CPG or 3′-aminomodifier C7 CPG, for example of Glen, or Amino On CPG, for example of Proligo (which all comprise an amino linker). The synthesis protocol is the standard base coupling protocol for thiolates. Once the oligonucleotide is synthesized, the conjugate is cleaved from the support by incubation with 700 μl ammoniac for 12 h at 40° C. Afterwards the sample is dried in a SpeedVac and the pellet is resolved in 100 μl water. The sample is then purified via a reverse phase HPLC (Phenomenex Jupiter 5μ C18 column 300 Å 4.6×250 mm, buffer A: 0.1 M TEAA pH 7.5, buffer B: acetonitrile) and dried again. In the following the sample is resolved again in 200 μl 80% acetic acid, desalted via a NAP column and dried in a SpeedVac.

### 2) Synthesis of 3′-PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000-Oligonucleotide Conjugates:

The purified 3′-aminomodified oligonucleotide is incubated with 12.5× molar excess of PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750 or -5000, -10000, -20000, -50000 or -1000000-NHS in reaction buffer (DMF/NaHCO₃ pH 8.5, 40:60) or 20 μl DMF/1 μl DIEA). 180 μl water are added to stop the reaction and the sample is purified via reverse phase HPLC (Phenomenex Jupiter 5μ C18 column 300 Å 4.6×250 mm, buffer A: 0.1 M TEAA pH 7.5, buffer B: acetonitrile). Finally the sample is lyophilized.

### Example 6

### Synthesis of a 3′5′-PEGylated Oligonucleotide

### 1) Succinylation of Controlled Pore Glass (CPG) Support:

Succinic anhydride (44 μmol) and 4-di(methyl)aminopyridine (22 μmol) were dissolved in pyridine (440 μl). The mixture was added to aminopropylated CPG-support (4.4 μmol, loading: 44 μmol) and mixed at room temperature. After 22 h the solution was removed and the CPG-support was washed five times with 1 ml dichlormethane and three times with 1 ml acetonitrile. For testing for unreacted amino groups, an aliquot (approx. 1 mg) of CPG-support was taken and 50 μl of a ninhydrin-solution (50 mg ninhydrin/ml ethanol) was added. After 10 minutes at 55° C. no colour was detected indicating no free amino groups.

### 2) PEGylation of the Succinylated CPG Support:

2-(1H-benzotriazole-1-yl)-1,1,3,3-tetramethyluronium hexafluorophosphate (HBTU, 3.0 mmol), 4-di(methyl)aminopyridine (3.0 mmol) and diisopropylethylamine (3.0 mmol) were dissolved in acetonitrile (22.5 ml). The mixture was added to the succinylated CPG-support (approx. 26.4 μmol), Subsequently, PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000 (12.7 mmol) was added and the mixture was mixed at room temperature. After 24 h the solvent was removed and the CPG support was washed three times with 10 ml dichloromethane and three times with 10 ml acetonitrile.

### 3) 3′-PEGylated Oligonucleotide Synthesis:

Oligonucleotides were synthesized according to phosphoramidite chemistry on the PEGylated-CPG support using a Expedite Synthesizer. For more details see also R. Schlingensiepen (1997). In order to calculate the loading of DMT-groups, an aliquote was taken, 0.1M toluene-4-sulfonic acid was added and the absorption of the cleavaged DMT-cations was determinated at 260 nm (loading: 16.8 μmol).

### 4) 5′-PEGylation of the Oligonucleotides:

The oligonucleotides were PEGylated by using DMT-PEG 200, -300, -400, -500, -600, -700, -800, -900, -1000, -1250, -1500, -1750, -2000, -2250, -2500, -2750, -3000, -3250, -3500, -3750, -4000, -4250, -4500, -4750, -5000, -10000, -20000, -50000 or -1000000-phosphoramidite in acetonitrile (dry) on the Expedite Synthesizer. For calculating the loading of DMT-groups, an aliquote was taken, 0.1M toluene-4-sulfonic acid was added and the absorption of the cleavaged DMT-cations was determinated at 260 nm (loading: 6.3 μmol).

The product was cleaved from the support and deprotected by using 32% NH₃ at 55° C. for 16 h. The crude product was dried and purified by reversed-phase HPLC (TOSOHAAS, Amberchrom CG-300S (reversed phase), 35 μm; 125×4.7 mm; acetonitrile/0.1 M triethylammonium acetate pH 7.5). The oligonucleotide was detritylated by using 80% acetic acid at room temperature for 15 min, desalted by using a NAP-10-Column (Amersham Biosciences) and lyophilized.

Preferred final products in different experiments were 3′5′- or 5′3′-PEGylated oligonucleotides having the following structure: PEG 200-oligonucleotide-PEG 200, PEG 300-oligonucleotide-PEG 300, PEG 400-oligonucleotide-PEG 400, PEG 500-oligonucleotide-PEG 500, PEG 600-oligonucleotide-PEG 600, PEG 700-oligonucleotide-PEG 700, PEG 800-oligonucleotide-PEG 800. PEG 900-oligonucleotide-PEG 900, PEG 1000-oligonucleotide-PEG 1000, PEG 1250-oligonucleotide-PEG 1250, PEG 1500-oligonucleotide-PEG 1500, PEG 1750-oligonucleotide-PEG 1750, PEG 2000-oligonucleotide-PEG 2000, PEG 2250-oligonucleotide-PEG 2250. PEG 2500-oligonucleotide-PEG 2500, PEG 2750-oligonucleotide-PEG 2750. PEG 3000-oligonucleotide-PEG 3000, PEG 3250-oligonucleotide-PEG 3250. PEG 3500-oligonucleotide-PEG 3500, PEG 3750-oligonucleotide-PEG 3750. PEG 4000-oligonucleotide-PEG 4000, PEG 4250-oligonucleotide-PEG 4250. PEG 4500-oligonucleotide-PEG 4500, PEG 4750-oligonucleotide-PEG 4750, PEG 5000-oligonucleotide-PEG 5000, PEG 200-oligonucleotide-PEG 500, PEG 200-oligonucleotide-PEG 600, PEG 200-oligonucleotide-PEG 700, PEG 200-oligonucleotide-PEG 800, PEG 200-oligonucleotide-PEG 900, PEG 200-oligonucleotide-PEG 1000, PEG 300-oligonucleotide-PEG 500, PEG 300-oligonucleotide-PEG 600, PEG 300-oligonucleotide-PEG 800. PEG 300-oligonucleotide-PEG 1000, PEG 300-oligonucleotide-PEG 1500, PEG 300-oligonucleotide-PEG 2000, PEG 400-oligonucleotide-PEG 600, PEG 400-oligonucleotide-PEG 800, PEG 400-oligonucleotide-PEG 1000, PEG 400-oligonucleotide-PEG 1500. PEG 400-oligonucleotide-PEG 2000, PEG 400-oligonucleotide-PEG 5000, PEG 400-oligonucleotide-PEG 20000, PEG 400-oligonucleotide-PEG 50000, PEG 500-oligonucleotide-PEG 800, PEG 500-oligonucleotide-PEG 1000, PEG 500-oligonucleotide-PEG 1250. PEG 500-oligonucleotide-PEG 1500, PEG 500-oligonucleotide-PEG 1750, PEG 500-oligonucleotide-PEG 2000, PEG 500-oligonucleotide-PEG 2250, PEG 500-oligonucleotide-PEG 2500, PEG 500-oligonucleotide-PEG 5000, PEG 600-oligonucleotide-PEG 800. PEG 600-oligonucleotide-PEG 1500, PEG 600-oligonucleotide-PEG 2000, PEG 600-oligonucleotide-PEG 5000, PEG 600-oligonucleotide-PEG 20000, PEG 600-oligonucleotide-PEG 50000, PEG 800-oligonucleotide-PEG 1500, PEG 800-oligonucleotide-PEG 2000. PEG 800-oligonucleotide-PEG 5000, PEG 800-oligonucleotide-PEG 20000, PEG 800-oligonucleotide-PEG 50000, PEG 1000-oligonucleotide-PEG 2000, PEG 1000-oligonucleotide-PEG 5000, PEG 1000-oligonucleotide-PEG 20000, PEG 1000-oligonucleotide-PEG 50000. PEG 1000-oligonucleotide-PEG 5000, PEG 1500-oligonucleotide-PEG 20000. PEG 1500-oligonucleotide-PEG 50000, PEG 2000-oligonucleotide-PEG 5000. PEG 2000-oligonucleotide-PEG 20000, PEG 2000-oligonucleotide-PEG 50000. PEG 2000-oligonucleotide-PEG 75000, PEG 2000-oligonucleotide-PEG 100000. PEG 5000-oligonucleotide-PEG 20000, PEG 5000-oligonucleotide-PEG 50000. PEG 5000-oligonucleotide-PEG 75000 or PEG 5000-oligonucleotide-PEG 1000000.

### Analysis:

a) MALDI-TOF: Matrix: 3-hydroxypicolinic acid, reflector-mode, molecular weight was determined
b) Capillary gel electrophoresis: capillary: fused silica capillary, 40 cm, inner diameter: 100 μm, external diameter: 360 μm, buffer: 10% buffer solution pH 2.5 for HPCE (0.1 M sodium phosphate buffer), and 90% 0.1% (hydroxypropyl)methyl cellulose in water; sample: 10 pmol oligonucleotide/μl (in water); voltage: 8 kV; polarity: from − to +; run time: 30 min; run: capillary was rinsed under pressure, 60 sec with (0.1M NaOH (filtered 0.2 μm), 120 sec with buffer, sample injection (5 sec with pressure), results: broad peak at 21 min

### Example 7

### Synthesis of a 3′5′-PEGylated Oligonucleotide via Conjugation in Solution

### 1) Oligonucleotide Synthesis:

Oligonucleotides were synthesized by phosphoramidite chemistry, the standard CPG method, wherein the 3′- and 5′-termini of the oligonucleotide is aminofunctionalized, i.e., the 5′-terminus comprises a functional group, which is an amino group. The aminofunctionalized 5′-end of the oligonucleotide comprising an amino linker at the 5′-terminus is temporarily protected by a trityl group for example monomethoxytrityl (MMT).

### 2) Synthesis of NHS-Ester PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000:

The NHS-ester PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000 is producable according to any method known in the art.

### 3) Synthesis of 3′-PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000-Oligonucleotide Conjugates:

The 5′-terminus protected oligonucleotide (21 nmol) is incubated with a 24 times excess of NHS-ester PEG 200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000 in 100 μl reaction buffer (60% 0.3 M NaHCO₃ pH 8.5/40% DMF). The sample is incubated for 17 h at 37° C. on a shaker (200 rpm). In the following the sample is optionally deprotected and dried in a SpeedVac and dissolved in 60 μl water. Afterwards the sample is purified by HPLC. Finally, the conjugate is desalted via ultrafiltration and dried in a SpeedVac.

### 4) 5′-PEGylation of the 3′-PEGylated Oligonucleotide:

In case the protecting group was not removed under step 3, the protecting group of the 5′-end is removed and purified. The 3′-PEGylated oligonucleotide is dissolved in water and is incubated with an excess of PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000-NHS-ester for 17 h at 40° C. Afterwards, the sample is purified via a HPLC and FPLC. In the following, the sample is desalted via ultrafiltration and dried in a SpeedVac.

### Example 8

### Synthesis of a 3′5′-PEGylated Oligonucleotide

### 3′-PEGylation on Support (FIG. 9)

### 1) Synthesis of NHS-Ester PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000:

The NHS-ester PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000 is producable according to any method known in the art.

### 2) Synthesis of 3′-PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000-Oligonucleotide Conjugates:

A further method to modify the oligonucleotide on the 3′- and the 5′-terminus is the use of 3′-amino-modifier C7 CPG of Glen Research. This modifier is a bifunctional support for oligonucleotide synthesis comprising a trityl group, for example DMT, in 5′-direction and a Fmoc protected amino group in 3′-direction. Both functional groups are connected via a C6-linker, which is finally connected to the CPG support via a succinate bridge. The Fmoc protected amino group is modified, i.e., connected to NHS-ester PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000 before the synthesis of the oligonucleotide. The Fmoc protection group is removed from the 3′-terminus of the C7 CPG support of Glen by incubating C7 CPG for 2×10 min and 2×15 min with 3 ml 20% piperidin in DMF. Afterwards the sample is washed 6× with 3 ml DMF and 3× with 3 ml ACN. Once Fmoc is removed, the support is dried under Ar, and a 15× to 30× excess of NHS-ester PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000 in dry DMF is added to the support. Diisopropylethylamine (10 μl; DIEA) is added to the sample. The sample is incubated for 2 h at 37° C. on a shaker (200 rpm). After the incubation the sample is washed 3× with 1 ml DMF, 3× with 1 ml ACN, and 3× with 1 ml DCM; finally the sample is dried at room temperature. To avoid negative effects on the oligonucleotide synthesis, potentially remaining free 3′-amino groups are capped by incubation of the support for 5 min at room temperature with “capping reagents” (for example acetic acid anhydride) of the oligonucleotide synthesizer. In the following the support is washed 3× with 1 ml ACN and finally dried at room temperature. Afterwards the oligonucleotide is synthesized including a 5′-aminolinker in a final cycle according to standard protocols for base coupling of thioates, wherein the trityl group (DMT) of the support is kept. The 3′-PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000 conjugated oligonucleotide is cleaved from the support by incubation in 200 μl conc. ammoniac for 20 h at 55° C. Finally, the sample is dried in a SpeedVac to remove the ammoniac. The pellet is resolved in 50 μl 0.1 M TEAA pH 7.5 and purified via reverse-phase HPLC to remove incomplete oligonucleotide fragments (column: Amberchrom CG-300S, buffer A: 0.1 M TEAA pH 7.5, buffer B: acetonitrile). Finally, the DMT protection group is removed from the 5′-terminus of the oligonucleotide by incubating the sample with 200 μl 80% acetic acid for 2 h at room temperature. The acetic acid and the removed DMT group are separated from the sample via a NAP column. The sample is dried in the SpeedVac and dissolved again in 200 μl water. The sample is purified via a Phenomenex PolymerX RP1 column 100 Å 10×250 mm (buffer A: 0.1 M TEAA pH 7.5, buffer B: acetonitrile). Finally the sample is concentrated by ultrafiltration (MWCO=1000 Da; pressure of 4 bar Ar), and is dried in a SpeedVac.

### 3) 5′-PEGylation of the 3′-PEGylated Oligonucleotide:

The 3′-PEGylated oligonucleotide is in a next step connected to NHS-ester PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000.

The product of step 2) is resolved in 150 μl reaction buffer (60% 0.3 M NaHCO₃ pH 8.5/40% DMF), 30× excess of NHS-ester PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000 is added, and the sample is incubated for 2 h at 37° C. on a shaker (200 rpm). Afterwards the sample is dried in a SpeedVac and resolved in 200 μl water. The sample is then purified via a Phenomenex Jupiter 5μ C18 column 300 Å 4.6×250 mm (buffer A: 0.1 M NaHCO₃ pH 8.5, buffer B: acetonitrile). The sample is desalted and concentrated by ultrafiltration (MWCO 1000 Da; pressure of 4 bar Ar) and dried again in a SpeedVac.

### Example 9

### Synthesis of a 3′5′-PEGylated Oligonucleotide on a Modified Support with PEG-Amidite

### 1) Synthesis of 3′-PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000-Oligonucleotide Conjugates:

The 3′-terminus of the oligonucleotide is modified according to the method described in Example 5 and 8, respectively.

### 2) 5′-PEGylation of the 3′-PEGylated Oligonucleotide:

Once the 3′-PEGylated oligonucleotide is synthesized. PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000-amidite is used in the final coupling cycle according to method of Example 3.

### Example 10

### Synthesis of a 3′5′-PEGylated Oligonucleotide

### 5′-PEGylation on Support (FIG. 10)

### 1) Oligonucleotide Synthesis:

The oligonucleotide is synthesized on a PT 3′-aminomodifier C6 CPG or 3′-aminomodifier C7 CPG, for example of Glen, or Amino On CPG, for example of Proligo (which all comprise an amino linker; cf. Example 5). The synthesis protocol is the standard base coupling protocol for thioates. In the last cycle of the synthesis the coupling protocol is extended for coupling a 5′-aminomodifier to the 5′-terminus of the oligonucleotide; such 5′-aminomodifier (linker) is for example DMS (O)MT-aminomodifier C6 or 5′-MMT-aminomodifier C5. After the oligonucleotide synthesis including the coupling of the 5′-aminomodifier, the trityl groups are removed by 30 cycles 3% TFA in DMF in the synthesizer.

### 2) Synthesis of 5′-PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000-Oligonucleotide Conjugates:

The detritylated sample is dried and 110 nmol of PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000-NHS in 30 μl DMF or ACN+1.5 μl DIEA is added. The sample is incubated for 4 h at 37° C. Afterwards the sample is washed 2× with 100 μl DMF and 2× with 100 μl ACN and dried. Then the 5′-PEGylated oligonucleotide is cleaved from the support by incubation of the sample with 200 μl conc. ammoniac for 17 h at 40° C. In the following the ammoniac is removed in the SpeedVac and the PEGylated oligonucleotide is dissolved in 200 μl water. Finally, the oligonucleotide is purified on a reverse phase HPLC (Amberchrom CG-300S, buffer A: 0.1 M TEAA pH 7.5, buffer B: acetonitrile).

### 3) 3′-PEGylation of the 5′-PEGylated Oligonucleotide:

150 to 350 nmol of PEG 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000-NHS, 30 to 50 μl DMF or ACN, and 1.5 to 2.5 μl DIEA are added to 5-35 nmol 5′-PEGylated oligonucleotide having a 3′-terminal amino linker (PT 3′-aminomodifier C6,3′-aminomodifier C7, or Amino On CPG), and the mixture is incubated for 1 h at 37° C. Finally, the conjugate is purified via reverse phase HPLC (Phenomenex Jupiter 5μ C18 column 300 Å 4.6×250 mm, buffer A: 0.1 M TEAA pH 7.5, buffer B: acetonitrile). Optionally, the conjugate is further purified via IE-FPLC in NaHCO₃ buffer and NaHCO₃+NaCl buffer. Finally, the conjugate is desalted via ultrafiltration (MWCO=1000 Da; pressure of 4 bar Ar), and dried in a SpeedVac.

### Analysis:

MALDI-TOF: Matrix: 3-hydroxypicolinic acid, reflector-Mode, molecular weight was determined.

### Example 11

### Synthesis of a 5′3″-PEGylated Oligonucleotide via 5′-Phosphoramidites (FIG. 11)

### 1) Oligonucleotide Synthesis:

The oligonucleotide synthesized according to this Example comprises PEG 1500, -2000, -5000, -20000 or -50000 on the 5′-terminal end and PEG 200, -400, -600, or -800 on the 3′-terminal end. The oligonucleotide is synthesized using specific 5′-phosphoric acid ester amides, where the position of the DMT protection group and the position of the phosphoric acid ester amide is opposite to the phosphoric acid ester amide used in general oligonucleotide synthesis methods. The general direction of oligonucleotide synthesis in a synthesizer is 3′ to 5′; 5′-phosphoramidites result in a direction of synthesis from 5′ to 3′, so that the 5′-terminal end is connected to the support. The oligonucleotide is synthesized in form of phosphorothioate. In a final step an aminomodifier (aminolinker) is linked to the 3′-terminal end of the oligonucleotide (cf. reaction conditions of Examples 5 and 10). Once the oligonucleotide is synthesized and the aminomodifier is linked (inverse linker), the sample is detritylated with 3% trichloro acetic acid in dichlor methan to remove the MMT- or DMS (O)MT-group from the 3′-terminal end. Finally, the oligonucleotide connected to the support is dried.

### 2) Synthesis of 3′-PEG 200, -400, -600, or -800-Oligonucleotide Conjugates:

70 μmol PEG 200, -400, -600, or -800-NHS are added to 14 μmol of the dried sample in 560 μl dry DMF. DIEA is used as support base, wherein 0.1 μl DIEA are added per μl oligonucleotide-PEG-mixture. The sample is incubated for 90 min at 37° C. on a shaker (100 rpm). Afterwards the sample is washed 5× with 1 ml acetonitrile to remove free PEG-NHS. The 3′-PEGylated oligonucleotide is cleaved from the support by incubation with 1 ml conc. Ammoniac for about 10 h at 55° C. In the following the sample is dried in a SpeedVac to remove the ammoniac and resolved in 50 μl water. The sample is purified via a reverse phase HPLC (Phenomenex Jupiter 5μ C18 column 300 A4.6×250 mm, buffer A: 0.1 M TEAA pH 7.5, buffer B: acetonitrile).

### 3) 5′-PEGylation of the 3′-PEGylated Oligonucleotide:

The purified 3′-PEGylated oligonucleotide is incubated with a 10× molar excess of PEG 1500, -2000, -5000, -20000 or -50000 in a reaction buffer comprising or consisting of DMF/0.3 M NaHCO₃, pH 8.5 (40:60). This mixture is incubated for 1 h at 37° C. on a shaker (200 rpm). Afterwards the sample is purified via a a reverse phase HPLC (Phenomenex Jupiter 5μ C18 column 300 Å 4.6×250 mm, buffer A: 0.1 M TEAA pH 7.5, buffer B: acetonitrile). In the following the volume of the sample is reduced in a SpeedVac, and the sample is washed with 200 μl water. Finally the sample is further purified via IE-FPLC (Toyopearl SuperQ-650M 150×16 mm, buffer A: 0.01 M NaHCO₃, pH 8.5, buffer B: 0.01 M NaHCO₃, pH 8.5+2 M NaCl). The sample is desalted via ultrafiltration (MWCO=1000 Da; pressure of 4 bar Ar), and dried in a SpeedVac.

### Analysis:

MALDI-TOF: Matrix: 3-hydroxypicolinic acid, reflector-Mode, molecular weight was determined.

### Example 12

### Synthesis of Oligonucleotide-PEG Conjugate in Single Reactor Unit

The oligonucleotide was synthesized by standard phosphoramidite chemistry on 1000 Å-CPG support using a Expedite Synthesizer (PerSeptive Biosystems). The 5′-OH of the oligonucleotide was aminomodified by a 2-[2-(4-monomethoxytrityl)aminoethoxy]ethyl-(2-cyanoethyl)-N,N-diisopropyl)-phosphoramidite (5′-amino-modifier 5, Glen Research, 0.067M in acetonitrile) on the Synthesizer. The product was cleaved from the support and deprotected by using 32% NH₃ at 40° C. for 17 h. The crude product was dried and purified by reversed-phase HPLC (Phenomenex, Polymerx, 10 μm, RP-1, 100 Å, 250×10 mm; 0.1M triethylammonium acetate pH 7.5/acetonitrile). The oligonucleotide was detritylated by using 80% acetic acid at room temperature for 1 h and eluted over a NAP-10-column (Amersham Biosciences).

The 5′-aminomodified oligonucleotide was dissolved in 0.3M NaHCO₃, pH 8.5, 40% DMF and heated to 37° C. The MPEG-SPA-5K (NEKTAR, 24 equivalents) was added stepwise. After 18 h the mixture was dried and the crude product was separated from the excess of PEG by ion-exchange chromatography (TOSOHAAS, Toyopearl, Super Q-650S, 250×4 mm; 0.01M triethylammonium acetate pH 7.5/0.01M triethylammonium acetate pH 7.5, 2 M NaCl). Subsequently, sodium chloride was removed by using Amicon Ultra Centrifugal Filter Devices (Millipore). The unreacted oligonucleotide was removed by reversed-phase HPLC (Phenomenex, Polymerx, 10 μm, RP-1, 100 Å, 250×10 mm; 0.1M triethylammonium acetate pH 7.5/acetonitrile). The purified product was desalted by using a NAP 10 column (Amersham Biosciences) and lyophilized.

### Analysis:

MALDI-TOF: Matrix: 3-hydroxypicolinic acid, reflector mode. Peak at approximately 11.5 kg/mol.

### Example 13

### Cell Culture Experiments

### Suppression of TGF-Beta2 Secretion

### Cell Culture:

The human glioblastoma cell line A-172 (Accession no. CRL-1620) was established from a 53-year old man with a glioblastoma. These cells were grown in monolayers. The A-172 tumor cells were cultivated in DMEM medium with 4.5 g/l glucose and 10% (v/v) FCS at 37° C., 5% CO₂, and 95% relative humidity. To investigate TGF-beta2 suppression as well as tumor cell proliferation, 10⁴ A-172 cells/well were seeded into 12-well tissue culture plates. 24 hours after seeding the supernatants were removed and replaced by a treatment solution consisting of cell culture medium containing the respective amount of oligonucleotide with linkers with at least one polymer. The exchange of the medium was repeated after 3 days (schedule signed as 2*3 days). Medium of the negative control (=untreated cells) and the medium control (=medium without cells) were changed without addition of oligonucleotide. After 6 days of treatment the supernatants were collected for quantification of secreted TGF-beta2 by using an Enzyme-Linked Immunosorbent Assay (ELISA) from R&D Systems. In parallel, the number of tumor cells was investigated by cell counting using an electronic cell counter.

### Methods:

TGF-beta2 in cell culture supernatants was quantified by using the Quantikine® Human TGF-beta2 Immunoassay according to the manufacturer's instructions (R&D Systems). The supernatants were cleared of cellular components by centrifugation (850 g, 5 min). Optical density (OD) quantification and calculations were performed with the Multiskan Ascent Type 354, 200-240V plate reader using a 4 parameter logistic.

Tumor cell proliferation was investigated by cell counting using the Coulter Counter Z2 from Beckmann. This method is based on the principle that particles (cells resuspended in an electrolyte) passing through an electric field alter the electrical resistance. The cell number was analyzed according to the manufacturer's instructions. Briefly, the cell suspension (i.e. trypsinized tumor cells) was mixed with the counter solution (Isotone II®) in a defined volume (9.5 ml counter solution Isotone II® and 500 μl sample, dilution factor 20) and counted for three times in the Coulter Counter Z2.

### Example 14

### Enzymatic Stability Testing of Oligonucleotides

### 3′-Exonuclease Digestion

Oligonucleotides (1 OD) were dissolved in 98 μl 0.05 M Tris-HCl buffer, pH 8.5. Two μl (0.083 U) phosphodiesterase I (from Crotalus adamanteus venom, 42 U/mg, 1 mg/ml, dissolved in 110 mM Tris-HCl, 110 mM NaCl, 15 mM MgCl₂, 50% glycerol, pH 8.9) was added. The mixtures were incubated at 37° C. (Thermocycler, Hybaid, PCR Sprint).

The unmodified phosphodiester oligonucleotide was analysed at 0 min, 5 min, 10 min, 20 min, 30 min, 1 h, 3 h, 6 h, and 24 h. Unmodified thiophosphate oligonucleotide, 3′-PEGylated thiophosphate, 5′-PEGylated thiophosphate, and 3′-5′-PEGylated thiophosphate oligonucleotide were analyzed at 0 min, 10 min, 30 min, 1 h, 3 h, 6 h, 24 h, 48 h, and 72 h.

For analysis, a 10 μl aliquot was drawn, heated to 95° C. for 10 min to destroy the phosphodiesterase, and centrifuged (5 min, 16060 g, room temperature). Nine μl of the 10 μl aliquot were taken, diluted with 11 μl 0.04 M KH₂PO₄, 0.002% H₃PO₄, pH 3.6, and analyzed by HPLC using a Waters 600 Pump, Waters 600 Controller, and Waters 2487 Dual-Absorbance Detector (254 nm). Separation was performed with a Merck LiChrospher 100 Reversed-Phase C18 column (endcapped, pore size: 100 Å, particle size: 5 μm, 125×4 mm) with a flow-rate of 1 ml/min in a gradient of 0, 13.5 and 50% acetonitrile/100 and 86.5, 50% 0.04 M KH₂PO₄, 0.002% H₃PO₄, pH 3.6, respectively.

### 5′-Exonuclease Digestion

Oligonucleotides (1 OD) were dissolved in 50 μl 0.2 M triethylammonium acetate buffer, pH 6.5. Fifty μl (0.22 U) phosphodiesterase II (Type I-SA, from bovine spleen, dissolved in water (4.4 U/ml)) were added. The mixtures were incubated at 37° C. (Thermocycler, Hybaid, PCR Sprint). The unmodified phosphodiester oligonucleotide was analysed at 0 min, 5 min, 10 min, 20 min, 30 min, 1 h, 3 h, 6 h, and 24 h. Unmodified thiophosphate oligonucleotide, 3′-PEGylated thiophosphate, 5′-PEGylated thiophosphate, and 3′-5′-PEGylated thiophosphate oligonucleotide were analyzed at 0 min, 10 min, 30 min, 1 h, 3 h, 6 h, 24 h, 48 h, and 72 h.

For analysis, a 10 μl aliquot was drawn, heated to 95° C. for 10 min to destroy the phosphodiesterase, and centrifuged (5 min, 16060 g, room temperature). Nine μl of the 10 μl aliquot were taken, diluted with 11 μl 0.04 M KH2PO4, 0.002% H3PO4, pH 3.6, and analyzed by HPLC using a Waters 600 Pump, Waters 600 Controller, Waters 2487 Dual-Absorbance Detector (254 nm). Separation was performed with a Merck LiChrospher 100 Reversed-Phase C18 column (endcapped, pore size: 100 Å, particle size: 5 μm, 125×4 mm) with a flow-rate of 1 ml/min in a gradient of 0, 13.5 and 50% acetonitrile/100, 86.5 and 50% 0.04 M KH2PO4, 0.002% H₃PO₄, pH 3.6, respectively.

### Results:

Surprisingly the immunostimulators linked with at least one polymer showed increased nuclease stability over those not linked with polymers. Oligonucleotides linked with polymers at the 3′- and 5′-end were surprisingly superior over those linked with a polymer of the same size/weight as the two polymers linked at only the 3′-end or the 5′-end of the oligonucleotide.

### Example 15

### Oligonucleotide Sequences

The TGF-beta1, TGF-beta2 and TGF-beta3 oligonucleotides of this example linked with at least one polymer are also part of the invention. There are further embodiments for polymer-oligonucleotide conjugates inhibiting formation of TGF-beta 1, 2 and/or 3 “in vitro” and “in vivo” and at least one of these conjugates and compounds, respectively, or a pharmaceutical composition comprising at least one of such conjugate or compound is suitable for controlling, preventing, and/or treating of unwanted neoplasms, fibrosis or viral diseases or disorders such as HIV as described in this invention.

### TGF-beta1, -2 and -3 Antisense Oligonucleotides:

gtgccatcaatacctgcaaa, catcagttacatcgaaggag, tcttgggacacgcagcaagg,
gaaatcaatgtaaagtggac, catgaactggtccatatcga, gaggttctaaatcttgggac,
gcactctggcttttgggttc, tagctcaatccgttgttcag, ccctagatccctcttgaaat,
accaaggctctcttatgttt, tcgagtgtgctgcaggtaga, tgaacagcatcagttacatc,
gctgggttggagatgttaaa, agaggttctaaatcttggga, cgccggttggtctgttgtga,
ctgctttcaccaaattggaa, aagtatagatcaaggagagt, tgctcaggatctgcccgcgg,
gtgctgttgtagatggaaat, agggcggcatgtctattttg, taagcttattttaaatccca,
tagctgcatttgcaagactt, tctgttgtgactcaagtctg, aagcaataggccgcatccaa,
tcaatgtaaagtggacgtag, attttagctgcatttgcaag, tgtagatggaaatcacctcc,
ttaacactgatgaaccaagg, attgtaccctttgggttcgt, agatccctcttgaaatcaat,
tgtaaagtggacgtaggcag, ccattcgccttctgctcttg, tgttaaatctttggacttga,
gaagggcggcatgtctattt, gaccctgctgtgctgagtgt, gaactagtaccgccttttca,
cgatcctcttgcgcatgaac, ccggccaaaagggaagagat, aaagagacgagtggctatta,
aagtggaaatattaatacgg, agatcaaggagagttgtttg, agttgtttttaaaagtcaga,
tgtaacaactgggcagacag, ggtgttgtaacaactgggca, tacccacagagcacctggga,
gggatggcatcaaggtaccc, tcgtcatcatcattatcatc, aagggtgcctattgcatagc,
ctcactgttaactctaagag, gcaaagtatttggtctccac, caagttccttaagccatcca,
ttatcttaatgcagactttc, cttacaagaagcttccttag, actggtgagcttcagcttgc,
acttgagaatctgatatagc, aggttcctgtctttatggtg, gtgtatccatttccacccta,
cagcacagaagttggcattg, gcaaggagaagcagatgctt, agcaaggagaagcagatgct,
ttttccaagaattttagctg, ttcttgttacaagcatcatc, ttaaagaaggagcggttcgg,
ctgggctgaaatttatatat, gggcagacagctaggagttt, gtgtactcaccaaggtaccc,
cccagcactttgggaggccg, ggctcacgcctgtaatccca, tgaccgtgaactcactattt,
atagtggtgatggctataca, ttttggttacctgcaaatct, gaacactcaccctgctgtgc,
gaatggctctttaaacccta, gaagaaatggagttcagtgt, tttctcctggaagggagagg,
aaatgcaacgcgttcccaac, aatacgaaacttttgcaaag, actagtaattctcagagcgg,
aagaaactagtaattctcag, agtgcatgtttttaaaagga, cagtagtgcatgtttttaaa,
ctcagcacacagtagtgcat, agatgcaggagcaaaaaggt, caggtagacagactgagcgc,
gcctcgatcctcttgcgcat, gcggatggcctcgatcctct, ctcaggatctgcccgcggat,
gctccggatagtcttccggg, agatggaaatcacctccggg, gttgtagatggaaatcacct,
ctggtactgttgtagatgga, aggcggctgecctccggctt, aacctccttggcgtagtact,
attttataaacctccttggc, cggcatgtcgattttataaa, cgggatggcattttcggagg,
gtagggtctgtagaaagtgg, tgaagtagggtctgtagaaa, attctgaagtagggtctgta,
aagcggacgattctgaagta, cccaggttcctgtctttgtg, ggcagtgtaaacttatttta,
ccatcaatacctgcaaatct, aggtgccatcaatacctgca, agttttctgatcaccactgg,
ttatagttttctgatcacca, cctagtggactttatagttt, acattagcaggagatgtggg,
agggcaacaacattagcagg, actccagtctgtaggagggc, tcctgcacatttctaaagca,
cagcaattatcctgcacatt, atgtaaagagggcgaaggca, ctcttaaaatcaatgtaaag,
ccaagatccctcttaaaatc, cctttgggttcatggatcca, gcattgtaccctttgggttc,
gcacagaagttagcattgta, ctgaggactttggtgtgttg, tcctgggacacacagcaagg,
tttagctgcatttacaagac, caaggactttagctgcattt, gtcattgtcaccgtgatttt,
ccagttttaacaaacagaac, agatgccagttttaacaaac, gttcattatatagtaacaca,
atgaaaggttcattatatag, ttccaagggtaatgaaaggt, cttaagccatccatgagttt,
cctggcttatttgagttcaa, ttagtcctataacaactcac, gcaaagaaccatttacaatt,
cttgcttaaactggcaaaga, acatgtaaagtagttactgt, acacattacatgtaaagtag,
taagatctacacattacatg, attcaaaggtactggccagc, tttgtagtgcaagtcaaaat,
catgtcattaaatggacaat, cctacatttgtgcgaacttc, ttccccctttgaaaaactca,
tttttaatcagcctgcaaag, actgggcagacagtttcgga, taacaactgggcagacagtt,
tgttgtaacaactgggcaga, cacagagcacctgggactgt, gtacccacagagcacctggg,
tcaaggtacccacagagcac, tggcatcaaggtacccacag, ggcgggatggcatcaaggta,
tttgcaggtattgatggcac, ctccttcgatgtaactgatg, ccttgctgcgtgtcccaaga,
tcgatatggaccagttcatg, gtcccaagatttagaacctc, gaacccaaaagccagagtgc,
atttcaagagggatctaggg, aaacataagagagccttggt, tctacctgcagcacactcga,
tcacaacagaccaaccggcg, ttccaatttggtgaaagcag, actctccttgatctatactt,
ccgcgggcagatcctgagca, caaaatagacatgccgccct, tgggatttaaaataagctta,
cagacttgagtcacaacaga, ttggatgcggcctattgctt, ctacgtccactttacattga,
ggaggtgatttccatctaca, ccttggttcatcagtgttaa, acgaacccaaagggtacaat,
attgatttcaagagggatct, ctgcctacgtccactttaca, caagagcagaaggcgaatgg,
tcaagtccaaagatttaaca, aaatagacatgccgcccttc, acactcagcacagcagggtc,
tgaaaaggcggtactagttc, gttcatgcgcaagaggatcg, atctcttcccttttggccgg,
taatagccactcgtctcttt, ccgtattaatatttccactt, caaacaactctccttgatct,
ctgtctgcccagttgttaca, tgcccagttgttacaacacc, tcccaggtgctctgtgggta,
gatgataatgatgatgacga, gctatgcaataggcaccctt, ctcttagagttaacagtgag,
gtggagaccaaatactttgc, gaaagtctgcattaagataa, ctaaggaagcttcttgtaag,
gcaagctgaagctcaccagt, tagggtggaaatggatacac, caatgccaacttctgtgctg,
aagcatctgcttctccttgc, cagctaaaattcttggaaaa, gatgatgcttgtaacaagaa,
aaactcctagctgtctgccc, gggtaccttggtgagtacac, cggcctcccaaagtgctggg,
tgggattacaggcgtgagcc, tgtatagccatcaccactat, acactgaactccatttcttc,
cctctcccttccaggagaaa, gttgggaacgcgttgcattt, ccgctctgagaattactagt,
ctgagaattactagtttctt, tttaaaaacatgcactactg, atgcactactgtgtgctgag,
gcgctcagtctgtctacctg, atgcgcaagaggatcgaggc, agaggatcgaggccatccgc,
atccgcgggcagatcctgag, cccggaagactatccggagc, cccggaggtgatttccatct,
aggtgatttccatctacaac, tccatctacaacagtaccag, aagccggagggcagccgcct,
agtactacgccaaggaggtt, gccaaggaggtttataaaat, tttataaaatcgacatgccg,
cctccgaaaatgccatcccg, ccactttctacagaccctac, tttctacagaccctacttca,
tacagaccctacttcagaat, tacttcagaatcgtccgctt, cacaaagacaggaacctggg,
taaaataagtttacactgcc, tgcaggtattgatggcacct, ccagtggtgatcagaaaact,
tggtgatcagaaaactataa, cctgctaatgttgttgccct, gccctcctacagactggagt,
tgctttagaaatgtgcagga, tgccttcgccctctttacat, gattttaagagggatcttgg,
tggatccatgaacccaaagg, gaacccaaagggtacaatgc, tacaatgctaacttctgtgc,
ccttgctgtgtgtcccagga, gtcttgtaaatgcagctaaa, aaatgcagctaaagtccttg,
aaaatcacggtgacaatgac, gttctgtttgttaaaactgg, gtttgttaaaactggcatct,
tgtgttactatataatgaac, acctttcattacccttggaa, aaactcatggatggcttaag,
aattgtaaatggttctttgc, tctttgccagtttaagcaag, acagtaactactttacatgt,
ctactttacatgtaatgtgt, catgtaatgtgtagatctta, gctggccagtacctttgaat,
ctttgcaggctgattaaaaa, aactgtctgcccagttgtta, tctgcccagttgttacaaca,
acagtcccaggtgctctgtg, cccaggtgctctgtgggtac, gtgctctgtgggtaccttga,
ctgtgggtaccttgatgcca, taccttgatgccatcccgcc, ttccaccattagcacgcggg,
ccgtgaccagatgcaggatc

### Example 16

### Further Oligonucleotide Sequences

The oligonucleotides of this example linked with at least one polymer are further embodiments for polymer-oligonucleotide conjugates and compounds, respectively, or pharmaceutical compositions comprising or consisting of at least one of these conjugates or compounds in pharmaceutical acceptable carriers suitable for controlling, preventing and/or treating of unwanted neoplasms, fibrosis or viral diseases or disorders such as HIV.

cccggagggcggcatggggga, cctcagggagaagggcgc, gtaggagggcctcgaggg,
ctgcaggggctgggggtc, agggctggtgtggtgggg, ggcatgggggaggcggcg,
ccggagggcggcatgggg, ggggggctggcgagccgc, ggacaggatctggccgcggatgg,
ccccctggctcggggggc, gggccgggcggcacctcc, gggcagcgggccgggcgg,
acggcctcgggcagcggg, gggtgctgttgtacaggg, gggtttccaccattagcacgcggg,
tcatagatttcgtt, ttgtcatagattt, aagaacatatatatg, aagaacatatatat,
ttgaagaacatatata, ccgggagagcaacacggg, acttttaacttga, attgttgctgtattt,
attgttgctgtatt, aattgttgctgtatt, aattgttgctgtat,
ggcgagtcgctgggtgccagcagccgg, ggcgagtcgctggg, acatcaaaagataa,
tgacatcaaaagat, gggccctctccagcgggg, gggctcggcggtgccggg, ggggcagggcccgaggca,
ggctccaaatgtaggggc, cgggttatgctggttgtacagggc, cggcgccgccgaggcgcccggg,
ggggcggggcgggacc, gggcggggcggggcgggg, gggcggggtggggccggg,
gggcaaggcagcgggggcgggg, cggtagcagcagcg, ccagtagccacagc, gcaggtggatagtcc,
cttgcaggtggatag, cgatagtcttgcagg, ccatgtcgatagtcttgc, ctcgatgcgcttccg,
cctcgatgcgcttcc, ggatggcctcgatgc, ggacaggatctggcc, cgcagcttggacagg,
gagccgcagcttgg, cgagccgcagcttg, acctccccctggct, ccaccattagcacg,
gaacttgtcatagatttc, gctgtgtgtactctgc, gctccacgtgctgc, gaattgttgctgtatttc,
gccaggaattgttgc, gtgacatcaaaagataac, ggctcaaccactgcc, gctgtcacaggagc,
cctgctgtcacagg, gcagtgtgttatccctgc, gcagtgtgttatccc, ccaggtcacctcgg,
gccatgaatggtggc, gccatgaatggtgg, ccatgagaagcagg, ggaagtcaatgtacagc,
ccacgtagtacacgatgg, gcacttgcaggagc, ccatggcagtgacc, ggctcctccatggc,
gctaggatctgactgc, cctgactcagaggg, ggtctgaaaatgtttcc, ccattgcttgggacgg,
gcatcaaatcatcc, ccattgttcaatatcg, ggtcttcagtgaacc, ggagcttcatctggacc,
cctctggcattctgg, agggacagaagatg, gttttctgggaagg, ggttttctgggaag,
aggttttctgggaag, gtaggttttctggg, ggtaggttttctgg, ccagaatgcaagaagcc,
gctgtcccagaatgc, gcaagtcacagacttggc, ccacagctgcacagg, ggtgtggaatcaacc,
gtcatgtgctgtga, cgctatctgagcagcg, ccagtgtgatgatgg, ccagtagattaccactgg,
ggcacaaacacgcacc, ccacggatctgaagg, cggaacatctcgaagcg, cctcattcagctctcgg,
ccttgagttccaagg, cctttttggacttcagg, ggaggtagactgaccc, aaaatgtttcct,
tgaaaatgtttc, ctgaaaatgttt, tctgaaaatgttt, tctgaaaatgtt, aaatcatccatt,
ttgttcaatatc, attgttcaatatc, attgttcaatat, cattgttcaatat, cattgttcaata,
aaaagtgtttct, acatgagttttttat, aacatgagttttttat, acatgagtttttta,
aacatgagtttttta, aacatgagtttttt, aaaacatcttgtt, cagagggggctcgacgc,
ctgactcagagggggctc, agggggacagaacg, ttgggacggcaagggggacagaa,
tgggacggcaaggggga, gccacggggggagca, gcaggggccacggggggag, aggggccacggggg,
caggggccacgggg, ggtgcaggggccacg, tggtgcaggggccgccgg, ggggctggtgcaggggcc,
agggggctggtgcagggg, gggctggtgcaggg, gagggggctggtgcag, aggagggggctggtg,
gggccaggagggggctgg, aggggccaggagggggct, ggggccaggagggg, caggggccaggaggg,
tctgggaagggacaga, tgagggcaggggagta, ttgagggcaggggag, cgggtgccgggcgggggtg,
cggacgcgggtgccgggcgggggt, cgggtgccgggcggg, ggacgcgggtgccgggcg,
tgggggcagcgcctcaca, ggtgggggcagcgcct, ccattttagtgcacatccgg,
ccattttagtgcacatcc, gctgttccattttagtgc, gtagtcgtgtagag, gtttgtagtcgtgtag,
gtttcaggagtttgtag, ccagctccgaagagg, cgtcgtcgtgatcacg, ggtaaaagtactgtcc,
ggctttgacaaagcc, cttgtgcagatcgtccag, cgtggttcatcttgtgc, cacgtggttcatcttgtg,
cctccttgaaggtgg, cgctccactttgatgcg, ccttgtcctccagg, ggtactcgacagcc,
ctgacgtgggtcatg, ccgttgctgacgtgg, catcctccgcctcc, gtttccatcctccg,
ggtgtttccatcctcc, ggtgtttccatcctc, gctcagcgcctcatc, ccttcttcatcatgctgc,
ccttcttcatcatgctg, ccttcttcatcatgc, gcgtccttcttcatcatgc, cctgctcactcagg,
cgcaggcttgagcg, gccagcttcagcagc, ggtggtgaccagcc, cctcggcgaactcc,
gcttgtgtaaatcc, ggttctgcttgtgtaaatcc, gctgctcaggttcgc, gaaggcgaccgtcg,
cgaaggcgaccgtc, gcaccgtctgtggc, cgtgtccatgtcgatgg, cgtgtccatgtcgatg,
gcgtgtccatgtcg, ccagcttgcgcttgc, cgctccagcttgcg, cgtgttctgactcttgag,
cgtgttctgactcttg, gctgttgacgtggc, cgactcagtacgcc, gccatgcccgactc,
cccttggaggtggc, ttttagtgcacat, tgttccattttagt, aaaaaaagtggaag,
tacaaaaaaaagtg, atacaaaaaaaagt, catacaaaaaaaagt, catacaaaaaaaag,
gaaaaaaaacatac, cagaaaaaaaacatac, cagaaaaaaaacat, ttcaatatgaatcg,
tattcaatatgaatcg, tattcaatatgaatc, tattcaatatgaat, tatattcaatatgaa,
ttatattcaatatga, tattatattcaatatga, ttatattcaatatg, tattatattcaatatg,
attatattcaatat, tattatattcaatat, atatattatattcaatat, aaatatattatattcaatat,
tattatattcaata, atatattatattcaata, caaatatattatattcaata, tatattatattcaat,
aatatattatattcaat, tatattatattcaa, caaatatattatattcaa, caaatatattatattca,
caaatatattatattc, cacaaatatattatattc, aaatatattatatt, caaatatattatatt,
caaatatattatat, cacaaatatattatat, cacaaatatattat, tacacaaatatattat,
tacacaaatatatta, taaatacacaaatatatt, aatacacaaatata, gttaaatacacaaata,
tgttaaatacacaa, tttagagactaagt, ataaactctttaga, taaaataaactctttag,
taaaataaactcttta, ttaaaataaactcttt, cttaaaataaactc, taaaaagaacaaaca,
taaaaagaacaaac, caataaaaagaacaa, tcaataaaaagaacaa, tcaataaaaagaac,
ttcaataaaaagaa, tagattcaataaaaaga, tggcgcgggcgggtagc, gggctggcgcgggcgggtag,
tcgggggctggcgcgggcggg, tgggtgcctggtcgcgcgttctcggg, agggtccctgcggggccg,
gggagggtccctgcgggg, gggagggtccctgcgg, tgggccgggtccgc, tcccgggggtgtag,
agtactgtcccgggggtgt, gggacacgttggggggtg, gccgggggccccccggtagc,
cgggcccagccgggggc, cgggcccagccggg, gggaggtggctccgggccgg,
agggcggcgcgtgtggga, gggtggccaccggcgaaggg, aggggcaggggacgt,
taaaggggcaggggacgt, agggggtgtccgtaaagggg, ggggacgcgaacgtgccgccg,
cggggaacaagcggcccgggg, ggccgtcgggggcg, gcggccgtcgggggc, aggggggtaggaggcggg,
gcgctgggggcgcc, ggccgtcggggggt, ggggaggccagcttc, ggccgccaccttgggg,
gcggccgccgccgggg, gggcgcggccgccgccgggg, ggggtggcggcggcgg, gggggtggcggcggc,
tggggcagcagctggcag, cggggcgcccacgacacc, cggggcgcccacgacac,
gggccgcaccctctccaagtccgggg, gcagcagtcagtgg, ccattgtctagcacgg,
ggtctccattgtctagc, ggtggtattgttcagc, gctggatcaagaccc, ccacaaaatcgtgtcc,
ccttccacaaaatcgtgtcc, ggttgttcttgtgg, cctcttggttgtgc, ccagagtctcaaacacttgg,
ggtaacctgtgatctcttcc, cctgcagtactcgg, ggcattcacatactcc, gcaaacagtgcctggc,
cgcatcgtgtacttccg, gcacgttccgagcg, ggtaccagatactcc, ccagtggagacctgg,
cctgaggacacatcagg, cctcacttggttgtgagc, ggaagatgtccttcc, gcacactgctcatggc,
gctgtcacctcttgg, cctctgctgtcacc, ccacacatcactctgg, cctcctcttcagagg,
ccttctggttcacactgg, catggtgctcactgcg, cttggttgtgagcg, ggacaggcagtcac,
gtcacctcttggttgtgc, ccagagtctcaaacac, cacatactccctgg, gaccagcacgttccg,
gttggtgtctatcagtg, ccctggtagaggtg, ctcaaacacttggagc, cacacatcactctggtgg,
gcacagacagtgcgc, catggcagcagtcag, ctgctcatggcagcag, catctggaaacttccagatg,
ctggaaacttccag, cataactccacacatcactc, caccataactccacacatc, ctggtgggtgaacc,
cggattacttgcagg, cgctaggtgtcagcg, gccatcacgtatgc, gcatacaccagttcagc,
ccatcaaatacatcgg, ccagcagaagtcagg, gcttcatgtctgtgc, ggtgagttccaggtttcc,
ccacaaaatcgtgtcctgg, cccttacacatcgg, gcagctcacagatgc, gcactggtaactgc,
cctggatattggcactgg, ccagcaaactcctgg, gcagaaatgccaggc, ccattgtgcagaattcg,
ccctgcagtactcgg, ggcattcacatactccc, ggtcaggtttcacacc, ccaggtccacacagg,
ccttgtcatccagg, ggatcccaaagacc, cctcaacactttgatgg, gctgtgtcaccagc,
ggtctaagaggcagcc, ggcaatctgcatacacc, cctgtgtacgagcc, ccatccacttgatgg,
cccacacagtcacacc, ccatcgtaaggtttgg, ccttttccagcagg, ggagaattcagacacc,
ccaagtcctcattctgg, ccatcagtctcagagg, cctttgaaggtgctgg, ggcatggcaggttcc,
cctggcatggcagg, agatgtataggtaa, attttcacattctc, aattttcacattctc,
aattttcacattct, gaattttcacattc, ggaattttcacatt, agatttctttgttg,
aagatttctttgttg, aagatttctttgtt, taagatttctttgtt, ctaagatttctttgtt,
taagatttctttgt, ctaagatttctttgt, ctaagatttctttg, tctaagatttcttt,
gtctaagatttcttt, gtctaagatttctt, ttcgtctaagattt, attttgacatggtt,
aattttgacatggtt, aattttgacatggt, taattttgacatggt, taattttgacatgg,
gtaattttgacatg, tgtaattttgacatg, tgtaattttgacat, tctgtaattttgacat,
ctgtaattttgaca, tctgtaattttgaca, tctgtaattttgac, gtctgtaattttga,
aagtctgtaattttga, agtctgtaattttg, aagtctgtaattttg, aagtctgtaatttt,
gaagtctgtaatttt, gaagtctgtaattt, atgtagacatcaat, atcatccaacattt,
aatcatccaacattt, aatcatccaacatt, accatcaaatacat, aaaaacgtctttga,
ttttgttcttagaca, ttttgttcttagac, taaacagaaaagca, actaaacagaaaag,
aaactaaacagaaaag, aactaaacagaaaa, aaactaaacagaaaa, aaactaaacagaaa,
taaaaactaaacagaaa, aaaactaaacagaa, gtaaaaactaaacagaa, aaaaactaaacaga,
taaaaactaaacaga, taaaaactaaacag, gtaaaaactaaaca, aaaaagtaaaaactaaaca,
agtaaaaactaaac, aaaaaaagtaaaaactaaac, aagtaaaaactaaa, aaaaaaagtaaaaactaaa,
aaagtaaaaactaa, aaaagtaaaaacta, aaaaaaagtaaaaacta, aaaaagtaaaaact,
aaaaaaagtaaaaact, aaaaaaagtaaaaac, caaaaaaagtaaaaac, aaaaaaagtaaaaa,
caaaaaaagtaaaa, aacaaaacaaaaaaagtaaa, aaacaaaaaaagta, caaaacaaaaaaagta,
caaaacaaaaaaagt, caaaacaaaaaaag, ctttaaaaaaacaaaac, tctttaaaaaaacaaa,
gtctttaaaaaaacaaa, gtctttaaaaaaaca, gtctttaaaaaaac, tttatttcgtcttt,
tctttatttcgtct, tatttgcaaatgga, tatatttgcaaatgg, tatatttgcaaatg,
caaaatatatttgcaaatg, caaaatatatttgcaaat, caaaatatatttgca, caaaatatatttgc,
ttccaaaatatatttg, ttttccaaaatatattt, gttttccaaaatatatt, gttttccaaaatat,
ggttaggcaaagcc, ccgagaacatcatcgtgg, ccgagaacatcatcgtg, ccgagaacatcatcg,
cgtagtctgcgttgaagc, ccatgctggagaagg, ccgtgcagaagtcc, ggaatgaagttggc,
tgaccgtgggaatg, tggcagtgaccgtg, agatggcagtgacc, cgagatggcagtgacc,
ccagccactgcagg, gcaccagccactgc, ccctggagtaagcc, ggagataactgttccacc,
ggagataactgttcc, cttctagttggtctg, catcttctagttgg, tctcatcttctagttgg,
ctgcaaagcagacttctc, ccttcagcaggttgg, cccaggtcatcagg, ccagtcagatcaagg,
ggtgaaggcctcctc, cagggtgaaggcctc, cctggatgatgctgg, ccactgtgcagagg,
ggagtacaggtgacc, gctcattgctgctgc, ggaaggctcattgctgc, ttttctcttcttct,
atcttattcctttc, catcttattccttt, tagtttttccttct, tctagtttttcctt,
aactctagtttttc, gaactctagttttt, tgaactctagttttt, atgaactctagttttt,
tgaactctagtttt, atgaactctagtttt, atgaactctagttt, gcacacagtagtgc,
gcaggatcagaaaagc, gcaggtagacaggc, gcttgctcaggatctgc, gcaagtccctggtgc,
cctggagcaagtcc, cgtagtactcttcgtcg, cgtagtactcttcg, gtaaacctccttgg,
gtctattttgtaaacctcc, gcatgtctattttgtaaacc, ggcatcaaggtaccc, ggcatcaaggtacc,
gctttcaccaaattggaagc, gagaatctgatatagctc, ggagatgttaaatctttgg,
gctgtcgatgtagc, ccaggttcctgtctttatgg, cagcagggacagtg, cttgcttctagttcttcac,
gccatcaatacctgc, ggtgccatcaatacc, ccactggtatatgtgg, ggactttatagttttctg,
ctcaagtctgtaggag, ggtctgttgtgactc, caattatcctgcacatttc, gcagcaattatcctgc,
ggcagcaattatcc, ggttcgtgtatccatttcc, gcacagaagttggc, ccagcacagaagttgg,
gtgctgagtgtctg, cctgctgtgctgagtg, gctcaggaccctgc, gcagcaaggagaagc,
ccaatgtagtagagaatgg, gctgcatttgcaag, aaaaaagaaatcaa, aaaaaaagaaatcaa,
aaaaaaaagaaatcaa, taaaaaaaagaaatcaa, ataaaaaaaagaaatcaa,
aataaaaaaaagaaatcaa, gaataaaaaaaagaaat, agaataaaaaaaagaaat, cagaataaaaaaaa,
tcagaataaaaaaa, ttgtttttaaaagt, agttgtttttaaaa, aagttgtttttaaaa,
aaagttgtttttaaaa, aaaagttgtttttaaaa, aaaaagttgtttttaaaa,
aaaaaagttgtttttaaaa, aaaaaaagttgtttttaaaa, aaaaaaaagttgtttttaaa,
tttttaaaaaagtg, ttttttaaaaaagtg, attttttaaaaaagtg, cattttttaaaaaagt,
gcattttttaaaaaa, tgcattttttaaaaaa, agcttattttaaat, aagcttattttaaat,
taagcttattttaaat, tgtaattattagat, atgtaattattagat, tgatgtaattatta,
atgatgtaattatta, atggtattatataa, tatggtattatataa, ttatggtattatataa,
tttatggtattatataa, atttatggtattatataa, aatcatattagaaa, ttacaatcatatta,
tttacaatcatatta, ggcatgacgcctttcc, gcatgacgcctttc, gcctgacgagaggc,
ctcaagcctgacgag, ccacagttcctttttc, gctgcaataaagatacag, gctgcaataaagatac,
ggacactgatttctatg, gcattatcaactttgg, acttttagcaccaatg, ccaagaaacttttagcacc,
ccagatcatcttcc, agtcaaggacacatag, tctttgagcaacatgg, gggtataacagctg,
gaggtgaaccattaatgg, tcttcgtatcgtttag, tgttggatagtgttc, gttgatcacttgctg,
ggattccattactcg, gacatatgaaaaatgttgtc, gccaataaagacatatg,
ccagaatcaagattctg, ctgttccagaatcaag, gacaaatctgttccagaatc,
ggaaagacaaatctgttcc, gattaagaggacaagc, ggaagattaagagg, gcagtgtgattattctgg,
ggagaaagatacatatctg, ggagatcttacagg, gcatttgcagtagaatttac, cagtgaaagagagg,
gctagccgatacac, ggaagatccttgtatgc, gcatgaggaagatcc, ggagtcatttttgttg,
ccaattgatactaagattc, tcttttgagcacacg, ccttcagcacttcttttg, ggttgcttccttcagc,
cagtggtttaggag, cctgagatcctcatttc, ccaaggtcctgagatcc, ggtgtacacagtgtcc,
tatctttaatttct, tcttttgaatataa, ttcttttgaatataa, tttcttttgaatataa,
ttttcttttgaatataa, tttttcttttgaatataa, atttctatgttttt, ttaaagaatttatg,
gttaaagaatttat, agttaaagaatttat, aagttaaagaatttat, taagttaaagaatttat,
tttagtaagttaaa, ttttagtaagttaaa, atttcttttagtaa, aatttcttttagtaa,
atcaatttctttta, tatcaatttctttta, aatatataagttca, aaatatataagttca,
caaatatataagtt, tcaaatatataagtt, tgtcaaatatataa, aatttatttcagta,
aataaaaatgtgat, taataaaaatgtgat, tagctaataaaaat, ttagctaataaaaat,
tttagctaataaaaat, aataaaatagtcaa, taataaaatagtcaa, ttaataaaatagtcaa,
tttaataaaatagtcaa, gtttaataaaatagt, agtttaataaaatagt, gagtttaataaaata,
agagtttaataaaata, aataattcttgtat, tatattacattcat, atctatattacatt,
ataaacatttttca, aataaacatttttca, aaataaacatttttca, gaaataaacattttt,
tgaaataaacattttt, ttgaaataaacattttt, tttgaaataaacattttt,
ttttgaaataaacattttt, tttttgaaataaacattttt, atttttgaaataaacatttt,
aatttttgaaataaacatt, aaatttttgaaataaacatt, aaaatttttgaaataaacat,
taaaatttttgaaataaaca, ataaaatttttgaaataaac, tataaaatttttgaaataaa,
gtataaaatttttgaaat, ggtataaaattttt, aggtataaaattttt, aaggtataaaattttt,
aaaggtataaaattttt, aaaaggtataaaattttt, taaaaggtataaaattttt,
ataaaaggtataaaattttt, tttagaaagatttt, aagataaatttctt, taagataaatttctt,
ttaagataaatttctt, tttaagataaatttctt, ttttaagataaatttctt,
tttttaagataaatttctt, atttttaagataaatttctt, tatttttaagataaatttct,
ttatttttaagataaatt, tttatttttaagataaatt, ctttatttttaagataaat,
tctttatttttaagataaat, atctttatttttaagataaa, atctttatttttaa,
gatctttatttttaa, agatctttatttttaa, tagatctttatttttaa, aatcatcattaatt,
aaatcatcattaatt, aaaatcatcattaatt, taaaatcatcattaatt, ttaaaatcatcattaatt,
tttaaaatcatcattaatt, atttaaaatcatcattaatt, aatttaaaatcatcattaa,
gaatttaaaatcat, tgaatttaaaatcat, ttaaaataggaaat, aatttctctttaaa,
aaatttctctttaaa, taaaattttgaatg, ctaaaattttgaat, tttgctaaaatttt,
atatgaaaaatgtt, ttttaaattaagca, ttgtaaaaatcaaa, tttgtaaaaatcaaa,
tttgataaaacttt, atgttttatcattt, aatgttttatcattt, aaatgttttatcattt,
taaatgttttatcattt, tctaaatgttttat, ttctaaatgttttat, taagatcaaataaa,
ataagatcaaataaa, aataagatcaaataaa, taataagatcaaataaa, ttaataagatcaaataaa,
tttaataagatcaaataaa, ttgtttaataagat, attgtttaataagat, tgattgtttaataa,
ttgattgtttaataa, tttgattgtttaataa, ttttataaaacagt, tttttataaaacagt,
ttttttataaaacagt, cttttttataaaaca, acttttttataaaaca, cacttttttataaaa,
acacttttttataaaa, tacacttttttataaaa, atacacttttttataaaa, attttgaatttaag,
gattttgaatttaa, tgattttgaatttaa, atgattttgaatttaa, aatgattttgaatttaa,
ataatagaatcata, tataatagaatcata, tataatagaatcat, tactataatagaat,
atactataatagaat, aatactataatagaat, agaatactataata, tagaatactataata,
atagaatactataata, tatagaatactataata, ttatagaatactataata, aatatttgttttca,
aaatatttgttttca, aaaatatttgttttca, caaaatatttgtttt, aaattttatatgga,
tgaaattttatatg, ctgaaattttatat, tctgaaattttatat, ttctgaaattttatat,
atctgatttatttt, aagatattaaatgt, tgaagatattaaat, ataaataacaatga,
tataaataacaatga, gtataaataacaat, tgtataaataacaat, ttgtataaataacaat,
tcttgtataaataa, atcttgtataaataa, aatcttgtataaataa, acaactttttaaat,
tacaactttttaaat, tacaactttttaaa, cggggggttttgggcggcatg,
ttttcggggggttttgggcggca, tcggggggttttgggcggc, ggtggcggccgtttttcggggggt,
ccgggggttccgcggcggcagcg, cgggggttccgcggcgg, ggcggcggtgccgggggttccgc,
ggagggggcggcggcggcggtg, gggggcggcggcggcgg, ggggcggcggcggcg,
agggggcctggtggaag, tagggggcctggtg, gtagggggcctggt,
gaggtattggtgacaaggtagggggc, tcttcaggggtgaaatatagatgttc, ggactcttcaggggtg,
tcggactatactgc, cagttcggactatact, aagcctaagacgca, gcccaagttcaaca,
tgaaaagtcgcggt, ggttaattaagatgcctc, tctctaagagcgca, acgtgaggttagtttg,
cacgtgaggttagt, catagaacagtccg, cagtcatagaacagtc, ctttgcagtcatagaaca,
tgcagtcatagaac, ggtcgtttccatct, catagaaggtcgtttc, cgtcatagaaggtc,
catcgtcatagaagg, ggacgggaggaacgaggcgttgag, tagccataaggtcc, ggttactgtagcca,
ggttactgtagcca, agttcttggcgcggaggt, aggtgaggaggtccgagt, tggactggattatcag,
gtggtggtgatgtgcccg, tgtcacgttcttgg, ctcatctgtcacgt, cgaagccctcggcgaacc,
gcgtgttctggctgtgcagttcgg, ctgccccgttgacc, aggtttgcgtagac, ggttgaagttgctg,
ctgggttgaagttg, tgctgcacgggcatctgctg, ggcactgtctgaggctcctccttcagg,
actccatgtcgatg, ctctccgccttgatcc, gttcctcatgcgcttc, ctgagctttcaagg,
gcgattctctccagcttcctttttcg, ctgagctttcaaggttttcactttttcctc, tccctgagcatgtt,
tctgtttaagctgtgc, ctttctgtttaagctgtg, ggttcatgactttctg, cgtggttcatgact,
actgttaacgtggttc, ccactgttaacgtg, cccactgttaacgt, agcatgagttggca,
gcgttagcatgagt, gtttgcaactgctg, caaaatgtttgcaactgc, tccattttagtgcacatc,
ctgttccattttagtgca, gtgtatgagtcgtc, ctgtgtatgagtcg, cgtagctgtgtatg,
tcgtgtagagagag, agtttgtagtcgtgtaga, gtttgtagtcgtgtag, agtttgtagtcgtg,
ggagtttgtagtcg, tcaggagtttgtagtc, gtttcaggagtttgtagt, tcggtttcaggagt,
ttgagactccggta, accagaaaagtagctg, cctgaccagaaaag, attcaggcgttcca,
ggtaaaagtactgtcc, gggtaaaagtactgtc, gcacctccaccgctgcca, ctcctgctcctcggtgac,
gctttgacaaagcc, cttgtgcagatcgt, tcatcttgtgcagatc, gttcatcttgtgcaga,
cgtggttcatcttg, tcacgtggttcatc, ggttggtgtaaacg, tacgagctcccggtcccgac,
tagctgatggtggt, tccttgaaggtgga, tcttccatgttgatgg, ctttgatgcgctct,
ctccactttgatgc, gctccagcttccgcttccggcacttggtgg,
ggccttgagcgtcttcaccttgtcctccag, tgaccttctgtttgag, catgaccttctgtttg,
gtcatgaccttctg, cgagaacatcatcg, gtagtctgcgttga, gctgcagcgggaggatgacg,
agtaagagaggctatc, gtagtaagagaggc, ggtagtaagagagg, gtgagtggtagtaaga,
gtccgtgcagaagtcctg, gaatgaagttggcact, ggaatgaagttggc, gggaatgaagttgg,
gctgcaccagccactgcaggtccggactgg, tcatggtcttcacaac,
caatgctctgcgctcggcctcctgtcatgg, ctagagttcctcac, gagtacgctagagt,
gaagagtacgctag, ctgcttcccacccagcccccacattccc, ttcatcctctgtactgggct,
gttacggatgtgca, cagttacggatgtg, ccagttacggatgt, agagtctgagttgg,
gtgagactcagagt, tcttagggtgagac, gagagtacttcttagg, ggaagaaactatgagagt,
cttagggaagaaactatg, cggtaagaaacttagg, agcatgcggtaaga, gtctgaaagcatgc,
agaacaaagaagagcc, caagagaacaaagaagag, cagcaagagaacaaag, tcctcagcaagaga,
aggtgtgacttgca, gaataggtgtgacttg, cagaataggtgtgact, gcagaataggtgtg,
cagttgcagaataggt, gaaaccatttctgacc, tgtgaaaccatttctgac, cactgtgaaaccatttct,
ccactgtgaaacca, agaactggctcctgcagcttccctgcttcc, cacctccattcaccc,
cagtaaaagtgtctgc, cgacattcagtaaaagtg, gaccgacattcagt, cttctggagataactaga,
catcttattcctttccct, cagccatcttattcct, tgcagccatcttattc, gagtgtatcagtcag,
ggagtgtatcagtc, cttggagtgtatcagt, acagagtacctacc, ccaactttcccttaag,
ccttatgctcaatctc, gtcttactcaaggg, acagtcttactcaagg, cataagacacagtcttac,
gaaagcataagacacagt, ggaaagcataagacac, agggataaaggaaagc, cctgtatacagagg,
tgtctcctgtatacag, catcttctagttggtc, ctcatcttctagttgg, cttctcatcttctagttg,
caaagcagacttctca, ctgcaaagcagact, ctagtttttccttctcct, tctagtttttccttctcc,
caggatgaactctagt, tcgtagaaggtcgt, agggttactgtagc, gtagtggtgatgtg,
cgtcgtagaaggtc, tttcgtgcacatcc, agtttgtagtcgtgaaga, cgagaacatcatgg,
gtagtaggaaaggc, ggtagtaggaaagg, ggaatggtagtagg, ggtcattgagaagag,
gctaatgttcttgacc, gccaaggtcctcat, ggagtctatctcca, ccaaagaatcctgact,
cacatgcttagtgg, ctcgtaaatgaccg, aggaatctcgtaaatgac, cagcagcgattcat,
ggagatcatcaaagga, ctcagcaatggtca, gatctcgaacacct, cacaatctcgatctttct,
ccttcttaaagattggct, cacataccaactgg, agcttgatgtgagg, gaagttgtagcttgatgt,
gcttgaagttgtagct, ctgcttgaagttgtag, gacacaactcctct, tcctttgatagacacaac,
ctcgtttgatagacac, ggttagcacacact, ggtaacggttagca, cgtaacacatttagaagc,
ctcatccgtaacac, ccggtaagtattgtagtt, ggtgtatttccttgac, acataccaactggtgt,
gtccctatacgaac, ttcatgtctgtgcc, gtaggtgagttcca, gttgtgagcgatga,
catagttgtcctcaaaga, ggcatagttgtcct, cattgtctagcacg, ctccattgtctagc,
gtattgttcagcgg, tcaagatctctgtgag, cacaaaatcgtgtcct, tccttccacaaaatcg,
gtggaagatgtcct, tcttgtggaagatgtc, tctatcagtgtgagag, ggttggtgtctatc,
acatcggagaacag, ccttacacatcgga, acaatcctcagaactc, gctctgacaatcct,
tggttgaagtggag, ctgtggttgaagtg, gttgtaggtgacca, ctgtgttgtaggtg,
gactcaaacgtgtc, catggactcaaacg, cgaatgtataccgg, ccgaatgtataccg,
gccgaatgtatacc, gtagttgtagggac, tagaaaggtagttgtagg, gtagaaaggtagttgtag,
cgtagaaaggtagttg, ccgtagaaaggtag, gaccatagcacact, ggatattggcactg,
cctggatattggca, gctcccaaagatct, cccatcaaagctct, caaacacttggagc,
gtctcaaacacttgga, gagtctcaaacacttg, gtaacctgtgatctct, ggtaacctgtgatc,
gtataggtaacctgtg, tgagatgtataggtaacc, tgctgagatgtatagg, ccatgctgagatgt,
ggattacttgcagg, tgttatggtggatgag, ggtgttatggtgga, gcagttgacacact,
agtactcggcattc, cattcacatactccct, tccaaaacaggtcact, ggtccttatagtgg,
cagaatgccaacca, acgagaatgccaac, gatcccaaagacca, tcgcttgatgagga,
catcgtgtacttcc, gcatcgtgtacttc, actgtgccaaaagc, cttgtagactgtgc,
cccttgtagactgt, tcaacactttgatggc, ccctcaacactttg, gtgttttccctcaaca,
gtatgcttcgtctaag, cgtatgcttcgtct, ccatcacgtatgct, gcataagctgtgtc,
catggtctaagagg, caatctgcatacacca, ggcaatctgcatac, ctgtctcgtcaatg,
cataactccacacatc, agtcacaccataactc, acagtcacaccataac, ccccaaaagtcatc,
tcgtaaggtttggc, gatcccatcgtaag, caatggtgcagatg, gacatcaatggtgc,
gtagacatcaatggtg, catgatcatgtagacatc, ccatgatcatgtagac, catttgaccatgatcatg,
ccaacatttgaccatg, tcatccaacatttgacca, gagtcaatcatccaacat, cagagtcaatcatcca,
ccgacattcagagt, gaattcagacaccaac, gatgaccacaaagc, ccatcaaatacatcgg,
tcaccatcaaatacatcg, caacgtagccatca, acgtctttgacgac, caaaaacgtctttgacga,
ggcaaaaacgtctttg, caaaggcaaaaacgtc, gtgtcaagtactcg, gtaatagaggttgtcg,
cccagtaatagagg, catggtgctcactg, gtgcctgtacgtac, tgcaggtggatagt,
catgtcgatagtcttgca, gtcgatagtcttgc, ccatgtcgatagtc, ctccatgtcgatag,
cttggacaggatct, tgctgttgtacagg, gtgctgttgtacag, ttggcgtagtagtc,
tccaccattagcac, gatttcgttgtggg, gtcatagatttcgttgtg, tgtactctgcttgaac,
gtgtactctgcttg, tgctgtgtgtactc, ctgatgtgttgaagaaca, ctctgatgtgttgaag,
gctctgatgtgttg, gagctctgatgtgt, cacttttaacttgagcct, ctccacttttaacttgag,
tgctgtatttctggtaca, ccaggaattgttgc, ttgctgaggtatcg, gataaccactctgg,
caaaagataaccactctg, cggtgacatcaaaag, cctcaatttcccct, gttatccctgctgt,
gcagtgtgttatcc, gatgtccacttgca, tagtgaacccgttg, tgccatgaatggtg,
gttcatgccatgaatg, catgagaagcagga, gctttgcagatgct, gagctttgcagatg,
tagttggtgtccag, ctgaagcaatagttgg, agctgaagcaatagttgg, ggagctgaagcaat,
caatgtacagctgc, ggaagtcaatgtacag, cggaagtcaatgtac, gcggaagtcaatgt,
agttggcatggtag, gcagaagttggcat, ctccaaatgtaggg, accttgctgtactg,
tgctggttgtacag, ggttatgctggttg, gtagtacacgatgg, cgtagtacacgatg,
cacgtagtacacga, catgttggacagct, gcacgatcatgttg, cacacagtagtgca,
gatcagaaaagcgc, accgtgaccagatg, gtagacaggctgag, tatcgagtgtgctg,
ttgcgcatgaactg, ttgctcaggatctg, actggtgagcttca, gctcaggatagtct,
tgtagatggaaatcacct, tggtgctgttgtag, ttctcctggagcaa, tactcttcgtcgct,
cttggcgtagtact, cggcatgtctattttgta, cgggatggcatttt, ctgtagaaagtggg,
acaattctgaagtagggt, attgctgagacgtcaaat, tctccattgctgag, tcaccaaattggaagcat,
ctctgaactctgct, aacgaaagactctgaact, tgggttctgcaaac, ctggcttttgggtt,
gttgttcaggcact, tctgatatagctcaatcc, tctttggacttgagaatc, tgggttggagatgt,
tgctgtcgatgtag, acaactttgctgtcga, attcgccttctgct, gaaggagagccatt,
tcagttacatcgaagg, tgaagccattcatgaaca, tcctgtctttatggtg, aaatcccaggttcc,
ggacagtgtaagcttatt, gtacaaaagtgcagca, tagatggtacaaaagtgc,
cacttttatttgggatgatg, gcaaatcttgcttctagt, gtgccatcaatacc, ggtatatgtggagg,
tctgatcaccactg, tcctagtggactttatag, tttttcctagtggact, caataacattagcagg,
aagtctgtaggagg, tctgttgtgactcaag, gttggtctgttgtg, caaagcacgcttct,
tttctaaagcaataggcc, gcaattatcctgcaca, acgtaggcagcaat, atcaatgtaaagtggacg,
ctagatccctcttg, ccatttccacccta, tgggttcgtgtatc, tggcattgtaccct,
tccagcacagaagt, ataaatacgggcatgc, agtgtctgaactcc, tgtgctgagtgtct,
ataagctcaggacc, aggagaagcagatg, agcaaggagaagca, aatcttgggacacg,
tagagaatggttagaggt, gttttgccaatgtagtag, cttgggtgttttgc, gcaagactttacaatc,
gcatttgcaagactttac, tttagctgcatttgcaag, gccacttttccaag, ttggtcttgccact,
cagcacacagtagt, cgatagtcttgcag, ctttcaccaaattggaag, caccaaattggaagc,
tcaccaaattggaagc, ctctggcttttggg, cggcatgtctattttg, cactacagacgagc,
cgtgcactacagacg, ggaacagttcgtcc, gaacagttcgtccatg, ccagagtttcggttc,
ctaggactgggacag, cgcacttgtagcg, ctcgcacttgtagc, gcacttgtagc,
gcgcactgtccctg, ccagggagatgcgc, gccggtgaggagg, ccggtgaggaggg,
cggttcactcggc, gagtttcggttcactc, ggcacgattgtcaaag, caggcgtcaccccc,
gcaggcgtcaccc, ctccctcctaagc, ccctcctaagcgg, cgagtccgcgttcg,
catcttctgccattc, gtgttttcccaccag, ggttttggttcactag, gcatcttcacgtctcc,
cttcacgtctcctgtc, gtcaccgcgtagtc, caaataggcaaggtc, cttgcaaataggcaag,
tgcttgcaaatagg, ctgcttgcaaatagg, gcaggtggatattt, ctgctgttggcag,
cactagtttccaagt, gttttggttcactag, ctttgatttcaggatag, gcacttcttctttatct,
ccaagtcagatttcc, gtttccaagtcagatttc, ggttcactagtttcc, ggttttggttcactag,
ccgaaaaattgggca, ccgaaaaattggg, ctatccgaaaaattgg, gttgataatgtcatcag,
ctcatgttgataatgtc, ctgtcaccgcgtag, cgtctcctgtcaccg, cttcacgtctcctg,
gagaactttatcatgtc, gctatatgcaggg, ccagctgctatatgcagg, aggctaaattttgcct,
ggctaaattttgcc, ggctaaattttgccttc, gcaggctaaattttgcc, gagttacccaagcg,
cagagttacccaagcg, cagagttacccaag, acagagttacccaag, ggtgcaaaacagag,
ctaggtgcaaaacag, gagaactttatcatgtcc, gctagatgaatggc, gcaaacatggcaggc,
cagcaaacatggca, gcagcaaacatggc, agcagcaaacatgg, cagcagcaaacatg,
agcagcagcaaaca, cagcagcagcaaaca, cagcagcagcaaac, caccagcagcagca,
gcattgacgtcagc, gatgttgtcgtgctc, tgagatgttgtcgtgct, tgagatgttgtcgtg,
gccaatgagatgttg, ctgccaatgagatg, cacatgggcatcac, tgtccacatgggca,
gtactgtccacatg, cagctgctatatgc, gttctccaccaggg, agttctccaccagg,
caaagttctccaccag, ccaagagtcatccagg, cccaagagtcatcc, cctgcattttcccaag,
tcctgcattttccc, gccatatctagaggc, tcacatcttcagcc, gcttcacatcttcagc,
cagcttcacatcttc, gtaacttatacagctgc, ccagtttttgtctgg, ccatttgtctcagg,
gtgtagcccatttg, gcttcggtgtagcc, gatcacttcaattgcttc, cttgtggaggcagg,
gctgccttgtggag, ctatttgctgccttgtgg, ggatgtctccacgc, ggaaggatgtctcc,
tgcggaaggatgtc, gtttgcggaaggatgtc, gctgagtttgcgga, ggtaaagctgagtttg,
tcggtaaagctgag, gactcggtaaagctg, agagactcggtaaagc, gaaattgtcagcaggc,
gaaattgtcagcagg, ggaaattgtcagcagg, ggaaattgtcagcag, gggaaattgtcagc,
gtgtgggaaattgtc, ggtttacacggtgtg, gctttggtttacacg, gcacctttgggatgc,
ccaggttctgcttcc, gctctgtctagtggc, actctccatgtctc, caactctccatgtctc,
caactctccatgtc, agcaactctccatg, gtagcaactctccatg, gtagcaactctcca,
ggttgtagcaactctcc, cgggcagtcctcca, gcaccgggcagtc, aggcaccgggcag,
gtgtgttaccaggtc, tgtgtgttaccaggt, tgggtcactgtgtg, cagactgtgggcatg,
cccaccagactgtggg, ccaccagactgtgg, tgcccaccagactg, cggcttcctcccc,
ccttgtcttccacc, accgaggctgccac, ggaagaaaccgagg, gggaagaaaccgag,
ggccatctgcgcc, gcggccatctgcg, gtggcggccatctg, accgtggcggccat,
gccgctcaatcttcatc, cttcatcttgtgatagg, gctcaatcttcatcttg, cagaaacactgttacag,
cagttgcagaaacactg, gtttcagttgcagaaac, cttccaccagaggg, gtcttccaccagag,
cttgtcttccaccagag, tccttgtcttccac, cttccttgtcttccac, catcttgtgataggg,
gctaggtgcagtggt, gatggctaggtgca, gtggatgatggctag, cccgtggatgatgg,
ctgcccgtggatga, agagcctccaccca, gttgtactctcgagc, cgttgtactctcg,
cgcgttgtactctc, gagtctccatgccg, ctgagtctccatgc, catggctgagtctc,
tgcatggctgagtc, gcgttcacgttggc, gtgcgagcgttcac, aggtgcgagcgttc,
gcaaaggtgcgagc, cctggtggctcagg, gtcagtcacctgag, caggtcagtcacctg,
cagcaggtcagtcac, gcagcaggtcagtc, catttagcagcaaggtc, gcagcatttagcagc,
ctgagcagcatttag, cccatgagaatcct, ccttcccatgagaatcc, tcctccccttccca,
gcctccagtagacc, gtcagacagggcct, ccatgtcagacagg, ggcccatgtcagac,
gctattcctgaaatcac, cctcttgtcttcttacc, ggagaagaaacctcttg, ccttgctgaagtttctt,
ccaagactccttgc, ccctttcatggagc, cctcttggtgtgac, gactaaggatgccg,
gtggcaggactaagg, agacgtggcaggac, cttccagcaggcag, gttcctctgcctgg,
gatgttcctctgcctg, gagatgttcctctgcc, gtgagatgttcctctg, cagagagtgagatgttcc,
ccagagagtgagatgttc, ggtccagagagtgag, gaggtccagagagtg, ggtcctgtagtgcc,
gattttatgatgcagge, gacctgcatcccttattg, tagttgattttccagcag,
gaatctcacgttttgc, cagagaaagaatctcacg, tttcaccatcagagaaag,
catttggacatttcacc, ccttcatttggacatttc, caatgtgcttgatgatcc, cgcatcggatttctc,
caaaccgcatcggatttc, gaactgcaaaccgc, gcagagaagaactgc, gcaagtaaacatggg,
ggtccacgttttgg, gcaagggtccacgttt, tggcttcttcttcaggg, tcctgctggcttcttc,
gtcctgctggcttc, ggtagtctaggaattgg, cttgcaggtagtctagg, gaaactcttgcaggtag,
caccaagaaactcttgc, cattacaccaagaaactc, ctcggtgttcattacacc,
ctttctattatccactcg, ccagtttagtctcaactt, aaccagtttagtctcaac,
acaaaccagtttagtctc, ctcgcgaaaaagtttctt, ccctcgcgaaaaagtttc,
gtccctcgcgaaaaag, cagttgaaccgtccc, gctttcgaagtttcagtt, gatgctttcgaagtttc,
ctgtctctgcaaataatg, cacttattacattcaccc, ttttcctccagttcctc,
ggacaatatgtacaaaactc, gttgatgaacatttggac, gtgttgatgaacatttgg,
caaaatttggccaggg, gcccaaaatttggcc, cccagcccaaaatttgg, gtccccagcccaaaatt,
aaatcgccagaggctg, accaaatcgccagagg, catcaccaaatcgccag, taggagtggttgaggc,
gtgtaggagtggttgag, ctgtgtaggagtgg, cccacatgcctgtg, cgatgaacaacgag,
ctggcgatgaacaacg, cgctggcgatgaac, gagctagtcccgttg, gcgaagagctagtcc,
ccagttatgcgaagagc, ccccagttatgcgaag, cacatgcttggcgc, gatcacatgcttggcg,
gacaaagagcatgatcac, gagtcacagggacaaag, gagagtcacagggac, gcagagagtcacagg,
ccatgcagagagtc, ccaccatgcagagag, tagccacgaccacc, gattagctgcccatcctt,
ggtatagattagctgcc, gtatcttctgtgaatggg, ctggcccacagtct, ctctggcccacagt,
tgcagggctctctg, agtgcagggctctc, cactgatcatgatggc, gacactgatcatgatggc,
acaatgacactgatcatg, gaaccaccaggaggat, gacacaaaacagccact, gtggacctttcggac,
caaccagcatacgaagt, tccctctgggcttc, actgtccctctggg, gactgtccctctgg,
cctagatgactgtccc, cagcgaggatactgc, cttcaccagcgaggat, tttcctctgggtcttcac,
ctttcctctgggtcttc, ctcccaatccaagtttt, ttcatcccggagcc, ttcttcatcccggagc,
gctcagccagttcttc, gacagagagggcac, cttcacctccgacag, gaaaagtctgggcagg,
gaccctggaacagaaaag, ctgaccctggaacag, actacaggctgaccct, attcactacaggctgacc,
cgattcactacagg, ggccgattcactac, cgaacgtctgttggtc, cgcgaacgtctgttg,
cttctgtttgtcgaggat, ttcaccaccttctgtttg, aggatgcgcttttcattc,
agcttgcaggatgcg, gttgacagcttgcaggat, ggaacggaaagttgacag,
aactcgagtttgacgagg, tgtccttgaaggagaac, cgtactccatgaccatgt,
gcacgtactccatgac, gattctccggcttcag, tcaatgagcagattctcc, ggtcaatgagcagattc,
ccctgctggtcaatg, tagccctgctggtc, cgcttggcgaaacc, ccttcacgcgcttg,
aaggtccaagtgcg, tgccgcacaaggtc, ggtgaggaccaccattt, gggtgtcacaggtg,
ataccatcttcttcaggg, ggtgataccatcttcttc, ccaggtgataccatcttc,
cctcactgctctggt, taagacctcactgc, cagagcctaagacctc, ccagagcctaagacc,
tcttcctttttgtgaagc, gaccaaattccatcttcc, atcagtggaccaaattcc,
ggttctttctggtccttt, tttttgggttctttctgg, ggtcttatttttgggttc,
aatgggcagactctcct, tccaccatgacctcaatg, aacggcatccaccatg, gtgaacggcatccac,
acttgagcttgtgaacgg, ttcatacttgagcttgtg, ctggtgtagttttcatac,
agctgctggtgtagtttt, aggaggaccagggt, aggtggtccaggag, tttctggccaaactgagg,
ggaggtttctggcc, tctggagtggccac, cttctggagcatgttgct, gccttctggagcatg,
gtttgtctggccttctg, gagtttgtctggccttct, ctagagtttgtctggcct,
gcaagggtaaaattctag, agtgcaagggtaaaattc, aaacaggcctccact,
cttggttaattccaatgg, aggcaactcccattagtt, tactactaaggcacaggg,
aatactactaaggcacag, gtacatcttcaagtcttc, ggagtggacatgat, aagaagatgaagcctttg,
ccgtcttactcttcttgg, ccgatacaattccaagg, ccttttccttctgag, ctgttgcaagtacg,
cagaagcagagggc, cctcagaagcagagg, ctcctcagaagcag, acaggctggtggca,
ccactctcaaacaggc, acggtagccgaagc, gacggtagccgaagc, ggccagacggtagc,
gtgtagggccagacggta, ccgaagccatttttcagg, ccccgaagccatttttc, ggttgatgtcgtcc,
gcttgagacactcgc, ccggacccgtccat, gcttgctttactgc, ggttgctctgagac,
gccacagtcatgcc, cgggcatgctggcg, gtgaagttcaggatgatc, ccagtgcctcatgg,
cagtgttctccatgg, ctgtaccagaccgag, gcatactgtttcagc, gccatcagctccttg,
ccacaccatagatgg, gctggagcagtttcc, ctcgcttctgctgc, accgtggcaaagcg,
aggtgacaccgtgg, gacttgattccttcag, ggatttgacttgattcc, gctgctgttcatgg,
ccgtttctttcagtagg, cttgaagtaggagc, cgctcctacatggc, gatgaggtacaggcc,
gtagatgaggtacag, gagtagatgaggtac, cctgggagtagatg, ggacctgggagtag,
acatgggtggaggg, gtgctcatggtgtc, ctttcagtgctcatg, tgctttcagtgctca,
gatgatctgactgcc, gttcgagaagatgatc, gggttcgagaagatg, ggtttgctacaacatg,
cagcttgagggtttg, tgcccctcagcttg, gacacacactatctc, gcagccatctttattc,
gttcagcagccatc, tggttcagcagcca, ctactggttcagcagc, tctactggttcagc,
gccacaaagttgatgc, cattgccacaaagttg, gagaacttggtcattc, ggtcaatgaagagaac,
cgatttccttggtc, ccgatttccttggtc, caaatagaggccgatttc, caaatagaggccga,
cctctaggctggct, catacctctaggctg, agccatacctctag, cagccatacctctag,
cacagagatagttacag, gtcttcgttttgaacag, ctagtcttcgttttgaac,
tagctagtcttcgttttg, gagccactgcgcc, cgtgagccactgcg, cgtaacgatcactgg,
gcactcgtaacgatc, ggagcactcgtaac, catcatcctgaggt, cagtatcatcatcctg,
ctcagtatcatcatcc, ctaaaagtatgtgccatc, cacatcgcctctct, gcttcacagtcacatcgc,
ggaaggcttcacagtc, cctgtgacttgagaattg, ggaagacctgtgac, ctctgctccacatatttg,
caacgaagatctctg, caacaccaacgaag, ggtcttctgtttgc, cgatgaagtggtaggaag,
ggttgcatggaagc, ggtcacaaacttgcc, ctgatttggtccactag, catgttagcactgttc,
ggtcttgatgtactcc, ccacctaaagagagatc, cttgtactgcaccatc, gccagttaagaagatg,
gagatcatgatccatgg, gtagtgtcccaatagtg, cttcctcatcattccc, cacaagcttttcgac

### Example 17

### TGF-Beta Protein

Presented are the amino acid sequences of TGF-beta1, TGF-beta2 and TGF-beta 3 with the international one letter abbreviation for amino acids.

RXXR: cleavage site of the mature (active) part (XX may be anything)
ASPC: the C of this motif is the C for the intermolecular cystine bridge that links the two monomers into a functional dimer
C C C: intramolecular cysteine bridges (cysteine knot motif) mature protein of TGF-beta 1, 2 and 3 contains 112 amino acids from the end of this listing

```
TGF-beta 1
MPPSGLRLLLLLLPLLWLLVLTPGRPAAGLSTCKTIDMELVKRKRIEAIRGQILSKLRLASPPSQGEVPPGPLPE
AVLALYNSTRDRVAGESAEPEPEPEADYYAKEVTRVLMVETHNEIYDKFKQSTHSIYMFFNTSELREAVPEPVLL
SRAELRLLRLKLKVEQHVELYQKYSNNSWRYLSNRLLAPSDSPEWLSFDVTGVVRQWLSRGGEIEGFRLSAHCSC
DSRDNTLQVDINGFTTGRRGDLATIHGMNRPFLLLMATPLERAQHLQSSRHRRALDTNYCFSSTEKNCCVRQLYI
DFRKDLGWKWIHEPKGYHANFCLGPCPYIWSLDTQYSKVLALYNQHNPGASAAPCCVPQALEPLPIVYYVGRKPK
VEQLSNMIVRSCKCS
preferred amino acid sequences of TGF-beta1:
  1) ALDTNYCFSSTEKNCCVRQL
  2) YIDFRKDLGWKWIHEPKGYH
  3) ANFCLGPCPYIWSLDTQYSK
  4) VLALYNQHNPGASAAPCCVP
  5) QALEPLPIVYYVGRKPKVEQ
  6) LSNMIVRSCKCS
  7) TEKNCCVRQLYIDFRKDLGW
  8) KWIHEPKGYHANFCLGPCPY
  9) WSLDTQYSKVLALYNQHNP
10) GASAAPCCVPQALEPLPIVY
11) YVGRKPKVEQLSNMIVRSCKCS
12) QYSKVLALYNQHNPGASAAPCCVPQALEPLPIVYYVGRKP
13) QYSKVLALYNQHNPGASAAPCCVPQALEPLPIVYYVGRKP
                                                I
        QYSKVLALYNQHNPGASAAPCCVPQALEPLPIVYYVGRKP
        (dimer of the TGF-beta1 amino acid sequence No. 12 coupled by an S-S
        bridge at the Cytosins of the AAPC motif)
14) ALDTNYCFSSTEKNCCVRQLYIDFRKDLGWKWIHEPKGYHANFCLGPCPYIWSLDTQYSKVLALYNQHNPGA
SAAPCCVPQALEPLPIVYYVGRKPKVEQLSNMIVRSCKCS
15) ALDTNYCFSSTEKNCCVRQLYIDFRKDLGW
16) KWIHEPKGYHANFCLGPCPYIWSLDTQYSK
17) VLALYNQHNPGASAAPCCVPQALEPLPIVY
18) YVGRKPKVEQLSNMIVRSCKCS
19) CVRQLYIDFRKDLGWKWIHEPKGYHANFCL
20) GPCPYIWSLDTQYSKVLALYNQHNPGASAA
21) PCCVPQALEPLPIVYYVGRKPKVEQLSNMI
TGF-beta 2
MHYCVLSAFLILHLVTVALSLSTCSTLDMDQFMRKRIEAIRGQILSKLKLTSPPEDYPEPEEVPPEVISIYNSTR
DLLQEKASRRAAACERERSDEEYYAKEVYKIDMPPFFPSENAIPPTFYRPYFRIVRFDVSAMEKNASNLVKAEFR
VFRLQNPKARVPEQRIELYQILKSKDLTSPTQRYIDSKVVKTRAEGEWLSFDVTDAVHEWLHHKDRNLGFKISLH
CPCCTFVPSNNYIIPNKSEELEARFAGIDGTSTYTSGDQKTIKSTRKKNSGKTPHLLLMLLPSYRLESQQTNRRK
RALDAAYCFRNVQDNCCLRPLYIDFKRDLGWKWIHEPKGYNANFCAGACPYLWSSDTQHSRVLSLYNTINPEASA
SPCCVSQDLEPLTILYYIGKTPKIEQLSNMIVKSCKCS
Preferred amino acid sequences of TGF-beta2
  1) ALDAAYCFRNVQDNCCLRPL
  2) YIDFKRDLGWKWIHEPKGYN
  3) ANFCAGACPYLWSSDTQHSR
  4) VLSLYNTINPEASASPCCVS
  5) QDLEPLTILYYIGKTPKIEQ
  6) LSNMIVKSCKCS
  7) VQDNCCLRPLYIDFKRDLGW
  8) KWIHEPKGYNANFCAGACPY
  9) LWSSDTQHSRVLSLYNTINP
10) EASASPCCVSQDLEPLTILY
11) YIGKTPKIEQLSNMIVKSCKCS
12) QHSRVLSLYNTINPEASASPCCVSQDLEPLTILYYIGKTPK
13) QHSRVLSLYNTINPEASASPCCVSQDLEPLTILYYIGKTPK
                                                I
        QHSRVLSLYNTINPEASASPCCVSQDLEPLTILYYIGKTPK
        (dimer of the TGF-beta2 amino acid sequence No. 12 coupled by an S-S
        bridge at the Cytosins of the ASPC motif)
14) ALDAAYCFRNVQDNCCLRPLYIDFKRDLGWKWIHEPKGYNANFCAGACPYLWSSDTQHSRVLSLYNTINPEA
SASPCCVSQDLEPLTILYYIGKTPKIEQLSNMIVKSCKCS
15) ALDAAYCFRNVQDNCCLRPLYIDFKRDLGW
16) KWIHEPKGYNANFCAGACPYLWSSDTQHSR
17) VLSLYNTINPEASASPCCVSQDLEPLTILY
18) YIGKTPKIEQLSNMIVKSCKCS
19) CLRPLYIDFKRDLGWKWIHEPKGYNANFCA
20) GACPYLWSSDTQHSRVLSLYNTINPEASAS
21) PCCVSQDLEPLTILYYIGKTPKIEQLSNMI
TGF-beta3
MKMHLQRALVVLALLNFATVSLSLSTCTTLDFGHIKKKRVEAIRGQILSKLRLTSPPEPTVMTHVPYQVLALYNS
TRELLEEMHGEREEGCTQENTESEYYAKEIHKFDMIQGLAEHNELAVCPKGITSKVFRFNVSSVEKNRTNLFRAE
FRVLRVPNPSSKRNEQRIELFQILRPDEHIAKQRYIGGKNLPTRGTAEWLSFDVTDTVREWLLRRESNLGLEISI
HCPCHTFQPNGDILENIHEVMEIKFKGVDNEDDHGRGDLGRLKKQKDHHNPHLILMMIPPHRLDNPGQGGQRKKR
ALDAAYCFRNVQDNCCLRPLYIDFKRDLGWKWIHEPKGYNANFCAGACPYLWSSDTQHSRVLSLYNTINPEASAS
PCCVSQDLEPLTILYYIGKTPKIEQLSNMIVKSCKCS
preferred amino acid sequences of TGF-beta3:
  1) ALDTNYCFRNLEENCCVRPL
  2) YIDFRQDLGWKWVHEPKGYY
  3) ANFCSGPCPYLRSADTTHST
  4) VLGLYNTLNPEASASPCCVP
  5) QDLEPLTILYYVGRTPKVEQ
  6) LSNMVVKSCKCS
  7 NLEENCCVRPLYIDFRQDLG
  8 WKWVHEPKGYYANFCSGPCP
  9) YLRSADTTHSTVLGLYNTLN
10) PEASASPCCVPQDLEPLTIL
11) YYVGRTPKVEQLSNMVVKSCKCS
12) THSTVLGLYNTLNPEASASPCCVPQDLEPLTILYYVGRTPK
13) THSTVLGLYNTLNPEASASPCCVPQDLEPLTILYYVGRTPK
                                                I
        THSTVLGLYNTLNPEASASPCCVPQDLEPLTILYYVGRTPK
        (dimer of the TGF-beta3 amino acid sequence No. 12 coupled by an S-S
        bridge at the cytosins of the ASPC motif)
14) ALDAAYCFRNVQDNCCLRPLYIDFKRDLGWKWIHEPKGYNANFCAGACPYLWSSDTQHSRVLSLYNTINPEA
        SASPCCVSQDLEPLTILYYIGKTPKIEQLSNMIVKSCKCS
15) ALDAAYCFRNVQDNCCLRPLYIDFKRDLGW
16) KWIHEPKGYNANFCAGACPYLWSSDTQHSR
17) VLSLYNTINPEASASPCCVSQDLEPLTILY
18) YIGKTPKIEQLSNMIVKSCKCS
19) CLRPLYIDFKRDLGWKWIHEPKGYNANFCA
20) GACPYLWSSDTQHSRVLSLYNTINPEASAS
21) PCCVSQDLEPLTILYYIGKTPKIEQLSNMI
```

### Example 18

### PEGylation of the N-Terminus of a Protein or a Peptide

The protein or peptide is synthesized according to Merrifield, wherein the functional groups of the side chains are protected by t-BOC, and the N-terminal and is protected by Fmoc. Fmoc is removed by incubation with 20° A) piperidine in DMF. After several washing steps the protein or peptide is incubated with PEG 400, -600, -800, -1000, -1500, -2000, -5000, -20000 or -50000-NHS ester for 24 h at 30° C. Afterwards, the protective t-BOC groups of the side chains of the amino acids are removed and the N-terminal PEGylated protein or peptide is cleaved from the support by incubation with hydrofluoric acid and purified via FPLC.

### Example 19

### PEGylation of the N- and C-Terminus of a Protein or a Peptide

The protein or peptide is synthesized according to Merrifield, wherein the functional groups of the side chains are protected by benzyloxy-carbonyl, and the N-terminal end is protected by Fmoc. After the synthesis of the protein or peptide, the protein or peptide is cleaved from the support, and purified. The C-terminal end is activated with dicyclohexylcarbodiimide (DCC) and incubated with PEG-200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000-amine (PEG-NH₂) for 15 h at 30° C. The C-terminal PEGylated protein or peptide is purified and the N-terminal protective group Fmoc is removed by incubation of the PEGylated protein or peptide with 20% piperidine in DMF. After several washing steps the protein or peptide is incubated with PEG 200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000-NHS ester for 24 h at 30° C. In the following, the C- and N-terminal PEGylated protein or peptide is purified on a FPLC and afterwards the side chains are deprotected by incubation in HBr/acetic acid. Finally, the conjugate is purified again.

### Example 20

### PEGylation of the C-Terminus and of Side Chains of a Protein or a Peptide

The protein or peptide is synthesized according to Merrifield, wherein the functional groups of the side chains are protected by an alloc protecting group, and the N-terminal end is protected by Fmoc. After the synthesis of the protein or peptide, the protein or peptide is cleaved from the support, the C-terminal end is activated and incubated with PEG-NH₂ as described in example 19. In the following the PEGylated protein or peptide is purified and the alloc protecting group of the side chains is removed by incubating the protein or peptide with tetrakis(triphenylphosphine)palladium along with a 37:2:1 mixture of chloroform, acetic acid, and N-methylmorpholine for 2 h. Afterwards the C-terminal PEGylated protein or peptide is washed with 0.5% DIPEA in DMF, 3×10 ml of 0.5% sodium diethylthiocarbamate in DMF, and then 5×10 ml of 1:1 DCM:DMF. In the following the protein or peptide is incubated with PEG 200, 300, 400, 500, 600, 700, 800, 900, 1000, 1250, 1500, 1750, 2000, 2250, 2500, 2750, 3000, 3250, 3500, 3750, 4000, 4250, 4500 or 4750, 5000, 10000, 20000, 50000 or 1000000-NHS ester for 15 h at 35° C. Finally, the conjugate is purified on a FPLC and the N-terminal end is deprotected by incubation in 20% piperidine in DMF, and is then purified again.

It must be noted that as used in this specification and the appended claims, the singular forms “a”, “an” and “the” include plural references unless the context clearly dictates otherwise. Unless defined otherwise all technical and scientific terms used herein have the same meaning as commonly understood to one of ordinary skill in the art to which this invention belongs.

### REFERENCES

Abuchowski, A. et al.; Cancer Therapy with Chemically Modified Enzymes. I. Antitumor Properties of Polyethyleneglycol-Asparaginase Conjugates, Cancer Biochem. Biophys., 7; 175-186, Gordon and Breach Science Publishers, Inc. (1984)
Braun, D., Cherdron, H., Rehahn, M., Ritter, H., Voit, B.; Polymer Synthesis: Theory and Practice, Fundamentals, Methods, Experiments, Springer New York, London, Berlin 4th ed., 2005.
Brunton, Chapter 38 In: Goodman & Gilman's The Pharmacological Basis of Therapeutics, 9th Ed., Hardman et al., eds., McGraw-Hill, New York, N.Y., pages 934-935 (1996)
Buur et al., J. Control Rel. 14, 43-51 (1990)
Carrington, L. et al.: “IL-10 antibodies to TGF-beta2 and PDGF inhibit RPE-mediated retinal concentration”, Invest. Ophthalmol. Vis. Sci., 2000, 41(5), 1210-1216.
Carrington, L M. et al.: “Differential regulation of key stages in early corneal wound healing by TGF-beta isoforms and their inhibitor”, Invest. Ophthalmol. Vis. Sci., 2006, 47(5), 1886-1894.
Chirila, T. V., Rakoczy, P. E. Garetti, K. L. Lou, S., Constable, I. J.; Review: The use of synthetic polymers for delivery of therapeutic antisense oligodeoxynucleotides. Biomaterial 23 321-342 (2001)
Chonn et al., Current Op. Biotech., 6, 698-708 (1995)
El-Hariri et al., J. Pharm. Pharmacol., 44, 651-654 (1992)
Gualtieri, F.; New Trends in Synthetic Medicinal Chemistry: Vol. 7 Methods and Principles in Medicinal Chemistry, Chapter), Chemistry of Antisense Oligonucleotides, edited by R. Mannhold, H Kuinyi, H. Timmerman, Wiley-VCH, Weinheim (Germany) New York, Toronto
Hayashi T. Tanaka H. Tanaka J. Wang R. Averbook B J, Cohen P A, Shu S., et al., Immunogenicity and therapeutic efficacy of dendritic-tumor hybrid cells generated by electrofusion, Clin Immunol. 2002 July; 104(1):14-20
Parkhurst M R, DePan C, Riley J P, Rosenberg S A, Shu S., et al., Hybrids of dendritic cells and tumor cells generated by electrofusion simultaneously present immunodominant epitopes from multiple human tumor-associated antigens in the context of MHC class I and class II molecules., J Immunol. 2003 May 15; 170(10):5317-25.
Phan V, Errington F, Cheong S C, Kottke T, Gough M, Altmann S, Brandenburger A, Emery S, Strome S, Bateman A, Bonnotte B. Melcher A, Vile R., A new genetic method to generate and isolate small, short-lived but highly potent dendritic cell-tumor cell hybrid vaccines., Nat. Med. 2003 September; 9(9):1215-9. Epub 2003 Aug. 17.
Hecht; M.; Bioorganic Chemistry: Nucleic acid, Chapter 2; The Chemical Synthesis of DNA/RNA, Oxford University Press, New York, Oxford (1996)
Hermanson, G. T.; Bioconjugate Techniques, Academic Press, New York, Boston, London (1996)
Hooftman, G. et al.,; Review Poly(ethylene glycol)s with Reactive Endgroups. U. Practical Consideration for the Preparation of Protein-PEG Conjugates, J. Bioactive Compat. Polymers, 11; 135-159, Technomic Publishing Co., Inc. (April 1996)
Jäschke, A. et al.: “Synthesis and properties of oligodeoxyribonucleotide-polyethyleneglycol conjugates”, Nuc. Acid Res., 1994, Vol. 22, No. 22, 4810-4817.
Ladd, D., Snow, R.; Reagents for the Preparation of Chromophorically Labeled Polyethylene Glycol-Protein Conjugates, Anal. Biochem., 210; 258-261, Academic Press, Inc. (1993)
Lee et al.; Critical Reviews in Therapeutic Drug Carrier Systems 8, 91-192 (1991)
Muranishi; Critical Reviews in Therapeutic Drug Carrier Systems 7, 1-33 (1990)
Nathan, A. at al. Copolymers of Lysine and Polyethyleneglycol: A New Family of Functionalized Drug Carriers, Bioconj. Chem. 4; 54-62, American Chemical Society (1993)
Nielsen, P. E. Engholm, M., Berg R. H. Buchardt, O.; Sequence-selective recognition of DNA by strand displacement with a thymine-substituted polyamide. Science 1991, 254(5037), 1497-1500 (1991)
Nielsen p. E.; Review: A DNA mimic with a peptide backbone, Bioconjug. Chem. 5(1) 3-7 (1994)
Schlingensiepen, R., Brysch, W., Schlingensiepen, K.-H.; Antisense—From Technology to Therapy, Blackwell Science, Berlin, Vienna (1997)
Takahashi et al., J. Pharm. Pharmacol., 40, 252-257 (1988)
Wojtowicz-Praga, S., Invest. New Drugs: “Reversal of tumor-induced immunosuppression by TGF-beta inhibitors”, 21(1), 21-32 (2003)
Zalipsky, S. at al., Chemistry of polyethyleneglycol conjugates with biologically active molecules, Adv. Drug Del. Reviews., 16; 157-182, Elsevier Science B. V. (1995)
Zalipsky, S. et al., Peptide Attachment to Extremities of Liposomal Surface Grafted PEG Chains: Preparation of the Long-Circulating Form of Laminin Pentapeptide, YIGSR, Bioconj. Chem., 6; 705-708, American Chemical Society (1995)
Zhao and Harris, ACS Symposium Series 680, 458-72 (1997)
Yamashita et al., J. Pharm. Pharmacol., 39, 621-626 (1987)

### Patente und Publikationen

U.S. Pat. No. 5,719,262
U.S. Pat. No. 5,714,331
U.S. Pat. No. 5,700,922
U.S. Pat. No. 5,652,356
U.S. Pat. No. 5,652,355
U.S. Pat. No. 5,623,065
U.S. Pat. No. 5,565,350
U.S. Pat. No. 5,491,133
U.S. Pat. No. 5,403,711
U.S. Pat. No. 5,366,878
U.S. Pat. No. 5,539,082
U.S. Pat. No. 5,256,775
U.S. Pat. No. 5,220,007
U.S. Pat. No. 5,149,797
U.S. Pat. No. 5,013,830
U.S. Pat. No. 3,687,808
WO 2007/109097
WO 2006/096222
WO 2005/111238
WO 2005/084712
WO 2005/059133
WO 2005/014812
WO 03/06445
WO 01/68122
WO 01/68146
WO 99/63975
WO 98/33904
WO 95/17507
WO 95/02051
WO 94/25588

1-23. (canceled)

24. A conjugate or compound comprising polyethylene glycol and an oligonucleotide, wherein at least one polyethylene glycol is linked to the 5′-end of the oligonucleotide and at least one polyethylene glycol is linked to the 3′-end of the oligonucleotide, wherein the molecular weight of the polyethylene glycol linked to the 5′- and 3′-end of the oligonucleotide is identical and is <5000 Da, or wherein the molecular weight of the polyethylene glycol linked to the 5′- and 3′-end of the oligonucleotide is different.

25. The conjugate or compound according to claim 24, wherein the polyethylene glycol linked to the 5′-end of the oligonucleotide has the 1.5- to 100-fold molecular weight of the polyethylene glycol linked to the 3′-end of the oligonucleotide, or 3′-end of the oligonucleotide the 1.5- to 100-fold molecular weight of the polyethylene glycol linked to the 5′-end of the oligonucleotide, or the polyalkylen oxide linked to the 5′-end or 3′-end of the oligonucleotide has the 1.5- to 100-fold molecular weight of the polyalkylen oxide linked a phosphate group, a sugar moiety and/or a base of the oligonucleotide, or the polyalkylen oxide linked to the phosphate group, the sugar moiety and/or any base of the oligonucleotide has the 1.5- to 100-fold molecular weight of the polyalkylen oxide linked to the 5′-end or 3′-end of the oligonucleotide.

26. The conjugate or compound according to claim 24, wherein the molecular weight of the polyethylene glycol linked to the 5′-end and the 3′-end is identical and the molecular weight of the polyethylene glycol linked to the 5′-end and the 3′-end is 200, 300, 400, 500, 600, 700, 800, 900, 1000, 1250, 1500, 1750, 2000, 2250, 2500, 2750, 3000, 3250, 3500, 3750, 4000, 4250, 4500 or 4750 Da.

27. The conjugate or compound according to claim 24, wherein the molecular weight of the polyethylene glycol linked to the 5′-end and the 3′-end is different and the molecular weight of the polyethylene glycol linked to the 5′-end is 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000 Da and the molecular weight of the polyethylene glycol linked to the 3′-end of the oligonucleotide is 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 10000, 20000, 50000 or 1000000 Da.

28. The conjugate or compound according to claim 24, wherein the oligonucleotide is an antisense oligonucleotide, an aptamer, a spiegelmer, shRNA, miRNA, RNAi and/or CpG-oligonucleotides.

29. The conjugate or compound according to claim 24, wherein the oligonucleotide hybridizes with mRNA of a molecule negatively influencing a physiological and/or biochemical effect in a cell, or hybridizes with mRNA of the receptor of the molecule.

30. The conjugate or compound according to claim 24, wherein the oligonucleotide hybridizes with mRNA of TGF-beta1, TGF-beta2, TGF-beta3, VEGF, IL-10, c-jun, c-fos, c-erbb2 (Her-2), MIA, and/or its receptor.

31. The conjugate or compound according to claim 24, wherein the oligonucleotide is SEQ ID No.: 1 (the nucleotide sequence: CGGCATGTCTATTTTGTA) and/or SEQ ID No.: 28 (the nucleotide sequence: CTGATGTGTTGAAGAACA).

32. The conjugate or compound according to claim 24 comprising more than one oligonucleotide.

33. The conjugate or compound according to claim 24 comprising at least one linker and/or spacer.

34. The conjugate or compound according to claim 24, wherein the linker is a zero length linker, a homobifunctional linker, and/or a heterobifunctional linker.

35. Method for the production of a conjugate or a compound according to claim 24, comprising the following steps
a) isolating or synthesizing the oligonucleotide,
b) protecting the 5′-end or the 3′-end of the oligonucleotide, and/or
a phosphate group, a sugar moiety, and/or a base of the oligonucleotide,
c) linking at least one polyalkylen oxide to the unprotected 3′-end or 5′-end of the oligonucleotide.

36. Method of claim 35 further comprising the steps:
d) deprotecting the protected 5′-end or 3′-end of the oligonucleotide, and/or
a phosphate group, a sugar moiety, and/or a base of the oligonucleotide,
e) linking at least one polyalkylen oxide with the deprotected 5′-end or 3′-end of the oligonucleotide, and/or the deprotected phosphate group,
sugar moiety, and/or
base of the oligonucleotide.

37. A pharmaceutical composition comprising a conjugate or compound according to claim 24 and a pharmaceutically acceptable carrier.

38. Method for the production of a pharmaceutical composition comprising adding a pharmaceutically acceptable carrier to the conjugate or compound produced according to the method of claim 35.

39. Method of controlling, preventing, or treating a disease or disorder, wherein the disease or disorder is cancer, fibrosis, or viral disease or disorder, comprising administering the conjugate or compound to a patient in need thereof.

40. The method according to claim 39, wherein the disease or disorder is a cancer selected from the group consisting of solid tumors, blood born tumors, leukemias, tumor metastasis, hemangiomas, acoustic neuromas, neurofibromas, trachomas, pyogenic granulomas, psoriasis, astrocytoma, acoustic neuroma, blastoma, Ewing's tumor, craniopharyngioma, ependymoma, medulloblastoma, glioma, hemangioblastoma, Hodgkins-lymphoma, medulloblastoma, leukaemia, mesothelioma, neuroblastoma, neurofibroma, non-Hodgkins lymphoma, pinealoma, retinoblastoma, sarcoma, seminoma, trachomas, Wilm's tumor, or is selected from the group of bile duct carcinoma, bladder carcinoma, brain tumor, breast cancer, bronchogenic carcinoma, carcinoma of the kidney, cervical cancer, choriocarcinoma, cystadenocarcinoma, embryonal carcinoma, epithelial carcinoma, esophageal cancer, cervical carcinoma, colon carcinoma, colorectal carcinoma, endometrial cancer, gallbladder cancer, gastric cancer, head cancer, liver carcinoma, lung carcinoma, medullary carcinoma, neck cancer, non-small-cell bronchogenic/lung carcinoma, ovarian cancer, pancreas carcinoma, papillary carcinoma, papillary adenocarcinoma, prostate cancer, small intestine carcinoma, prostate carcinoma, rectal cancer, renal cell carcinoma, skin cancer, small-cell bronchogenic/lung carcinoma, squamous cell carcinoma, sebaceous gland carcinoma, testicular carcinoma, and uterine cancer.

41. The method according to claim 39, wherein the disease or disorder is a viral disease or disorder selected from the group consisting of hepatitis A (HVA), hepatitis B (HVB), hepatitis C (HVC), herpes simplex virus (HSV), HIV, FIV, poliovirus, influenza virus, adenoviruses, papilloma viruses, Epstein-Barr-viruses, and small pox virus.

42. Method of using the conjugate or compound according to claim 24 comprising administering the conjugate or compound to a patient for controlling, preventing, or treating a disease or disorder, wherein the disease or disorder is cancer, fibrosis, or viral disease or disorder.

43. The method according to claim 42, wherein the disease or disorder is a cancer selected from the group consisting of solid tumors, blood born tumors, leukemias, tumor metastasis, hemangiomas, acoustic neuromas, neurofibromas, trachomas, pyogenic granulomas, psoriasis, astrocytoma, acoustic neuroma, blastoma, Ewing's tumor, craniopharyngioma, ependymoma, medulloblastoma, glioma, hemangioblastoma, Hodgkins-lymphoma, medulloblastoma, leukaemia, mesothelioma, neuroblastoma, neurofibroma, non-Hodgkins lymphoma, pinealoma, retinoblastoma, sarcoma, seminoma, trachomas, Wilm's tumor, or is selected from the group of bile duct carcinoma, bladder carcinoma, brain tumor, breast cancer, bronchogenic carcinoma, carcinoma of the kidney, cervical cancer, choriocarcinoma, cystadenocarcinoma, embryonal carcinoma, epithelial carcinoma, esophageal cancer, cervical carcinoma, colon carcinoma, colorectal carcinoma, endometrial cancer, gallbladder cancer, gastric cancer, head cancer, liver carcinoma, lung carcinoma, medullary carcinoma, neck cancer, non-small-cell bronchogenic/lung carcinoma, ovarian cancer, pancreas carcinoma, papillary carcinoma, papillary adenocarcinoma, prostate cancer, small intestine carcinoma, prostate carcinoma, rectal cancer, renal cell carcinoma, skin cancer, small-cell bronchogenic/lung carcinoma, squamous cell carcinoma, sebaceous gland carcinoma, testicular carcinoma, and uterine cancer.

44. The method according to claim 42, wherein the disease or disorder is a viral disease or disorder selected from the group consisting of hepatitis A (HVA), hepatitis B (HVB), hepatitis C (HVC), herpes simplex virus (HSV), HIV, FIV, poliovirus, influenza virus, adenoviruses, papilloma viruses, Epstein-Barr-viruses, and small pox virus.
